SEQUENCE LISTING (12 sequences)

SEQ ID NO: 1 - protein, 8 aa, Parainfluenza virus
KGLNSVOK

SEQ ID NO: 2 - protein, 8 aa, Measles morbillivirus
LSLTVELK

SEQ ID NO: 3 - protein, 8 aa, Nipah henipavirus
LSDGENPK

SEQ ID NO: 4 - protein, 8 aa, Hendra henipavirus
LSLGGDII

SEQ ID NO: 5 - protein, 8 aa, Parainfluenza virus
LRQDLQTN

SEQ ID NO: 6 - protein, 8 aa, Newcastle disease virus
KVSTSLGE

SEQ ID NO: 7 - protein, 9 aa, Parainfluenza virus
LFLEAAGLQ

SEQ ID NO: 8 - protein, 9 aa, Measles morbillivirus
NELIPSMNQ

SEQ ID NO: 9 - protein, 8 aa, Nipah henipavirus
LVPTIDKI

SEQ ID NO: 10 - protein, 9 aa, Hendra henipavirus
TNLVPSIDQ

SEQ ID NO: 11 - protein, 7 aa, Parainfluenza virus
QDHINSV

SEQ ID NO: 12 - protein, 5 aa, Newcastle disease virus
ISNIE

(19) United States
(12) Patent Application Publication
(10) Pub. No.: US 2025/0375514 A1
(43) Pub. Date: Dec. 11, 2025

(54) METHODS OF INHIBITING PARAMYXOVIRIDAE FUSION TO A TARGET CELL

(71) Applicant: THE TRUSTEES OF COLUMBIA UNIVERSITY IN THE CITY OF NEW YORK, New York, NY (US)

(72) Inventors: Anne Moscona, New York, NY (US); Tara Marcink, New York, NY (US); Matteo Porotto, New York, NY (US)

(21) Appl. No.: 19/289,459

(22) Filed: Aug. 4, 2025

Related U.S. Application Data
(63) Continuation of application No. PCT/US2024/014522, filed on Feb. 5, 2024.
(60) Provisional application No. 63/483,170, filed on Feb. 3, 2023.

(51) Int. Cl. A61K 39/155; A61K 39/00; A61P 31/14; C07K 14/005; C07K 16/10; G01N 33/569

(52) U.S. Cl. CPC A61K 39/155; A61P 31/14; C07K 14/005; C07K 16/1027; G01N 33/56983; A61K 2039/575; C07K 2319/00; C12N 2760/18622; C12N 2760/18634; G01N 2333/115; G01N 2500/02

(57) ABSTRACT

A novel anti-Paramyxoviridae viral therapeutic strategy is described herein. The strategy targets the discovery that the receptor binding protein of the virus forms a complex with the fusion protein that maintains the fusion protein in its pre-fusion configuration. Accordingly, methods and uses of the fusion complex or fragments thereof related to drug screening, antibody generation, or inhibition of membrane fusion of a Paramyxoviridae virus to a target cell are disclosed.

### CROSS-REFERENCE TO RELATED APPLICATION

This application claims priority to and the benefit of International Application No. PCT/US2024/0014522 filed Feb. 5, 2024, and U.S. Provisional Application No. 63/483,170 filed Feb. 3, 2023, the contents of each of which are hereby incorporated by reference.

### STATEMENT REGARDING FEDERALLY SPONSORED RESEARCH OR DEVELOPMENT

This invention was made with government support under All 14736, AI160961, AI121349, AI160953, and AI152275 awarded by the National Institutes of Health. The government has certain rights in the invention.

### INCORPORATION-BY-REFERENCE OF MATERIAL ELECTRONICALLY FILED

Incorporated by reference in its entirety herein is a computer-readable nucleotide/amino acid sequence listing submitted concurrently herewith and identified as follows: One 11,290 byte XML file named “44010147SeqList_Updated” created on Aug. 27, 2025.

### TECHNICAL FIELD

This disclosure relates to the discovery of a novel strategy for inhibiting fusion of Paramyxoviridae viruses to a target cell by maintaining the stability of the pre-fusion configuration of the fusion protein.

### BACKGROUND

The virus family of Paramyxoviridae includes a broad swath of human pathogens, which mainly come from three genera: Paramyxovirus, which includes but is not limited to, the parainfluenza viruses (causes infections of upper and lower respiratory illnesses) and mumps virus (causes infections of the salivary glands); Pneumovirus, which includes but is not limited to respiratory syncytial virus (causes infections of the lungs and respiratory tract); and Morbillivirus, which includes but is not limited to the measles virus (causes infections of the respiratory tract that spreads systemically). All of the viruses in this family are enveloped viruses.

Enveloped viruses have evolved surface glycoprotein complexes that mediate fusion of their envelopes with their target cell and deliver the viral genome into the target cell cytoplasm. For the family Paramyxoviridae, this complex consists of two membrane proteins that cooperate to mediate binding and cell entry. It has become clear that, for all these viruses—whether the receptor binding proteins bind sialic acid moieties, like parainfluenza virus, or proteinaceous receptors, like measles or Nipah viruses—upon receptor engagement, the receptor binding protein triggers the fusion protein to activate fusion and viral entry, which is necessary initiating disease-causing cycles of viral replication. Accordingly, targeting the membrane fusion process has been a hopeful strategy for developing therapeutics against as well as vaccinations against enveloped viruses. In spite researchers having this clear goal, development of treatment and infection-prevention products against most enveloped viruses that affect human quality of life are still needed. Part of the slow progress is due to the architecture of glycoproteins on the viral surface of enveloped viruses being largely unknown. While the viral glycoproteins have been characterized for influenza virus and HIV, information about paramyxoviruses is limited.

### SUMMARY OF THE DISCLOSURE

A method of screening for a therapeutic agent that inhibits membrane fusion by a Paramyxoviridae virus is disclosed. In one aspect, method comprises administering a small molecule or peptide to a receptor binding protein of the Paramyxoviridae virus or a fragment thereof, wherein the fragment of the receptor binding protein comprises globular head domain or a fragment thereof. The globular head domain caps the fusion (F) protein of the Paramyxoviridae virus at its apex. The binding of the small molecule or peptide to the receptor binding protein of the Paramyxoviridae virus or a fragment thereof indicates the small molecule or peptide is likely a therapeutic agent that inhibits membrane fusion by the Paramyxoviridae virus.

In some embodiments, the globular head domain of the receptor binding protein caps the F protein at its apex in its pre-fusion configuration. In certain implementations, the fragment of the receptor binding protein comprises a loop and/or a beta sheet. In particular embodiments, the fragment of the receptor binding protein comprises loop and the loop comprises an amino acid sequence set forth in: KGLNSVOK (SEQ ID NO. 1), LSLTVELK (SEQ ID NO. 2), LSDGENPK (SEQ ID NO. 3), LSLGGDII (SEQ ID NO. 4), LRQDLQTN (SEQ ID NO. 5), or KVSTSLGE (SEQ ID NO. 6).

In another aspects, the method of screening for a therapeutic agent that inhibits membrane fusion by a Paramyxoviridae virus comprises administering a small molecule or peptide to a fragment of a F protein of the Paramyxoviridae virus comprising the apex of the F protein. Binding of the small molecule or peptide to the apex of the F protein indicates the small molecule or peptide is likely a therapeutic agent that inhibits membrane fusion by the Paramyxoviridae virus. In certain implementations, the apex of the F protein comprises an amino acid sequence selected from the group consisting of: LFLEAAGLQ (SEQ ID NO. 7), NELIPSMNQ (SEQ ID NO. 8), LVPTIDKI (SEQ ID NO. 9), TNLVPSIDQ (SEQ ID NO. 10), QDHINSV (SEQ ID NO. 11), and ISNIE (SEQ ID NO. 12). In such embodiments, the Paramyxoviridae virus is selected from the group consisting of: measles virus, Nipah virus, Hendra virus, Newcastle disease virus, and parainfluenza virus. In some aspects, the parainfluenza virus is human parainfluenza virus 3 or parainfluenza virus 5.

In yet another aspects, the method of screening for a therapeutic agent that inhibits membrane fusion by a Paramyxoviridae virus comprises administering a small molecule or peptide to a receptor binding protein of the Paramyxoviridae virus or a fragment thereof, wherein the fragment of the Paramyxoviridae virus comprises globular head domain of the receptor binding protein or a fragment thereof, wherein the globular head domain caps the F protein of the Paramyxoviridae virus at its apex; and administering the small molecule or peptide to a fragment of the F protein comprising the apex of the F protein. Binding of the small molecule or peptide to the receptor binding protein of the Paramyxoviridae virus or a fragment thereof or to the apex of the F protein of the Paramyxoviridae virus or a fragment thereof indicates the small molecule or peptide is likely a therapeutic agent that inhibits membrane fusion by the Paramyxoviridae virus.

In some implementations, the globular head domain of the receptor binding protein caps the F protein at its apex in its pre-fusion configuration. In some aspects, the fragment of the receptor binding protein comprises a loop and/or a beta sheet. In particular embodiments, the fragment of the receptor binding protein comprises loop and the loop region comprises an amino acid sequence set forth in: KGLNSVOK (SEQ ID NO. 1), LSLTVELK (SEQ ID NO. 2), LSDGENPK (SEQ ID NO. 3), LSLGGDII (SEQ ID NO. 4), LRQDLQTN (SEQ ID NO. 5), or KVSTSLGE (SEQ ID NO. 6). In some aspects, the apex of the F protein comprises an amino acid sequence selected from the group consisting of: LFLEAAGLQ (SEQ ID NO. 7), NELIPSMNQ (SEQ ID NO. 8), LVPTIDKI (SEQ ID NO. 9), TNLVPSIDQ (SEQ ID NO. 10), QDHINSV (SEQ ID NO. 11), and ISNIE (SEQ ID NO. 12). In such embodiments, the Paramyxoviridae virus is selected from the group consisting of: measles virus, Nipah virus, Hendra virus, Newcastle disease virus, and parainfluenza virus. In some aspects, the parainfluenza virus is human parainfluenza virus 3 or parainfluenza virus 5.

A method of generating a neutralizing antibody against a Paramyxoviridae virus is also described herein. The method comprises immunizing an animal with polypeptide comprising an amino acid sequence encoding at least a portion of a globular head domain of a receptor binding protein of the Paramyxoviridae virus, wherein the globular head domain caps the F protein of the Paramyxoviridae virus at its apex. In some aspects, the globular head domain of the receptor binding protein caps the F protein in its pre-fusion configuration. In certain implementations, the at least one portion of a globular head domain of the receptor binding protein comprises a loop and/or a beta sheet. In particular implementations, the polypeptide comprises an amino acid sequence set forth in SEQ ID NOs 1-6. In such implementations, the Paramyxoviridae virus is selected from the group consisting of: measles virus, Nipah virus, Hendra virus, Newcastle disease virus, and parainfluenza virus (for example, human parainfluenza virus 3 or parainfluenza virus 5).

In another implementation of the method of generating a neutralizing antibody against a Paramyxoviridae virus, the method comprising immunizing an animal with polypeptide comprising an amino acid sequence encoding a fragment of F protein of the Paramyxoviridae virus, wherein the fragment of F protein comprises its apex. In some aspects, the polypeptide comprises an amino acid sequence set forth in SEQ ID NOs. 7-12. In such implementations, the Paramyxoviridae virus is selected from the group consisting of: measles virus, Nipah virus, Hendra virus, Newcastle disease virus, and parainfluenza virus (for example, human parainfluenza virus 3 or parainfluenza virus 5).

A method of inhibiting membrane fusion of a Paramyxoviridae virus to a target cell is also described. In one aspect, the method comprises administering to the target cell a therapeutic peptide or protein comprising a globular head domain of a receptor binding protein of the Paramyxoviridae virus or a fragment thereof, wherein the globular head domain caps the F protein of the Paramyxoviridae virus at its apex. In another aspects, the method comprises administering to the target cell an antibody, wherein the antibody comprises an antigen binding site comprising an amino acid sequence encoding a globular head domain of a receptor binding protein of the Paramyxoviridae virus or a fragment thereof, wherein the globular head domain caps the F protein of the Paramyxoviridae virus at its apex. In some aspects, the fragment of the receptor binding protein comprises a loop and/or a beta sheet.

A method of stabilizing fusion (F) protein of a Paramyxoviridae virus in a pre-fusion configuration is further described. The method comprising administering to the F protein a therapeutic peptide or protein comprising an amino acid sequence of 5-30 residues in length encoding a globular head domain of a receptor binding protein of the Paramyxoviridae virus or a fragment thereof. The globular head domain caps the F protein of the Paramyxoviridae virus at its apex. The fragment of the receptor binding protein comprises a loop and/or a beta sheet.

Uses of a fragment of a Paramyxoviridae virus receptor binding protein for the manufacture of medicament or for inhibiting membrane fusion of the Paramyxoviridae virus to a target cell are further described. The fragment of Paramyxoviridae virus receptor binding protein comprises a globular head. The fragment of the Paramyxoviridae virus receptor binding protein comprises at least one portion of the globular head. The at least one portion of the globular head binds to F protein of the Paramyxoviridae virus. In some aspects, the fragment of a Paramyxoviridae virus receptor binding protein comprises at least one portion of the globular head domain that caps the apex of the F protein. For example, the apex of the F protein comprises an amino acid sequence selected from SEQ ID NOs. 7-12. In some aspect, the fragment of a Paramyxoviridae virus receptor binding protein comprises a loop and/or a beta sheet. For example, the fragment of a Paramyxoviridae virus receptor binding protein comprises a loop, the fragment comprises an amino acid sequence set forth in SEQ ID NOs. 1-6.

The foregoing and other aspects, features, and advantages will be apparent from the DESCRIPTION and DRAWINGS, and from the CLAIMS if any are included.

### BRIEF DESCRIPTION OF THE DRAWINGS

Implementations will hereinafter be described in conjunction with the appended and/or included DRAWINGS, where like designations denote like elements. The patent or application file contains at least one drawing executed in color. Copies of this patent or patent application publication with color drawing(s) will be provided by the Office upon request and payment of the necessary fee.

FIGS. 1A-J show schematics of the subnanometer resolution of the HN/F viral fusion complex on the virus surface. FIG. 1A depicts a representative central slice from a tomogram of an HPIV3 viral particle. FIG. 1B depicts X and Y projections from final subtomogram complex reconstruction using a tight mask around HN and F. FIG. 1C depicts a Fourier shell correlation (FSC) plots of the HN and F complex with and without a mask and the individual HN- and F-focused refinements. Side views and top view of the final HN (green) and F (pink) reconstruction without (FIGS. 1D and 1E) and with (FIGS. 1F and 1G) prefusion F [Protein Data Bank (PDB) ID: 6MJZ] and HN (PDB ID: 4MZA) models fit into the density. Side view of the prefusion F apex region with clear α-helical densities is shown in the inset. FIG. 1G shows HN and F each separately in top-down view with the models fit into the density. FIG. 1H provides a side view of the HN/F complex reconstruction filtered to 20 Å and with prefusion F (PDB ID: 6MJZ) and HN (PDB ID: 4MZA) models fit into the density. The density threshold level was decreased to reveal the viral membrane. FIG. 1I depicts a full-length HN (green model) and F (pink model) derived from a combination of the soluble F structure (PDB ID: 6MJZ) and the AlphaFold model of the F trimer stalk region were fit into our final subvolumes. FIG. 1J show a subtomogram-averaged volume of the glycoprotein organization on the viral surface (one of the five classes from a particle classification is shown, revealing separated dimers). Scale bars: FIG. 1A—50 nm, FIG. 1D—5 nm, and FIG. 1J—10 nm.

FIGS. 2A-2H show schematics of the interactions between HN protomer heads observed in cryo-ET reconstructions compared with the crystal structure of soluble HN dimer. FIG. 2A shows a full-length cryo-ET AlphaFold model of HN+F. Dashed box: HN protomers individually fit into cryo-ET density. FIG. 2B shows a crystal structure model of dimeric HN (PDB ID: 4MZA) fit into cryo-ET density. Axis of rotation around HN (Z′; purple dashed lines) plotted in relation to viral membrane. When compared to the z axis of viral membrane (gray cube), axis of rotation around HN is tilted 5° for the cryo-ET model (FIG. 2A) and 25° for the crystal structure model (FIG. 2B) (purple cubes). FIG. 2C shows zoomed-in side, top views of individual HN protomer heads from crystal structure (green) fit into cryo-ET density. FIG. 2D shows zoomed-in side, top views of HN dimer heads from crystal structure (gray) placed into cryo-ET density by fitting only proximal HN protomer head. FIG. 2E shows a cryo-ET model has 156° between HN protomer heads with respect to the Z′ axis (purple bar) and 51 Å between the HN protomer centers (red spheres). FIG. 2F show Crystal structure model has 178° between the two HN protomers' heads with respect to the Z′ axis (purple bar) and 43 Å between the centers. FIGS. 2G and 2H show peeling opens the HN dimer for each model so that the interfaces face out. Surfaces that contact the opposing protomer in the cryo-ET model are red (FIG. 2G). Buried surface area in the cryo-ET structure (red) is less than the buried surface area in the crystal structure (FIG. 2H). Scale bars: (FIGS. 2A-2D)—5 nm.

FIGS. 3A-3J show schematics of the loop motif of the HN protomeric head interacting with the apex of F. FIG. 3A shows a full-length cryo-ET AlphaFold-derived model of HN+F fit into the cryo-ET density. The dashed box indicates the HN/F interaction region under discussion. FIG. 3B shows a zoomed-in view of the final reconstruction focused on the HN/F interaction. FIG. 3C depicts models of HN (green) and F (pink) fit into the density of FIG. 3A with the HN loop interacting with the apex of F. FIG. 3D shows electrostatic potential of the HN/F interaction region under discussion. FIG. 3E shows Individual residues of HN and F (spheres) that are in close interaction with each other at the F apex. FIG. 3F show density corresponding to an N-linked glycosylation site that is involved with the HN/F interaction region. The model has one hydrogen bond between the apex of F and the sugar molecule on HN. FIG. 3G shows schematic side views of the thumb-finger motif that appears to position the HN protomer above F. FIGS. 3H-3J) show overlay of measles H (yellow) and Nipah G (red) on the HPIV3 HN protomer (green) with comparison showing the similarities in receptor binding site 1 (FIG. 3H) along with the thumb finger motif (FIGS. 3I and 3J);

FIG. 4A-4M show schematics of the anti-F-neutralizing antibody and HN binding at the apex of prefusion F. FIG. 4A shows cryo-EM structure of soluble prefusion F (pink) bound to anti-F-neutralizing PIA174 Fab (blue) (PDB ID: 6MJZ). FIGS. 4B and 4C show overlay of PIA174 Fab surface (blue) with (FIG. 4B) and without (FIG. 4C) the surface from the cryo-ET model with Fab (blue), HN (green), and F (pink). FIGS. 4D and 4E show disruption of viral surface by PIA174 Fab showing regions of viral surface with no density (orange arrows) or F only without HN (pink arrows). FIGS. 4F and 4G show subtomogram average without (FIG. 4F) and with (FIG. 4G) the fit of the PIA174 Fab cryo-EM structure. FIG. 4H shows X and Y projection slices from subtomogram reconstruction of selected particles on the viral surface containing PIA174 bound to prefusion F. FIG. 4I shows plots of FSCs from the final subvolume of the PIA174 Fab bound to F complex with masked and unmasked resolution at the 0.143-Å cutoff value. FIG. 4J depict a schematic (FIG. 4J) of assay for PIA174 Fab stabilization of prefusion F (heat temperature-mediated activation of F at 55° C.) in the absence of HN. FIG. 4K shows percent cells with prefusion F after varying incubation durations at 55° C. with prefusion-specific PIA174 Fab, normalized to F alone and incubated with Fab at 4° C. ***P≤0.001 by two-way analysis of variance (ANOVA) and Sidak's post hoc test. FIG. 4L shows a schematic of competition between HN and PIA174.

FIG. 4M depicts the proportion of PIA174 Fab binding to cells expressing F alone (pink bar) or F+HN (green bar) in presence versus absence of DTSSP. Data are means±SE from three separate experiments. **P≤0.01 by unpaired two-tailed t test. Scale bars: FIGS. 4D and 4E—50 nm and FIG. 4F—10 nm.

FIGS. 5A-5L show schematics and graphs depicting the alterations at key HN/F interface probed with neutralizing anti-F antibody. FIG. 5A show the virus that arose under selective pressure of PIA174 Fab is resistant to entry inhibition by PIA174 Fab. Inhibition was quantitated by plaque counting normalized to no treatment. FIG. 5B shows F-A194T is not recognized by PIA174 Ab on cells expressing F-parental or F-A194T and treated with PIA174 Ab. FIGS. 5C-5E are a top-down view of the soluble cryo-EM prefusion F structure (PDB ID: 6MJZ) (FIG. 5C) F (pink surface), F with A194T mutation (FIG. 5D) (orange surface), and F-A194T mutation and PIA174 Fab loop (FIG. 5E) (blue spheres). FIGS. 5F and 5G are schematics of glycoprotein combinations used in the next set of experiments: F+HN or F+influenza HA, which tethers via sialic acid receptor engagement but does not complex with, or activate, F, with or without PIA174 Ab. FIG. 5H show inhibitory activity of PIA174 Ab in cell-cell fusion assay based on a complementation of β-galactosidase (β-Gal) where cell fusion leads to alpha-omega complementation. Receptor-bearing cells coexpressing HN+F (green), HA+F (red), HN+F-A194T (orange), and HA+F-A194T (purple) were treated with PIA174 Ab (x axis). y axis, % inhibition of fusion by of PIA174 Ab. FIGS. 5I and 5J show activation of F by heat assessed at a range of temperatures without (FIG. 5I) or with (FIG. 5J) F-A194T mutation. Readout for F activation is fusion, binding, or release of red blood cells (RBCs) interacted with the F-expressing cells. FIGS. 5K and 5L show activation of F by HN was assessed at a range of temperatures without (FIG. 5K) or with (FIG. 5L) F-A194T. Data are means±SE from at least three separate experiments for FIGS. 5A, 5B, and 5H-5L.

FIGS. 6A-C show tomograms and schematics showing HN and F organization on the surface. FIGS. 6A and 6B show representative tomogram without (FIG. 6A) and with (FIG. 6B) individual particle positions from the final HN and F complex subtomogram averaging data placed back onto the tomogram. FIG. 6C show subtomogram averaging of the glycoprotein organization on the viral surface. The cryo-ET HN dimer model was fit into these lower density maps in the central HN/F complex. These subaverages were derived from the final HN/F complex, where the mask was relaxed to encompass just the HN layer. Scale Bar (FIG. 6C): 10 nm.

FIGS. 7A and 7B show flow diagram for the subtomogram averaging workflow. FIG. 7A show workflow for the subtomogram averaging of the HN and F complex. FIG. 7B show workflow for the subtomogram averaging of the pre-fusion F Fab antibody bound to F.

FIGS. 8A-8I are schematics and graphs for the resolution from all final subvolumes of the HN and F complex. Final filtered subvolumes of the HN and F complex (FIG. 8A), focused F refinement (FIG. 8D), focused HN refinement (FIG. 8E). Fourier shell correlation plots of each final round's masked and unmasked subvolumes with resolution at 0.143 Å cut-off value (FIGS. 8B, 8D, and 8E). Local resolution estimations for each final subvolumes as determined by ResMap (FIG. 8C). The combined cryo-ET density map filtered at higher threshold with the individual HN crystal structure (PDBID:4MZA) and the F cryo-EM structure (PDBID:6MJZ) fit into the density map (FIGS. 8F-8I). Views around the crystal structure HN dimer showing the fit of the HN dimer into the density map with a model correlation of 0.82 (FIG. 8G). Views around the cryo-ET individual HN dimer model showing the fit of the individual HN protomers into the density map with a model-to-map cross-correlation of 0.86 (FIG. 8H). Z slices through the density of pre-fusion F at 3 different regions revealing the HN protomer fit (FIG. 8I).

FIGS. 9A-9D are schematics for the comparison of the HPIV3 and NDV HN dimer orientations. The axes of rotation and distances and distances between the centers of mass (red spheres) for the HPIV3 HN dimer cryo-ET model (FIG. 9A) in the loose dimer conformation (FIG. 9C) and crystal model (FIG. 9B) in the tight dimer conformation (PDBID:4MZA) (FIG. 9D).

FIGS. 10A and 10B compares HN with other paramyxoviruses receptor binding proteins. FIG. 10A depicts sequence alignment of the HN loop region and the F apex region with analogous loop and F apex regions of other paramyxoviruses. FIG. 10B is an overlay of the final subvolume with the HN loop region (green) and measles H (yellow), NiV G (red), Hendra G (pink), PIV5 HN (blue), and NDV (purple).

FIGS. 11A-11C show pre-fusion Fab antibody bound to F subtomogram averaging resolution. FIGS. 11A and 11B show the position of individual particles from the final subtomogram average of the pre-fusion F Fab antibody bound to F placed back into a representative tomogram. FIG. 11C show fitting of a full Fab antibody fragment (PDBID:6HJQ) on top of pre-fusion F. The full Fab was fit onto the variable portion of the PIA174 model.

FIG. 12 shows F pre-fusion conformation stability in the presence of PIA174. Anti-F neutralizing antibody prevents thermally induced F refolding into its post-fusion conformation. Fluorescence intensity of post-fusion F conformation specific antibody (PA3/F4) staining following pretreatment with or without pre-fusion-specific PIA174 antibody and a 30-minute exposure to 55° C. Fluorescence was normalized to F without PIA174 antibody treatment after 30 minutes at 55° C. Data are means±standard errors (SE) from five separate experiments.

*P≤0.05 determined by unpaired two-tailed t-test.

FIG. 13 shows maximum allele frequency plots for nonsynonymous mutational changes across the whole genome during persistent HPIV3 infection. Maximum allele frequency noted for nonsynonymous mutational changes across whole genome of the virus that escaped during viral evolution under the selective pressure of the PIA174 anti-F Ab.

FIG. 14 shows cryo-tomography of the HPIV3 virion. FIG. 14A shows the central slice of a cryo-ET tomogram of the intact HPIV3 virion. FIG. 14B shows the subtomogram averaged density of the HN/F complex on the surface of a virus.

FIGS. 15A-15E show that anti-F neutralizing antibody antigen-binding fragment (Fab) binds to a site overlapping the site of HN-F interaction in the HN-F complex, stabilizes F, & inhibits fusion. FIGS. 15A and 15B compare the cryo-ET HN (green) and F (pink) complex with the cryo-EM anti-F neutralizing Fab (blue) and F complex. Zoomed in views of the interaction sites. FIG. 15C shows fusion activity of HN/F-coexpressing cells with 293T cells in the presence of serial dilutions of PIA174 Fab. FIG. 15D shows binding of pre-fusion anti-F Fab to F-alone or HN-F expressing cells. FIG. 15E shows the percent of cells with F in prefusion state after varying times at 55° C. with (blue) or without (pink) PIA174 Fab.

FIGS. 16A and 16B shows a cryo-ET visualization of the HN/F complex disrupted by the PIA174 Fab. FIG. 16A shows regions of the viral surface with no densities (orange arrows) or F without HN (pink arrows). Scale Bar: 50 nm. FIG. 16B shows subtomogram average of the F (pink) and anti-F neutralizing Fab PIA174 (blue) complex on the viral surface.

FIGS. 17A and 17B shows altered activation of F-A194T mutant. FIG. 17A shows cells coexpressing wt HN+either wt F or F-A194T were bound to receptor-bearing RBCs at 4° C. and then transferred to a range of temperatures. Y axis=RBCs that were released, bound, or fused (indicating F triggering) after 60 min. at each temp. In the presence of HN, F-A194T is harder to activate than wt F/triggers at a higher temp.) FIG. 17B show cells coexpressing influenza HA (binds SA receptor but does not interact with F)+either wt F or F-A194T were bound to receptor-bearing RBCs at 4° C. The assay proceeded as for (17A). In the absence of HN, the F-A194T is easier to activate than wt F/triggers at lower temp.

FIG. 18 shows structural comparison of paramyxovirus receptor binding proteins. Overlay of measles H (yellow) and Nipah G (red) on the HPIV3 HN monomer structure (green) in the subtomogram averaged density map. Inset shows the similarity of the HN loop around residue 386.

FIGS. 19A-19C show the position of globular head monomers with respect to each other in the cryo-ET structure of the dimer vs. the crystal structure: potential flexibility at the dimer interface. FIG. 19A shows the crystal structure of the HN globular head dimer was fit into the cryo-ET density of the HN globular head dimer from the cryo-ET HN-F complex structure. Purple bar=Z axis (perpendic. to viral membrane). FIG. 19B shows the crystal structure of individual monomers fit into the cryo-ET density. FIG. 19C first shows positions of the 3 proposed cysteine sites, shown in the crystal structure of HN globular head dimer. FIG. 19C then shows the L-hand monomer was removed and the R-hand monomer rotated to expose the dimer interface. The proposed mutations form a triangle at the dimer interface—designed to block both rotation & tilt.

FIGS. 20A-20D show the HPIV3 viral fusion process steps the pre-receptor complex, receptor engaged complex, activated fusion complex, and the post-fusion complex respectively. The top portions are schematics and the bottom portions are medium resolution subtomogram averages for each proposed step of the fusion complex. HN is shown in green and F is shown in pink.

FIGS. 21A and 21B visualize the intermediate state of F. FIG. 21A shows contrast-inverted cryo-ET images of viral particles attached to target. FIG. 21B shows subtomogram averaging of intermediate state of F (grey) at 15-20 Å resolution bridging viral (green) and host (purple) membranes. HN interacts with the intermediate state (arrow).

FIG. 22 depicts steps in entry: cryo EM before receptor engagement.

FIG. 23 shows the surface of a fresh unperturbed virion: cryo-electron to tomography of HN-F complex. Enlarged is a cryo-ET map of the viral surface away from the host cell, wherein HN and F models fit into the final complex density map. Scale is 5 nm.

FIG. 24 depicts HN globular heads, pointing in two directions.

FIG. 25 shows HN-F complex before receptor engagement, in a molecular dynamics view including stalks.

FIGS. 26A-26H show EV1 and EV2 resistance to inhibition by PIA174. Model of the full-length HN/F complex with mutations for EV1 (FIG. 26A) and EV2 (FIG. 26B) indicated by red atomic spheres. FIG. 26C show Vero cells were infected with mCherry-bearing parental, EV1, or EV2 HPIV3 and overlaid with serial dilutions of PIA174 FAb. Total fluorescence was measured two days post infection and y-axis shows the percent inhibition of total read fluorescence for each virus. FIG. 26D shows inhibition of viral entry for EV1 and EV2 was quantitated by plaque counting normalized to no treatment. FIG. 26EA shows 194T and L234F recognition by PIA174. FIG. 26F shows Cryo-EM model of prefusion F (PDBID: 6MJZ) with both PIA174 and 3x1 structures showing location of L234 (green) and A194 (red) residues in relation to 3× and PIA174 mAbs. FIGS. 26G and 26H show cells transfected with A194T, L234F, or parental Fs stabilized by Q162C-L168C, I213C-G230C, A463V, I474Y mutations were treated with 1 ug/mL PIA174 Fab or 3x1 mAb and fluorescent secondary anti-human antibody. Fluorescence was quantitated.

FIGS. 27A-27E show key PIA174 escape mutations alter the activatability of F. Percent of F in pre-fusion state at different time points at 55 C without (FIG. 27A) or with (FIG. 27B) PIA174 antibody fragment. FIG. 27C shows the rate of conformational change of uncleaved F with A194T or L234F measured by rate of lipopeptide capture of at 37° C. with Red blood cells. The HN used in this experiment bears a mutation (D216R) that makes it sialidase-deficient to maximize the HN-receptor contact and HN's fusion promotion in this experiment in order to compare the properties of the F proteins. FIGS. 27D and 27E show triggering of F by heat (left) assessed at a range of temperatures with parental, A194T, or L234F Fs. F activation is quantitated by % of red blood cells (RBCs) released, bound, or fused with F-expressing cells. Activation of F by HN (right) was assessed at a range of temperatures with parental, A194T, L234F F.

FIGS. 28A and 28B show HN escape mutations enhance fusion promotion of F. FIG. 28A shows cell-to-cell fusion measured by beta-galactosidase complementation with cells expressing HN or HA and parental F, A194T F, or L234F. Fusion values are normalized to max value in each experiment. FIG. 28B shows cell-to-cell fusion measured by beta-galactosidase.

FIGS. 29A-29I show HN/F fusion complex subtomogram averages for EV1 and EV2 variants. FIG. 29A shows side views of the field strain viral HN-F fusion complex with prefusion F [Protein Data Bank (PDB) ID: 6MJZ] and HN (PDB ID: 4MZA) models fit into the density. FIG. 29B shows side views of the EV1 HN-F reconstruction with prefusion F and HN models fit into the density. Insets show the interaction between the HN and F models for field strain and EV1 viral fusion complexes. FIG. 29C show overlay of the final subtomogram averages for field strain (blue) and EV1 (orange) fusion complexes. FIG. 29D shows 90 degrees rotated view (with respect to A) of the field strain viral fusion complex with prefusion F and HN models fit into the densities.

FIG. 29E shows side views of the EV2 HN-F reconstruction with prefusion F and HN models fit into the density. Insets show the interaction between the HN and F models for field strain and EV2 viral fusion complexes. FIG. 29F show overlay of the subtomogram averages for field strain (blue) and EV2 (green) fusion complexes. FIG. 29G shows overlay of EV2 HN/F complex (green) with L234F HN/F complex (blue). FIG. 29H shows The buried surface area (residues of interaction with HN) on F for the field strain (blue), EV1 (orange), and EV2 (green). FIG. 29I shows buried surface area (residues of interaction with HN) on F, overlaid with the model of PIA174 Fab to show overlap between Fab and HN interacting residues.

FIGS. 30A-30D show fitness cost of escape mutations. FIG. 30A shows titer measures of human Airway Epithelial (HAE) cells were infected with EV1, EV2, or Parental HPIV3 and apical washes were titered daily up to 7 days post infection. FIGS. 30B and 30C show allele frequencies (points) and titers (grey bars) of EV1 (FIG. 30B) and EV2 (FIG. 30C) at 0, 2, 4, 5, and 7 days post infection. FIG. 30D shows the location of H552Q on the field strain HN model, which remained at 100% allele frequency in all wells of EV1, and compensatory mutations in EV2 wells that increased in allele frequency during the 7 days of infection for HN.

FIGS. 31A-31D show cell surface expression of F variants. FIGS. 31A and 31C are representative western blot surface expression of F variants using anti-HPIV3 F HRC antibody in parallel with antibody recognition in FIGS. 1G and 1H. (31B, 31D) Integrated band intensity for A, C and replicates using imageJ. Band Intensity for both cleaved (top band) and uncleaved (bottom band) Fs.

FIGS. 32A and 32B show the triggering of F variants by hyperfusogenic HN variants. Triggering of F variants by low neuraminidase HN variant D216R (FIG. 32A) or high avidity HN variant T193A (FIG. 32B) to allow constant receptor engagement was assessed at a range of temperatures. Dotted line represents 50% fusion for parental F with given HN.

FIGS. 33A-33C show the subtomogram averaging workflow. Workflow for the subtomogram averaging of the EV1 (FIG. 33A), EV2 (FIG. 33B), and L234F HN and F complex (FIG. 33C). Scale bars: FIGS. 33A-33C—5 nm.

FIGS. 34A-34L show HN and F organization on the surface. Representative tomogram without (FIGS. 34A,34E, and 34I) and with (FIGS. 34B, 34F, and 34J) individual particle positions from the final EV1 (FIGS. 34A and 34B), EV2 (FIGS. 34E and 34F), and L234F (FIGS. 34I and 34J) HN and F complex subtomogram averaging dataset placed back onto the tomogram. FIGS. 34C, 34G, and 34K show Fourier shell correlation plots of each final round's masked and unmasked subvolumes for EV1 (FIG. 34C), EV2 (FIG. 34G), and L234F (FIG. 34K) HN and F complex. Resolution reported at the 0.143 Å cut-off value. FIGS. 34D, 34H, and 34L show local resolution estimations for EV1 (FIG. 34D), EV2 (FIG. 34H), and L234F (FIG. 34L) final subvolumes as determined by ResMap. Scale bars: FIGS. 34A, 34B, 34E, 34F, 34I, and 34J—50 nm; FIGS. 34D, 34H, and 34L—5 nm.

FIG. 35A-35F show variations of the HN and F interactions among viral mutants. Interaction between HN and F for field strain (blue; FIG. 35A), EV1 (pink; FIG. 35B), and EV2 (green; FIG. 35C). Rotated and expanded view of HN with buried surface area shown. FIG. 35D show side view of the L234F viral HN-F fusion complex with prefusion F [PDB ID: 6MJZ]) model fit into the density. FIG. 35E shows enlarged top-down view of the prefusion F apex with no density in between the central helices (CH1-CH3). FIG. 35F shows enlarged view of the domain adjacent to the central helix (red). Scale bar: FIG. 35D—5 nm.

### DETAILED DESCRIPTION

Detailed aspects and applications of the disclosure are described below in the following drawings and detailed description of the technology. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the disclosure. It will be understood, however, by those skilled in the relevant arts, that embodiments of the technology disclosed herein may be practiced without these specific details. It should be noted that there are many different and alternative configurations, devices, and technologies to which the disclosed technologies may be applied. The full scope of the technology disclosed herein is not limited to the examples that are described below.

The singular forms “a,” “an,” and “the” include plural referents unless the context clearly dictates otherwise. Thus, for example, reference to “a step” includes reference to one or more of such steps.

The word “exemplary,” “example,” or various forms thereof are used herein to mean serving as an example, instance, or illustration. Any aspect or design described herein as “exemplary” or as an “example” is not necessarily to be construed as preferred or advantageous over other aspects or designs. Furthermore, examples are provided solely for purposes of clarity and understanding and are not meant to limit or restrict the disclosed subject matter or relevant portions of this disclosure in any manner. It is to be appreciated that a myriad of additional or alternate examples of varying scope could have been presented but have been omitted for purposes of brevity.

When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

Throughout the description and claims of this specification, the words “comprise” and “contain” and variations of the words, for example “comprising” and “comprises”, mean “including but not limited to”, and are not intended to (and do not) exclude other components.

As used herein, the term “apex” of the fusion (F) protein refers the pocket at the top of the F protein trimer. This pocket is formed by residues found at the terminus of the central alpha-helical core and is formed by the 3 protomers in the trimer. A person having ordinary skill in the art may use conventional bioinformatics tools to identify which region(s) of the amino acid sequences of the F protein forms the apex.

As used herein, the term “caps” or “capping” refers to a receptor binding protein partially covering the apex of the fusion protein. In certain embodiments described herein, the specific interaction between HN and F of a human parainfluenza virus has a thumb finger motif of HN resting on top of the F apex. In some aspects, the interaction is the underside face of the HN protomer head lying on top of the F.

As used herein, the term “loop” refers to a segment in a protein that connect two secondary structure features. The actual structure of the loop may be irregular and depends on the length of segment. In some embodiments described herein, the term loop refers to a segment of 5-30 amino acid residues in length that do not form a secondary structure of proteins.

As used herein, the term “antibody” refers to immunoglobulins and nanobodies and recombinant forms thereof.

As required, detailed embodiments of the present disclosure are included herein. It is to be understood that the disclosed embodiments are merely exemplary of the invention that may be embodied in various forms. Therefore, specific structural and functional details disclosed herein are not to be interpreted as limits, but merely as a basis for teaching one skilled in the art to employ the present invention. The specific examples below will enable the disclosure to be better understood. However, they are given merely by way of guidance and do not imply any limitation.

The present disclosure may be understood more readily by reference to the following detailed description taken in connection with the accompanying figures and examples, which form a part of this disclosure. It is to be understood that this disclosure is not limited to the specific materials, devices, methods, applications, conditions, or parameters described and/or shown herein, and that the terminology used herein is for the purpose of describing particular embodiments by way of example only and is not intended to be limiting of the claimed inventions. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

More specifically, this disclosure, its aspects and embodiments, are not limited to the specific material types, components, methods, or other examples disclosed herein. Many additional material types, components, methods, and procedures known in the art are contemplated for use with particular implementations from this disclosure. Accordingly, for example, although particular implementations are disclosed, such implementations and implementing components may comprise any components, models, types, materials, versions, quantities, and/or the like as is known in the art for such systems and implementing components, consistent with the intended operation.

The present disclosure relates to the discovery of novel and unanticipated interactions at dimer interface of the receptor binding protein of Paramyxoviridae viruses with the fusion (F) protein that is crucial for fusion activation. The receptor binding protein and the fusion protein forms a fusion complex, and these interactions in this complex maintain the fusion protein in its pre-fusion configuration.

The series of cooperative steps that mediate enveloped virus entry have been elucidated since it was first demonstrated, using human parainfluenza virus 3 (HPIV3) as the prototype. For most Paramyxoviridae virus-induced membrane fusion, the process requires active participation of both the receptor binding protein (HN, H, or G, depending on the virus) and the fusion protein (F). While the structures of the soluble portion of individual paramyxovirus HN (or H or G) and F proteins have provided clues to the function of this HN/F fusion complex, it has also led to conflicting models for the mechanism of action of the paramyxovirus fusion complex during entry. However, the models all agree that HN, upon receptor engagement, triggers the metastable, prefusion F to undergo the series of structural transitions that result in fusion of the viral and cellular membranes.

Crystal structures of the soluble domains of receptor binding proteins are available for H protein of measles virus; G proteins of Nipah virus, Hendra virus, and respiratory syncytial virus; and NH proteins of HPIV3, human parainfluenza virus 1 (HPIV1), parainfluenza virus 5 (PIV5), and Newcastle disease virus (NDV); and for the same viruses' F proteins. These structures of soluble domains provide information on the general structure of individual glycoproteins, although do not indicate how these glycoproteins interact in a complex on the viral surface.

The type II receptor binding membrane proteins (HN for HPIV3) contain an N-terminal cytoplasmic domain, a membrane-spanning region, a stalk region, and a globular head. The four activities of HPIV3 HN—stabilization of prefusion F, receptor binding, F triggering, and receptor cleaving—are regulated precisely depending on the stage of viral entry or egress. The stalk confers specificity for the homologous F in the fusion activation process. HN's primary binding site, which has both receptor binding and receptor cleaving (neuraminidase) activities (primary sialic acid-binding site I; “site I”), is located on the globular head for HPIV3 (and other paramyxoviruses for which structural information is available).

The type I transmembrane F proteins are trimers with shorter stalks than those of HN and large globular heads. While the HPIV F protein is dissimilar in structure to other class I viral fusion proteins, it shares with others—including severe acute respiratory syndrome coronavirus disease 2 spike (S), Ebola virus glycoprotein (GP), influenza hemagglutinin (HA), and HIV Env—the strategy for membrane fusion, which entails reorientation from a small pre-fusion state to an extended intermediate state after activation. In the pre-fusion state, the F is in a metastable prefusion conformation and, for all fusion-entering viruses, must be maintained in this pre-active state by some means.

As shown in the examples, the structure of HPIV3 prefusion F was solved by cryo-electron microscopy (cryo-EM) with the aid of mutations that stabilize the prefusion state and complexed with a prefusion-specific neutralizing antibody (PIA174 Ab) that binds the apex of the prefusion F trimer at antigenic site Ø. Thus, interfering with the interface of this fusion complex resulting in prevention of F protein activation or maintenance of F protein in its pre-fusion configuration is a novel antiviral therapeutic strategy for inhibiting infections of Paramyxoviridae viruses.

For HPIV3, the fusion/entry apparatus consists of the receptor binding protein hemagglutinin-neuraminidase (HN) in complex with the fusion protein (F). F is synthesized as a metastable proprotein in its prefusion state. HN stabilizes the F protein before receptor is engaged to prevent viral inactivation. Once host cell receptors have been engaged, HN switches to new roles. Upon engagement of a cellular receptor by HN, the complex goes through a series of structural transitions, which may provide optimal targets for antibody-mediated inhibition. The HN stalk communicates with two sites in the HN head, thereby activating the trimeric F protein, inducing a conformational change in F that allows for its insertion into the host cell membrane. So the interface between HN's globular heads in the HN dimer modulates HN-F interaction and fusion. Activated F protein extends to insert into the target cell membrane and refolds to mediate virus-cell fusion and viral entry. A notable challenge to understanding these processes has been the lack of structural information about the intact receptor binding protein-fusion protein complex present in the viral membrane surface of authentic viruses.

The structures of the HN/F complex of circulating HPIV3 [clinical isolates (CIs)] in situ on the viral surface membrane before receptor engagement, presented in the examples, reveal exactly how these two molecules carry out this precise program in sequence. The structure of the complex reveals that one of the globular heads of the HN dimer caps the apex of the pre-fusion F trimer, suggesting how the pre-fusion HN-F complex is maintained in a ready but quiescent state prior to receptor engagement. An HN loop structure that appears to interact with the apex of the F trimer is highly conserved across paramyxoviruses and may be a general mechanism for maintaining the fusion/entry complex's stability and ensuring that activation of fusion occurs only at the right time and location. Interestingly, previous structural or computational analyses of the individual glycoproteins did not predict the in-situ organization of the glycoproteins in relation to each other.

Accordingly, described herein are the uses of the Paramyxoviridae virus fusion complex or a fragment of the fusion complex to screen for a therapeutic agent that inhibits membrane fusion by a Paramyxoviridae virus; to generate a neutralizing antibody against a Paramyxoviridae virus or to manufacture medicaments, for example, for inhibiting membrane fusion of a Paramyxoviridae virus to a target cell.

In one implementation, the method of screening for a therapeutic agent that inhibits membrane fusion by a Paramyxoviridae virus comprises administering a small molecule or peptide to a receptor binding protein of the Paramyxoviridae virus or a fragment thereof. The fragment of receptor binding protein comprises at least a fragment of its globular head domain, and the globular head domain caps the fusion (F) protein of the Paramyxoviridae virus at its apex. The binding of the small molecule or peptide to the receptor binding protein of the Paramyxoviridae virus or a fragment thereof indicates the small molecule or peptide is likely a therapeutic agent that inhibits membrane fusion by the Paramyxoviridae virus. In some aspects, the fragment of the receptor binding protein comprises a loop and/or a beta sheet. Where the fragment of the receptor binding protein comprises a loop, amino acid sequence encoding the loop may be selected from the group consisting of KGLNSVOK (SEQ ID NO. 1), LSLTVELK (SEQ ID NO. 2), LSDGENPK (SEQ ID NO. 3), LSLGGDII (SEQ ID NO. 4), LRQDLQTN (SEQ ID NO. 5), or KVSTSLGE (SEQ ID NO. 6). In such implementations, the Paramyxoviridae virus is selected from the group consisting of: parainfluenza virus (for example, HPIV3 or PIV5), measles virus, Nipah virus, Hendra virus, and Newcastle disease virus. The receptor binding protein in these implementations are HN for parainfluenza virus and Newcastle disease virus viruses, H for measles virus, and G for Nipah virus and Hendra virus.

In another implementation, the screening method comprises administering a small molecule or peptide to a fragment of the F protein of the Paramyxoviridae virus comprising the apex of the F protein. Binding of the small molecule or peptide to the apex of the F protein indicates the small molecule or peptide is likely a therapeutic agent that inhibits membrane fusion by the Paramyxoviridae virus. In some aspects, the apex of the F protein comprises an amino acid sequence selected from the group consisting of: LFLEAAGLQ (SEQ ID NO. 7), NELIPSMNQ (SEQ ID NO. 8), LVPTIDKI (SEQ ID NO. 9), TNLVPSIDQ (SEQ ID NO. 10), QDHINSV (SEQ ID NO. 11), and ISNIE (SEQ ID NO. 12). In such implementations, the Paramyxoviridae virus is selected from the group consisting of: parainfluenza virus (for example, HPIV3 or PIV5), measles virus, Nipah virus, Hendra virus, and Newcastle disease virus.

In certain implementations of the screening method, the small molecule or peptide is administered to a fragment of the F protein comprising the apex of the F protein as well as to the receptor binding protein of the Paramyxoviridae virus or a fragment thereof. Binding of the small molecule or peptide to the receptor binding protein of the Paramyxoviridae virus or a fragment thereof or to the apex of the F protein of the Paramyxoviridae virus or a fragment thereof indicates the small molecule or peptide is likely a therapeutic agent that inhibits membrane fusion by the Paramyxoviridae virus.

The methods of generating a neutralizing antibody against a Paramyxoviridae virus described herein use the fusion complex to generate an antibody that interferes with this interaction stabilizing the F protein in its pre-fusion configuration. In one aspect, the method comprises immunizing an animal with polypeptide comprising an amino acid sequence encoding the apex fragment of the F protein of the Paramyxoviridae virus. In certain implementations, the polypeptide comprises an amino acid sequence set forth in SEQ ID NOs. 7-12. In another aspects, the method comprises immunizing an animal with polypeptide comprising an amino acid sequence encoding at least a portion of a globular head domain of a receptor binding protein of the Paramyxoviridae virus. The globular head domain caps the F protein of the Paramyxoviridae virus at its apex. The at least one portion of a globular head domain of the receptor binding protein comprises a loop and/or a beta sheet. In some implementations, the at least one portion of a globular head domain of the receptor binding protein is encoded by an amino acid sequence set forth in SEQ ID NOs. 1-6.

For the method of inhibiting membrane fusion of a Paramyxoviridae virus to a target cell, the method comprising administering to the target cell a therapeutic peptide or protein comprising a globular head domain of a receptor binding protein of the Paramyxoviridae virus or a fragment thereof. The globular head domain caps the fusion (F) protein of the Paramyxoviridae virus at its apex. In some aspects, the fragment of the receptor binding protein comprises a loop and/or a beta sheet.

In another embodiment, the method of inhibiting membrane fusion of a Paramyxoviridae virus to a target cell comprises administering to the target cell an antibody based on the receptor binding protein. The antigen binding site of the antibody comprises an amino acid sequence encoding a globular head domain of a receptor binding protein of the Paramyxoviridae virus or a fragment thereof, wherein the globular head domain caps the fusion (F) protein of the Paramyxoviridae virus at its apex. In some aspects, the fragment of the receptor binding protein comprises a loop and/or a beta sheet.

A method of stabilizing F protein of a Paramyxoviridae virus in a pre-fusion configuration is also describe. The method comprises administering to the F protein a therapeutic peptide or protein comprising an amino acid sequence of 5-30 residues in length encoding a globular head domain of a receptor binding protein of the Paramyxoviridae virus or a fragment thereof. The globular head domain caps the F protein of the Paramyxoviridae virus at its apex. The fragment of the receptor binding protein comprises a loop and/or a beta sheet. In some aspects, the amino acid sequence of the apex region of the F protein is set forth in any one of SEQ ID NOs. 7-12. In some aspects, the fragment of the receptor binding protein comprises a loop, and the amino acid sequence of the fragment comprises an amino acid sequence set forth in any one of SEQ ID NOs. 1-6.

In some aspects, the use of a fragment of a Paramyxoviridae virus receptor binding protein for the manufacture of medicament is described, and the medicament may be used for treating or inhibiting a Paramyxoviridae virus infection. The fragment of a Paramyxoviridae virus receptor binding protein may also be used to inhibiting membrane fusion of the Paramyxoviridae virus to a target cell. The fragment of Paramyxoviridae virus receptor binding protein comprises a globular head. The fragment of the Paramyxoviridae virus receptor binding protein comprises at least one portion of the globular head and the at least one portion of the globular head binds to F protein of the Paramyxoviridae virus. In certain implementations, the at least one portion of the globular head domain caps the apex of the F protein. In some aspects, the apex of the F protein comprises an amino acid sequence selected from SEQ ID NOs. 7-12. In some embodiments, the fragment of a Paramyxoviridae virus receptor binding protein comprises a loop and/or a beta sheet. In certain implementations where the fragment of a Paramyxoviridae virus receptor binding protein comprises a loop, the fragment comprises an amino acid sequence set forth in SEQ ID NOs. 1-6. In such embodiments, the Paramyxoviridae virus is selected from the group consisting of: measles virus, Nipah virus, Hendra virus, Newcastle disease virus, and parainfluenza virus.

### EXAMPLES

The present disclosure is further illustrated by the following examples that should not be construed as limiting. The contents of all references, patents, and published patent applications cited throughout this application, as well as the Figures, are incorporated herein by reference in their entirety for all purposes.

### Example 1. Subnanometer Resolution of the HN-F Complexes on the Virus's Surface of Clinical Isolates of HPIV3

a. Sub-Nanometer Resolution of HPIV3 Fusion Entry Complex on the Surface of Clinical Isolate Virions

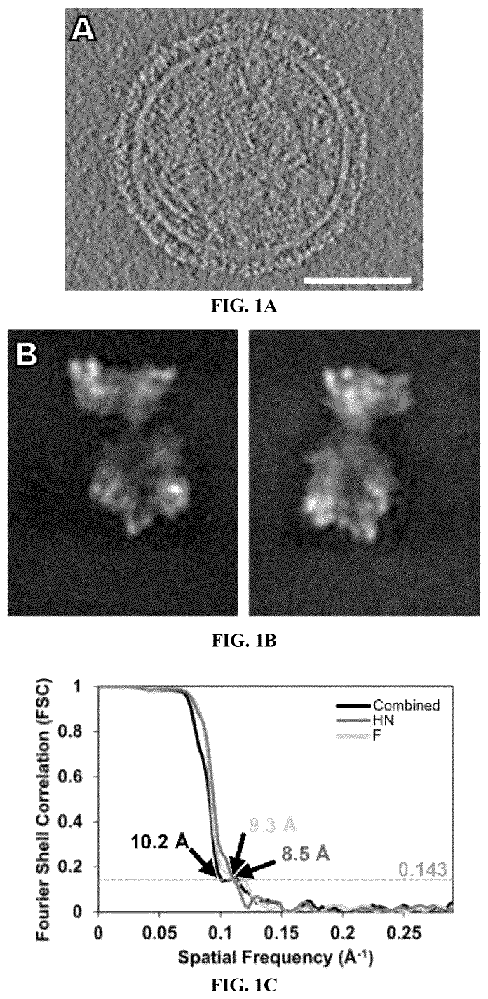

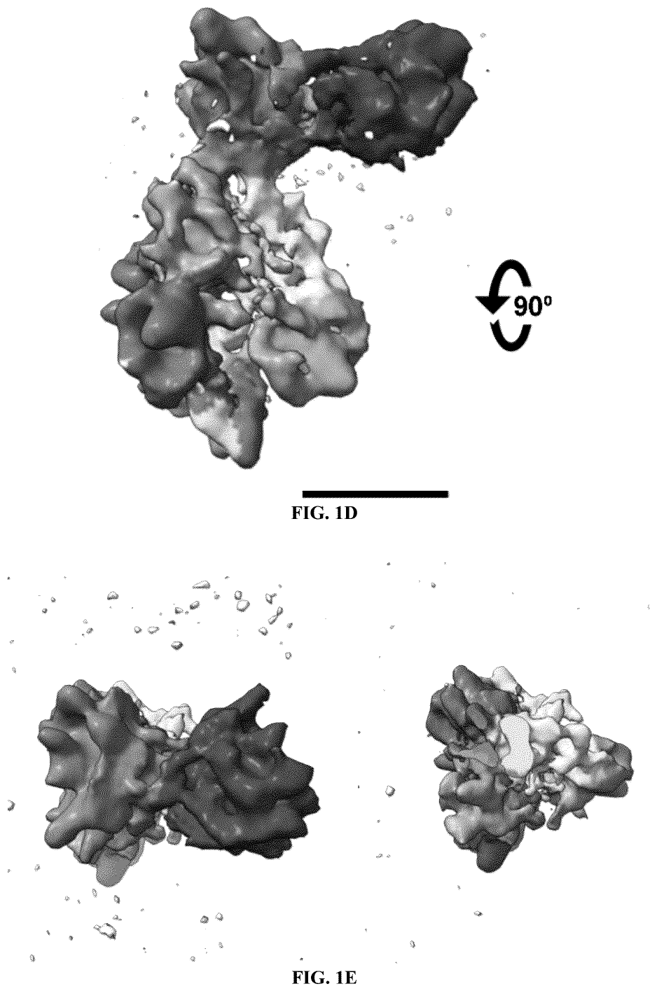

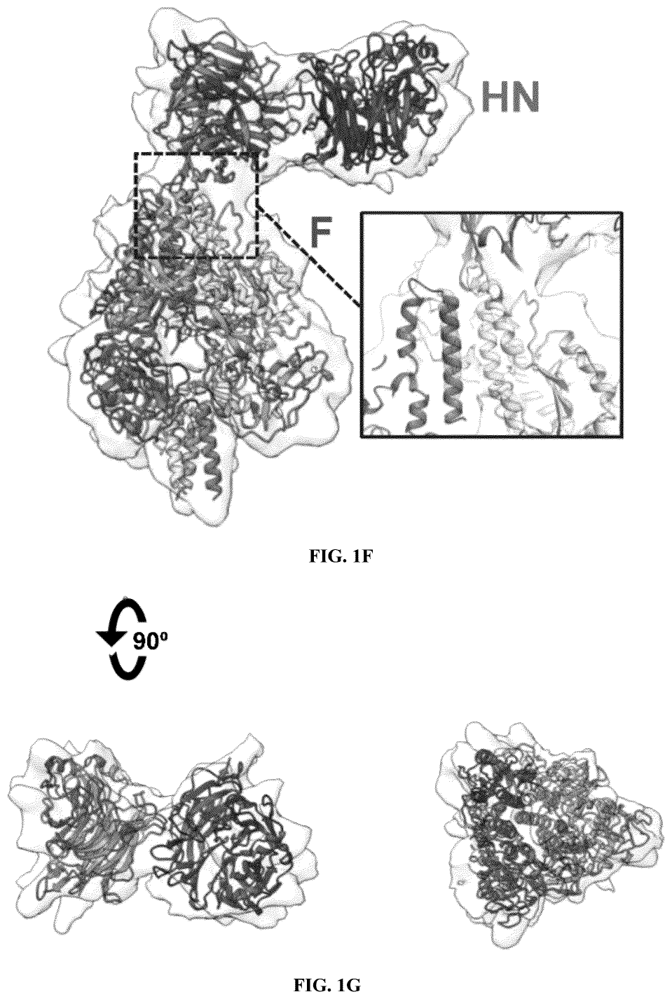

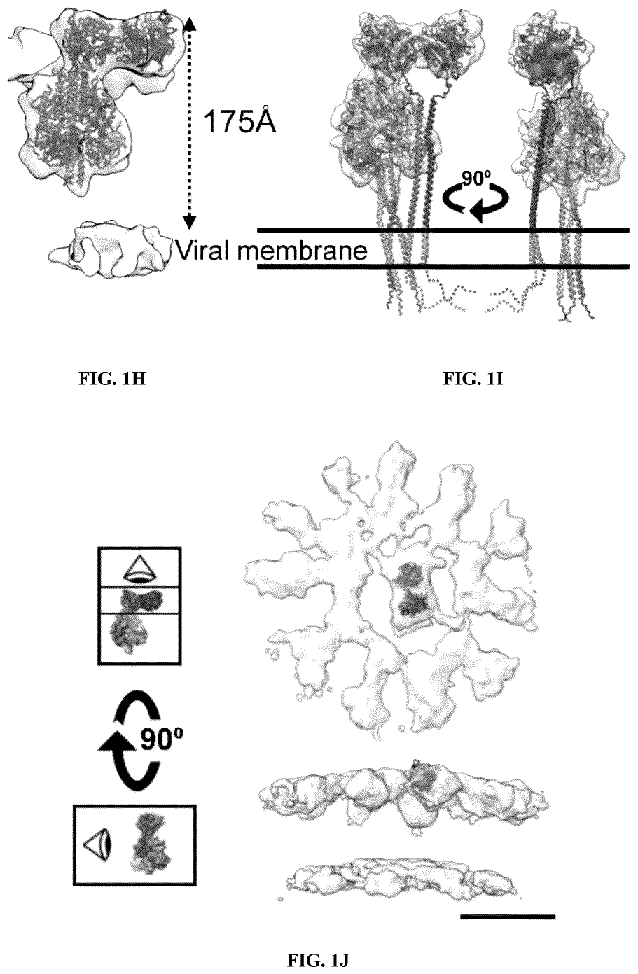

Circulating HPIV3 viruses that cause human disease bear HN/F fusion complexes that differ significantly from the complexes from laboratory-adapted strains previously used to study function and structure. The HPIV3 CIs have HN/F pairs that are poorly suited to infect cultured cells, and culture-adaptive mutations occur that alter the behavior of these complexes. In particular, the dimer interface between the HN globular heads is key in modulating fusion activation and is prone to evolve under the selective pressure of distinct infection environments. To correlate structure with function of the authentic entry apparatus, CI viruses captured directly from human airway epithelial tissue culture, or engineered viruses based on the CI genome, were used for cryo-ET structural study of authentic HN/F complexes. The viruses captured directly on grids without high-speed centrifugation or other disruptive steps bear intact lipid bilayers and complexes composed of prefusion F adjacent to and below HN molecules, with the globular heads of the HN protomers positioned above F (FIG. 1A).

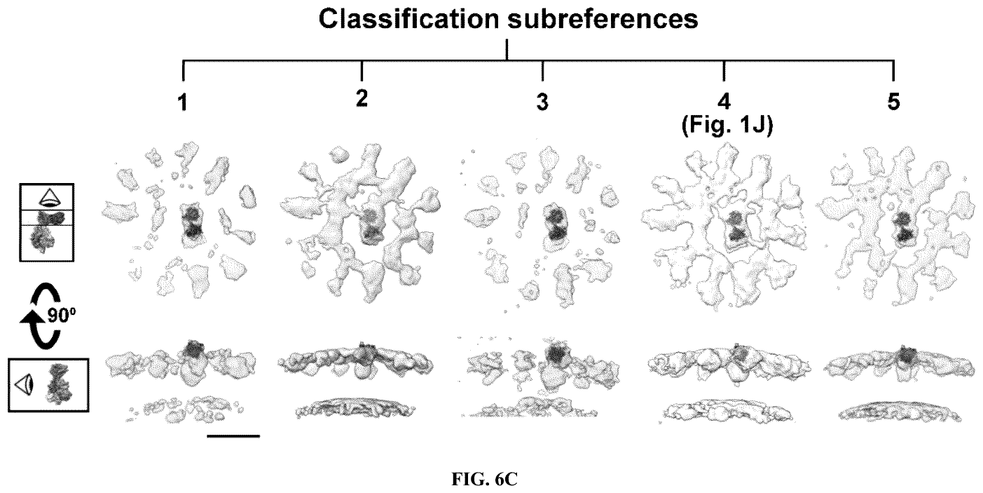

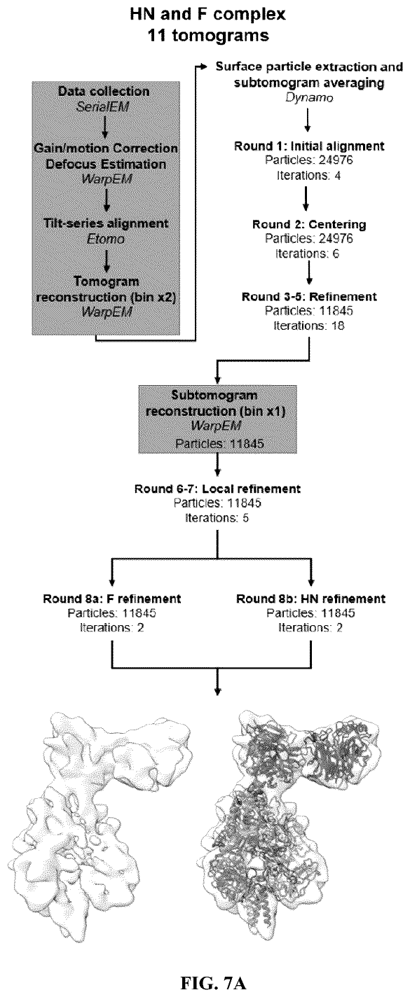

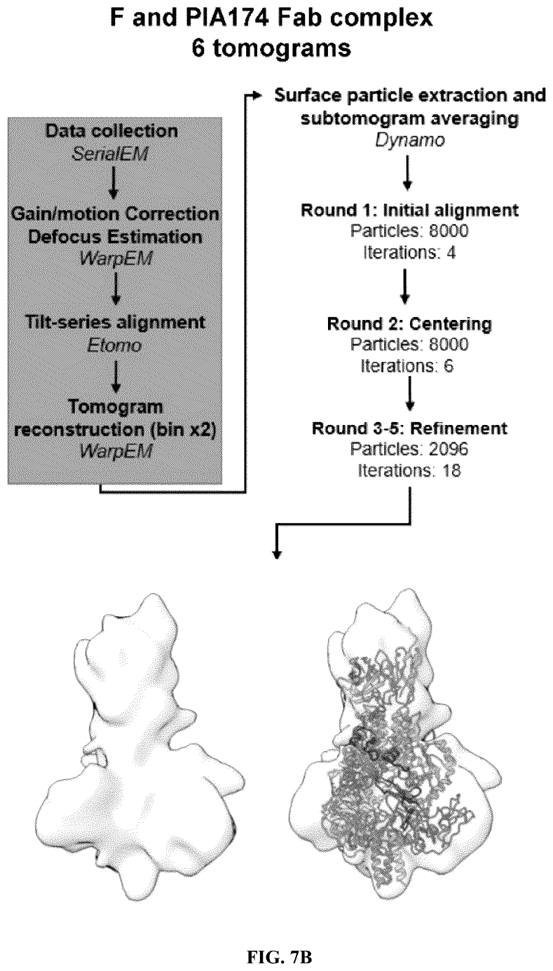

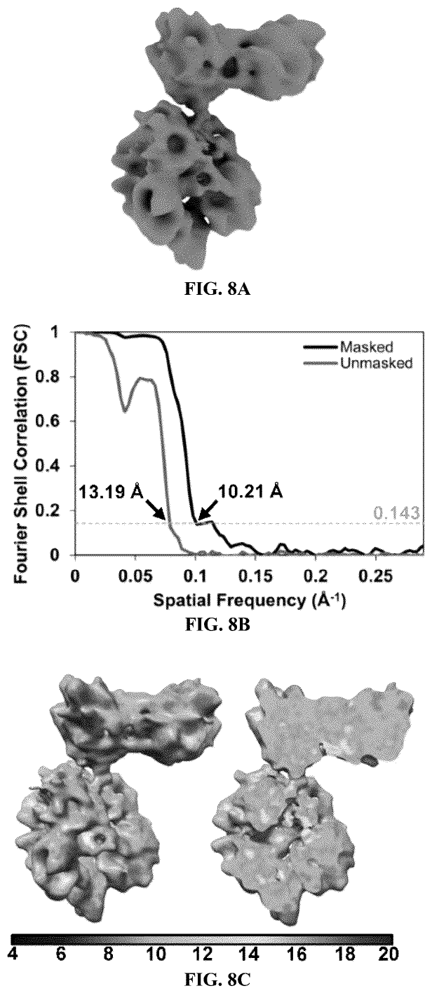

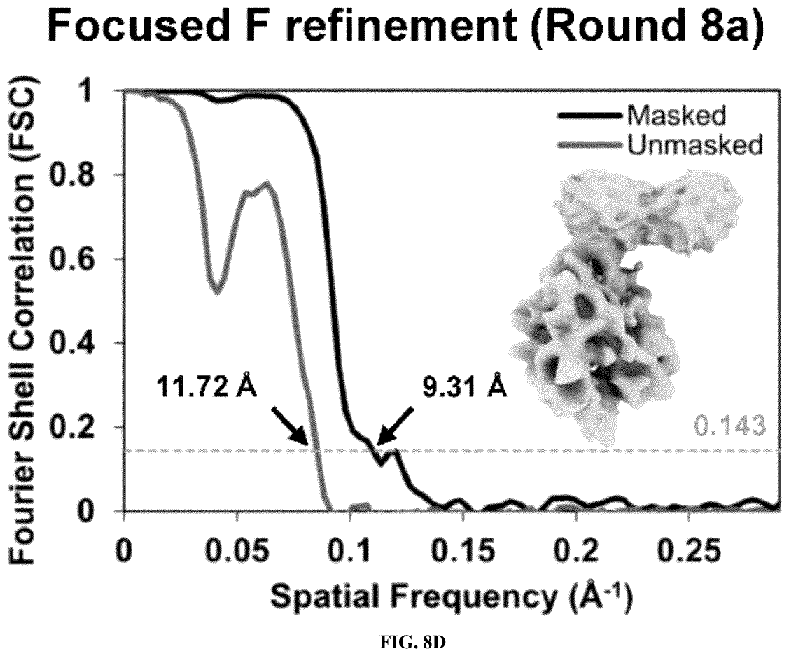

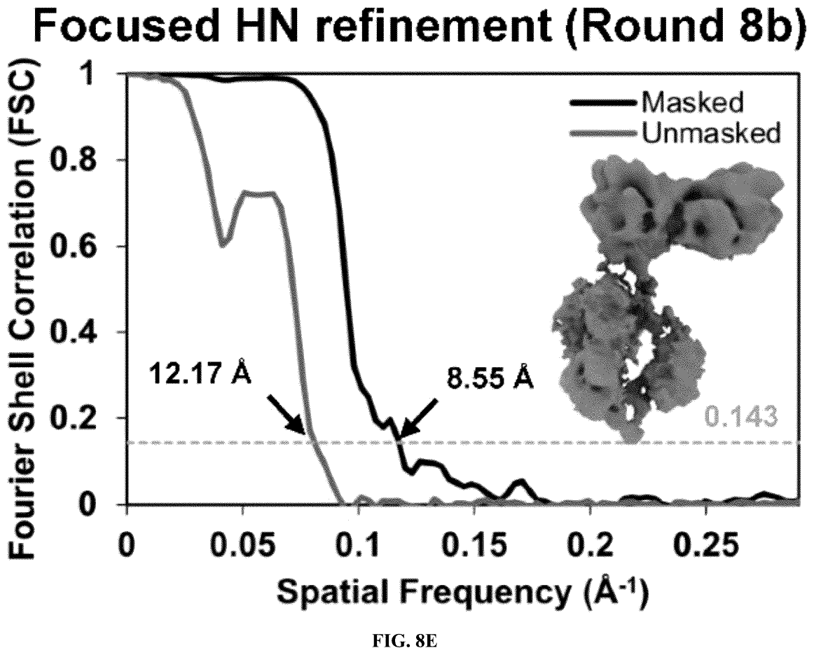

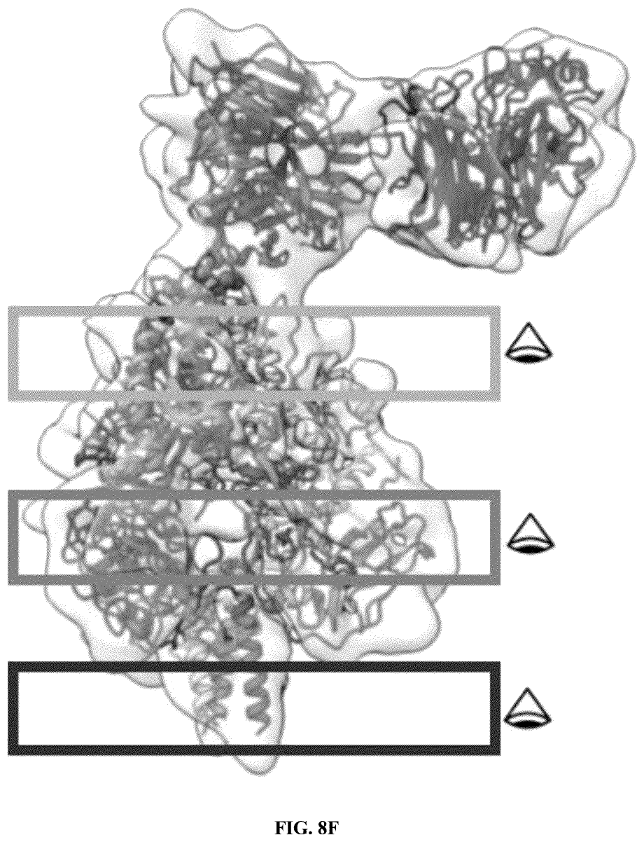

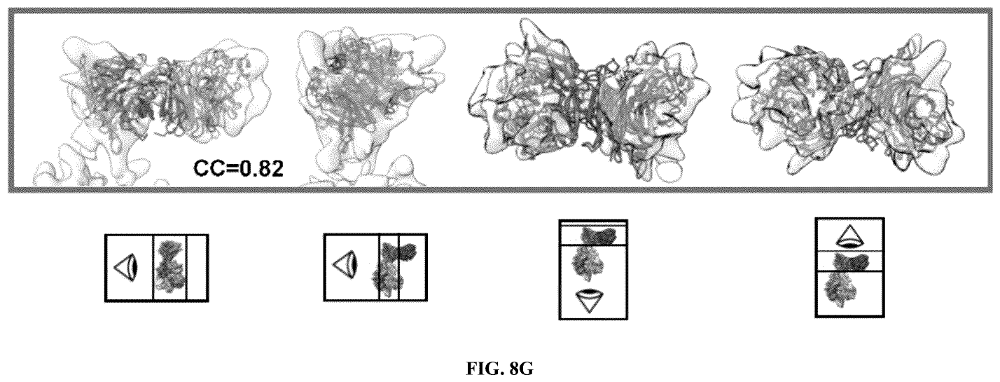

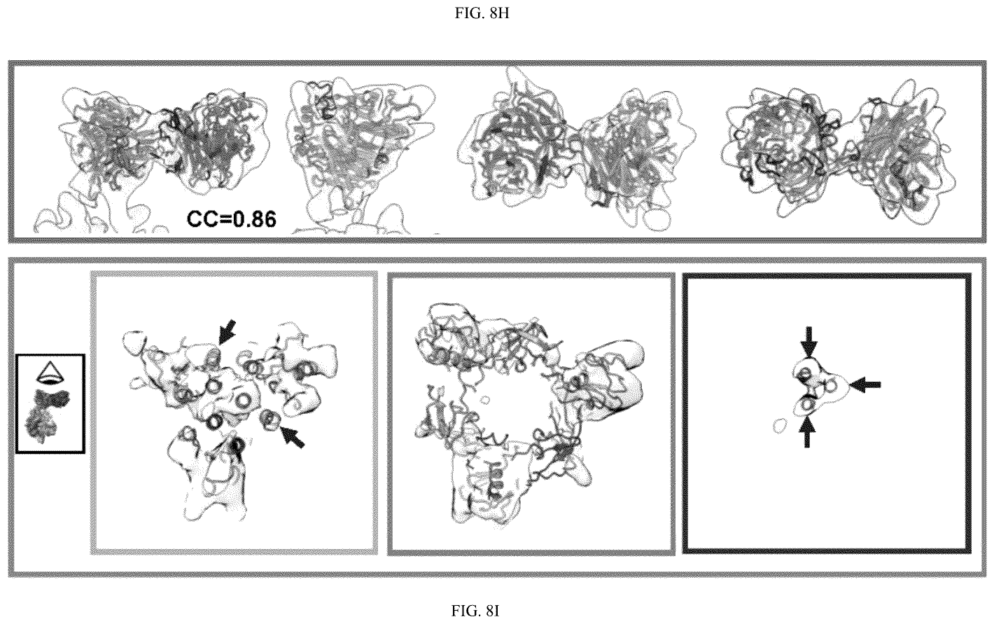

HN and F cover the entire intact viral surface (FIG. 1A) and were used for subtomogram averaging; final subtomogram averages included only those HN and F pairs from multiple viruses with the highest cross correlation (i.e., best fit) compared to the reference structure (FIGS. 1B-1I, 6-8). To maximize the resolution of our subtomogram averages, a tailored processing pipeline that includes adaptation of single-particle strategies [Warp/M] that optimize frame and tilt alignment parameters at the level of each particle was used, so that subtomograms were generated from particles extracted directly from the tilt series rather than from the full tomograms (FIG. 7) and obtained subtomogram averages of the HN/F complex at an overall resolution of 10.2 Å. Further refinement that focused on the HN or F globular heads yielded subtomogram averages at resolutions of 8.5 and 9.3 Å, respectively (FIGS. 1C-1G and 8A-8E). The model derived from the cryo-EM structure of the soluble portion of F [Protein Data Bank (PDB) ID: 6MJZ] was fit into the final density (FIGS. 1F-1G). The model of individual HN protomer heads, which were derived from the crystal structure of the soluble portion of the HN dimer [PDB ID: 4MZA], was also fit into the final density with a model-to-map Fourier shell correlation (FSC) of 9.0 Å (FIGS. 1F-1G and 8F-8I). The fit-to-map cross-correlation values were better for the individually fit HN globular head protomers (0.86) (FIG. 8H) than for the crystal structure of the soluble HN dimeric globular heads (0.82) (FIG. 8G) and showed better fit in the density. The HN dimer heads extend 175 Å above the viral membrane surface, above F (FIGS. 1H and 1I). The long α-helical HN stalk is not observed, most likely due to its flexibility. Therefore, an extended HN dimer derived from AlphaFold structure prediction software was modeled and fit this model into our final subvolume, supporting the notion that the subtomograms are compatible with the complex inserted into the viral membrane (FIG. 1I).

At the final resolution, the head of one of the two HN protomers is observed to interact with the apex of F (FIG. 1F). The side view (FIG. 1F) reveals the orientation of HN in relation to F. One globular head interacts with F, while the other is positioned distal to F and does not interact with F. F's structure is consistent with the previously solved cryo-EM structure of soluble prefusion F. α Helices, while not completely resolved, can be seen in the stalk of F in the density map and in the apex of F (FIG. 8I); FIG. 8I (far right panel) shows the stalk separately as if viewed from above, where the α helix resolution can be better observed. The F apex appears rigid, as suggested by the high local resolution (FIG. 8I). Of note, much of the stalk region of F is resolved in this complex without the need for stabilizing mutations to retain F in its prefusion state as was required for the soluble fusion proteins of related viruses.

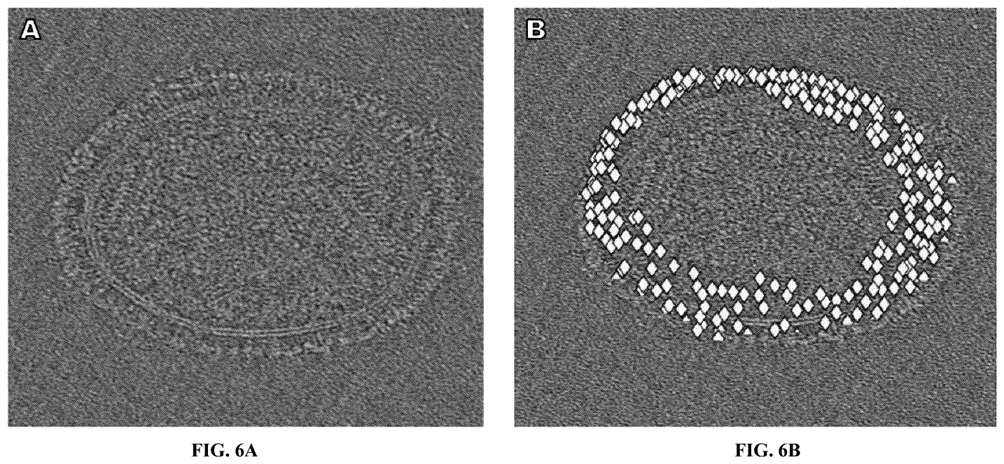

The densities observed on the intact virus surface are consistent with HN forming a dimer, when resolved in its complex with F (FIG. 8H). Evidence is not found for a tetramer or dimer of dimers. To better capture the ultrastructure of the HN canopy, subtomogram averaging and classification of a larger area on the viral surface using a mask encompassing only the HN canopy was performed. Density maps from this classification (FIGS. 1J and 6C) show the level of variability of the HN canopy and lack of defined ultrastructure beyond the HN dimer. This view of the HN canopy further supports that HN is present at the viral surface as dimers that are separated from each other and does not show tetrameric-like densities or evidence of stable interactions beyond the dimer.

b. Relationship Between HN Globular Heads in the Complex

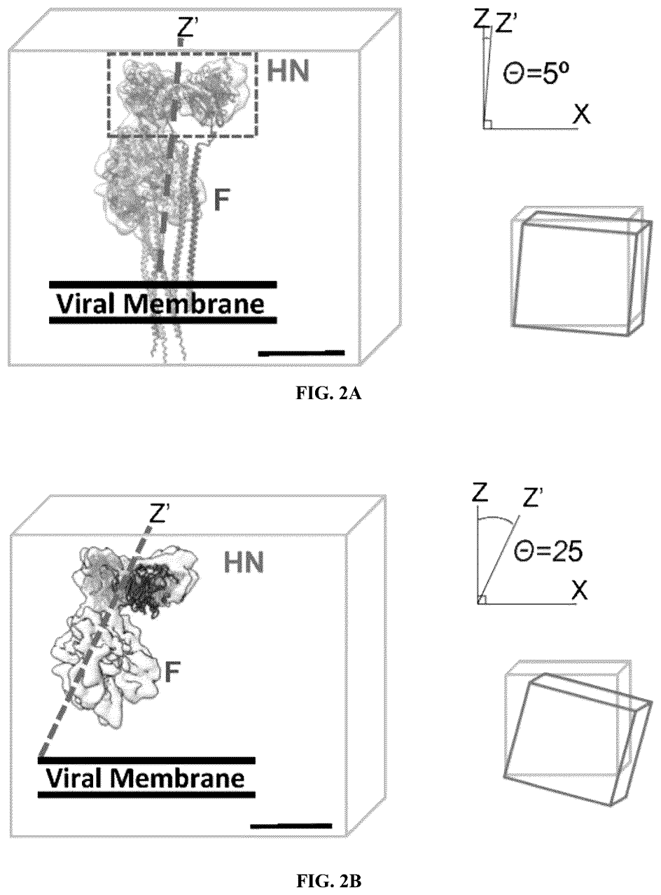

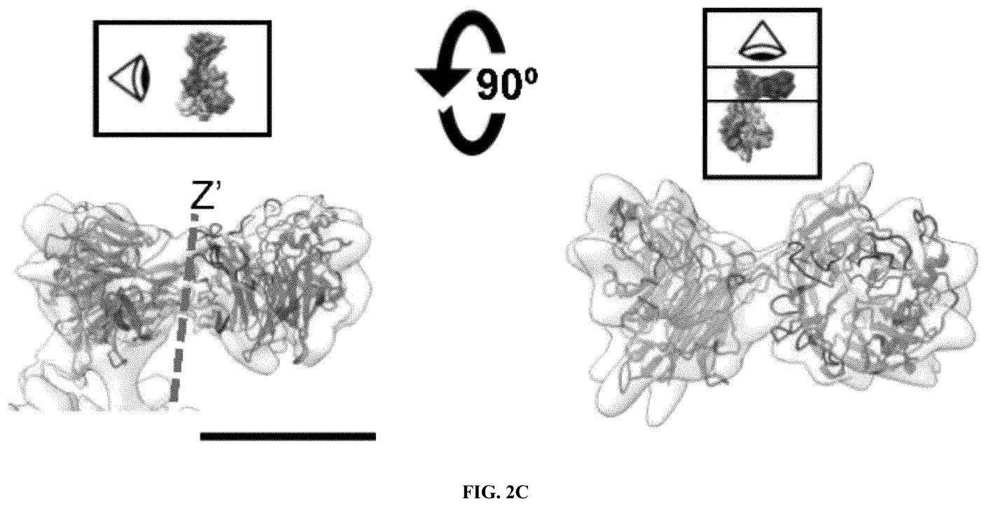

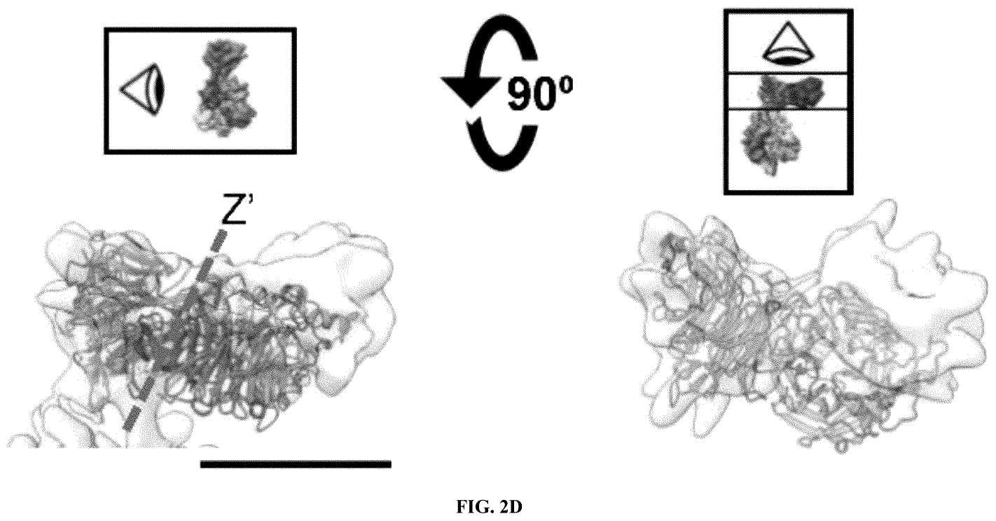

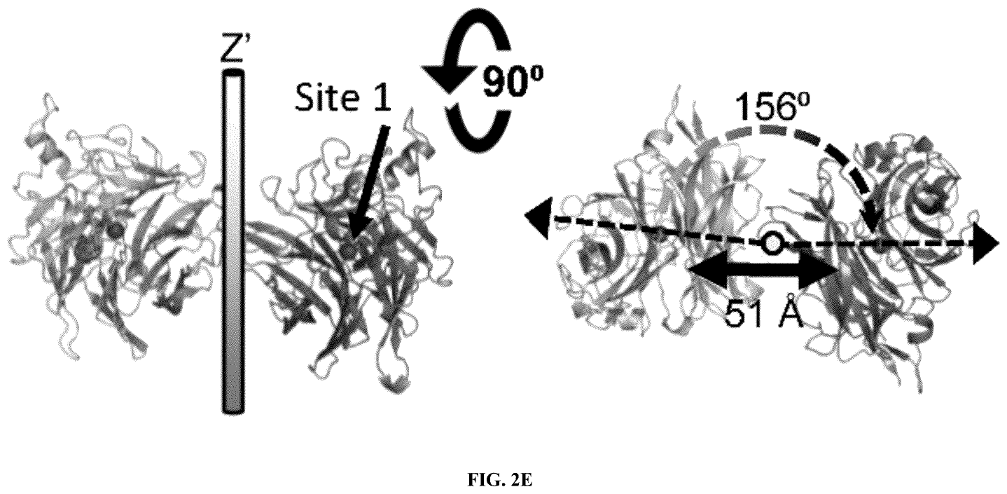

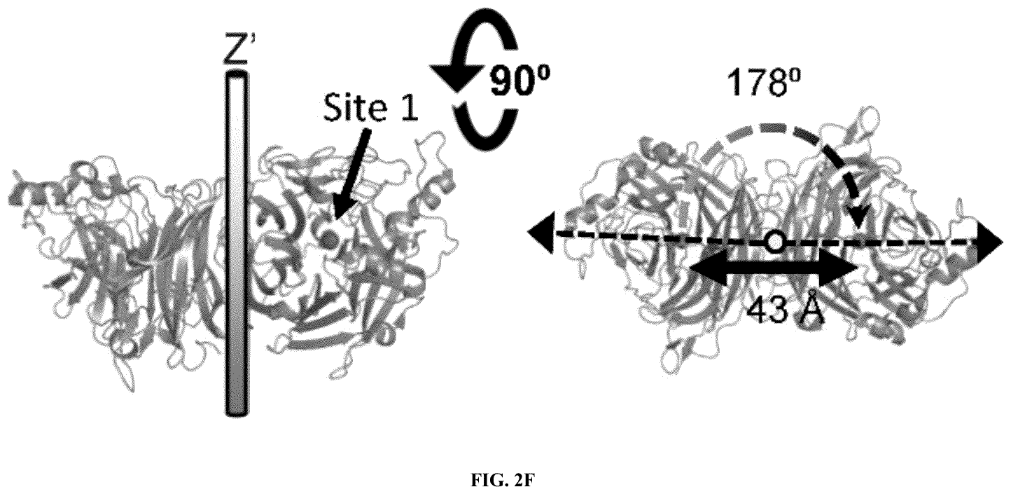

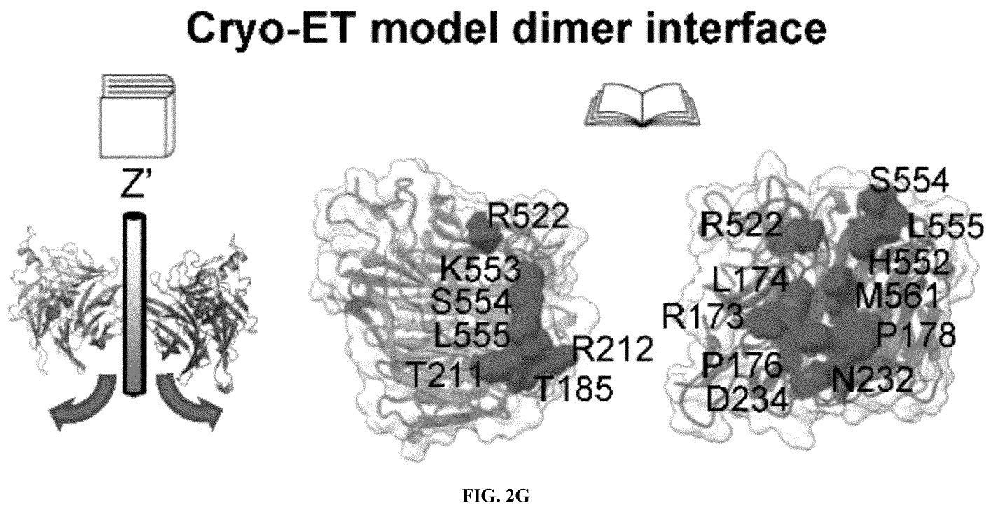

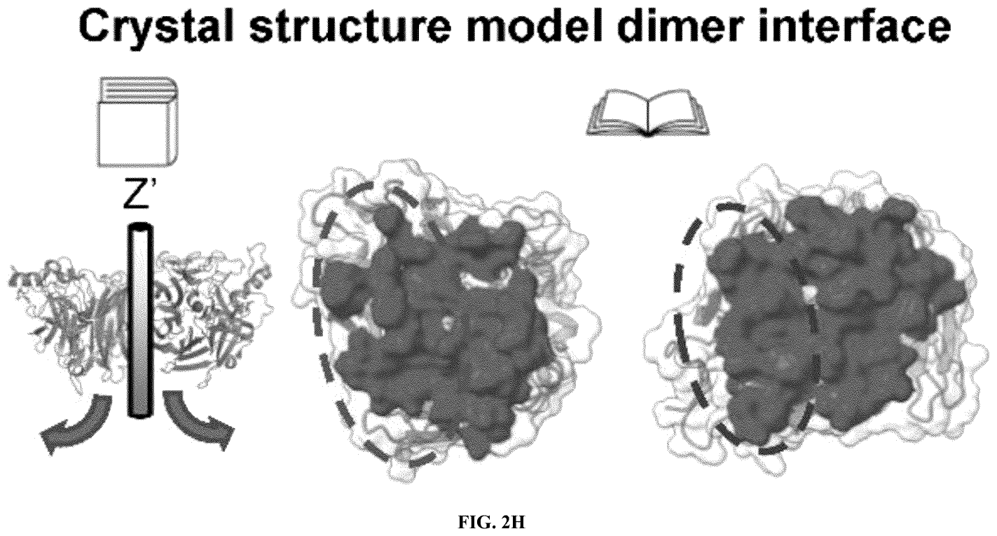

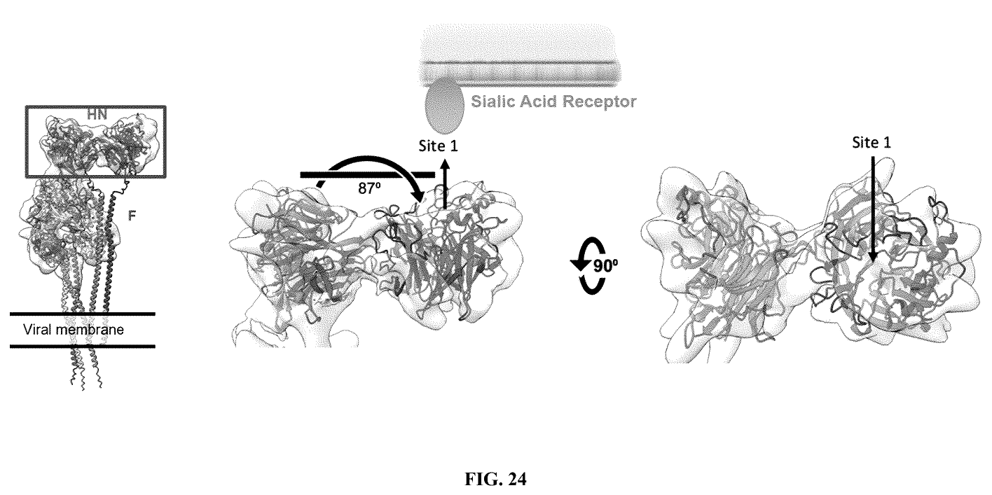

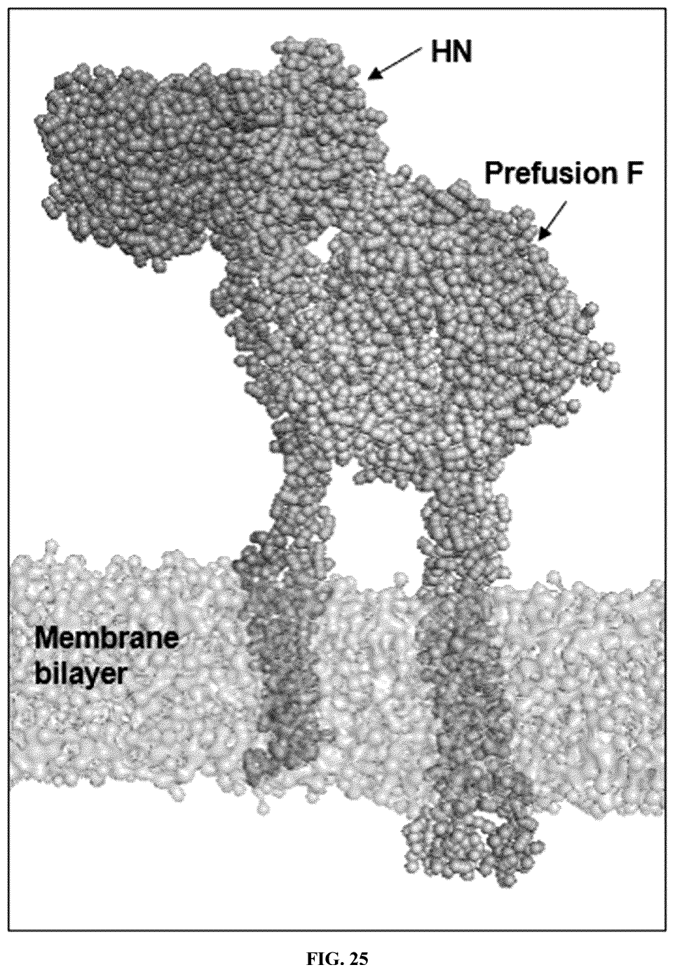

In the HN/F complex, it is observed that, in each HN dimer, the heads are rotated with respect to each other, with one protomer's binding site well positioned to bind the sialic acid receptor, while the other protomer is rotated to interact with and stabilize the prefusion form of F (FIG. 24). The globular head of one HN protomer—the protomer distal to F—is positioned with its primary sialic acid-binding site exposed above the complex and pointing upward, in the direction of a putative receptor (FIGS. 2A and 2E). The globular head of the HN protomer proximal to F is rotated with respect to the other protomer's head (FIGS. 2A, 2C, and 2E), so that its primary sialic acid-binding site near the protomer's center of mass (FIG. 2E, red sphere) is facing orthogonally in relation to the viral membrane in FIG. 2A. This suggests an organization where, in each HN dimer, one protomer interacts with F, while the other is available to bind the sialic acid receptor.

For the cryo-ET model, when the HN protomer head structures are individually fit into the cryo-ET density as in FIG. 2A, the fit and the axis of rotation (Z′) between the protomers is consistent with the angle of the HN/F complex with respect to the viral membrane (90° in the z direction, tilt of 5°) and fits into the density (FIGS. 2C and 8H). However, if the dimeric head crystal structure complex is aligned to the cryo-ET model proximal to F as in FIG. 2B, then the fit and the axis of rotation (Z′) yields a tilt of 25° with respect to an axis perpendicular to the viral membrane (FIG. 2B) and the HN protomer distal to F does not fit into the density (FIG. 2D). In the cryo-ET model, when looking down the Z′ axis of rotation (FIG. 2E, purple bar), there is an angle of 156° between the two HN protomers' heads with respect to the Z′ axis. In contrast, in the crystal structure model, this angle is 178° (FIG. 2F).

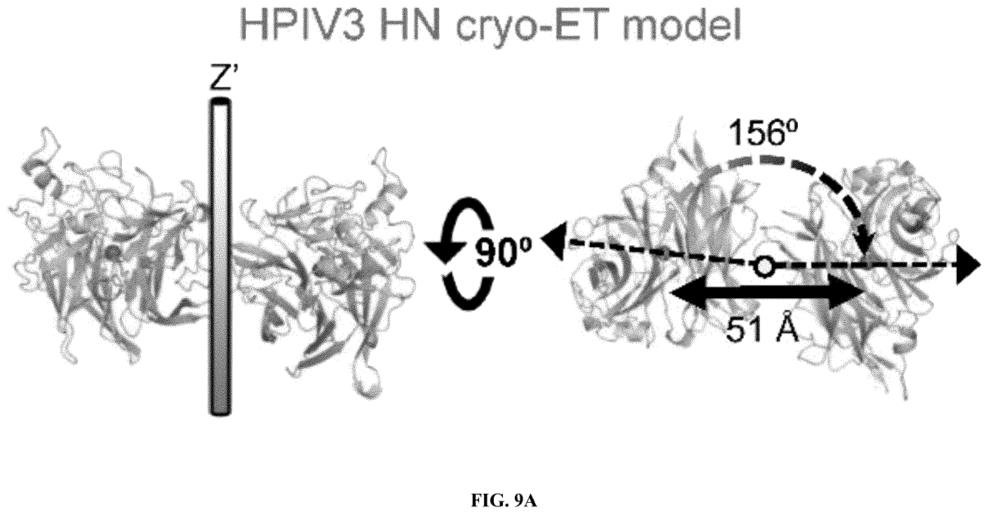

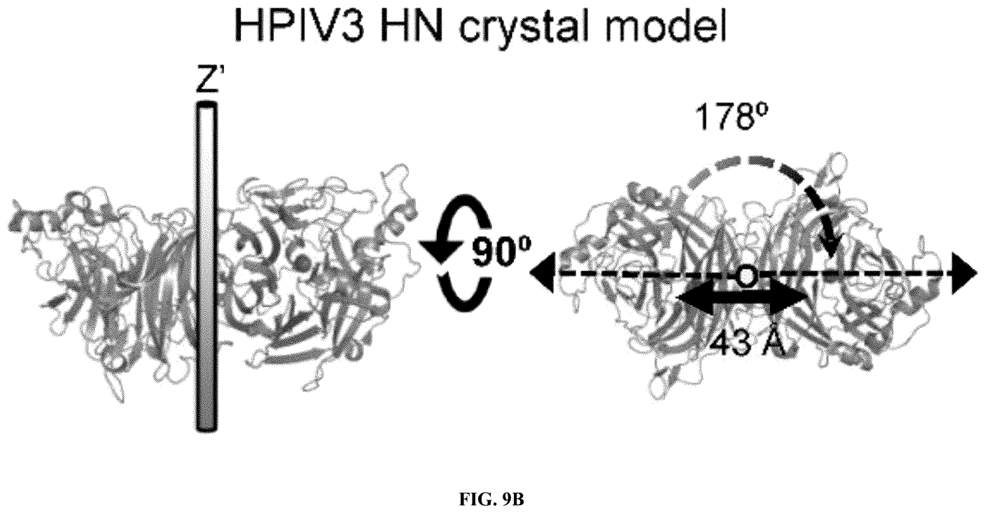

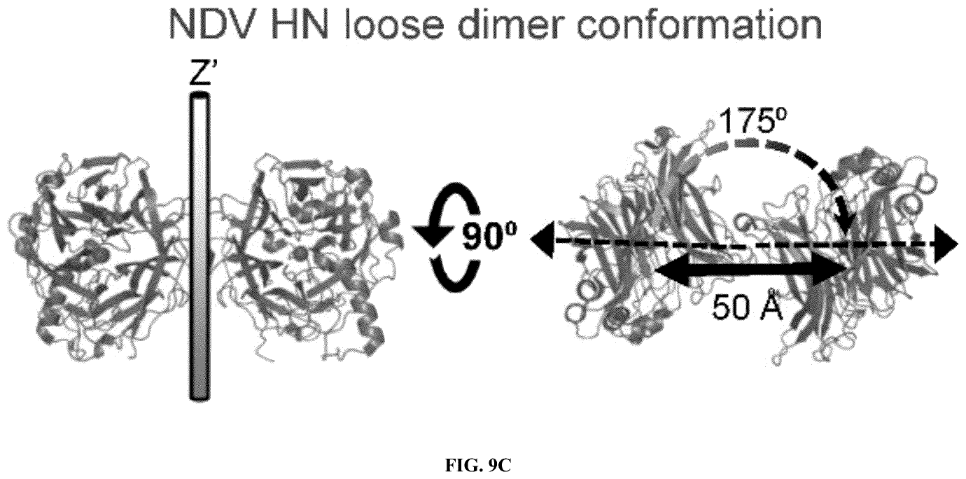

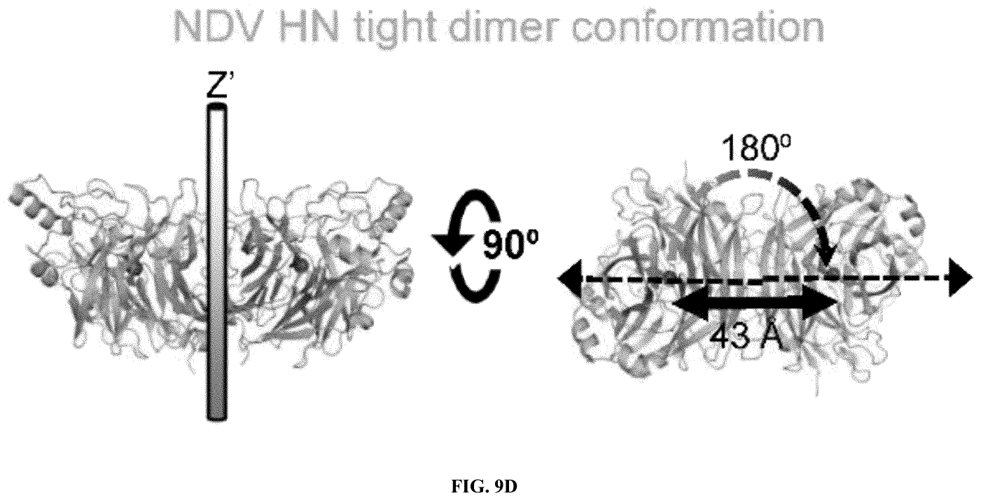

The smaller angle between the protomer heads with respect to the Z′ axis in the cryo-ET structure compared to the crystal structure results in a reduced number of residues (the buried surface area) interacting with the opposing protomer at the dimer interface (FIGS. 2G and 2H) and a longer distance between the centers of mass of each protomer head (51 Å; FIGS. 2E, red spheres) compared to the crystal model (43 Å; FIG. 2F). The heads are in a more relaxed configuration in the cryo-ET model, which we refer to as the “loose” configuration, compared to the “tight” configuration suggested by the crystal structure. A similar difference in HN dimer configuration has been observed for NDV with different crystallization conditions; the low-pH structure shows a loose configuration [PDB ID: 1E8U], while the high-pH configuration shows tight interaction between the heads [PDB ID: 1E8V](FIGS. 9A-9D). Molecular dynamic studies supported the notion that the HN heads can assume these different conformations and rotate with respect to each other.

The HN dimer interface is important for HN's role in activating F and is a key to transmission of the signal for fusion upon receptor engagement. In the crystal structure of the soluble HPIV3 HN dimer, the heads of the protomers are in tight contact at the secondary binding site that forms at the dimer interface and that we have shown to play a role in activating F. We previously showed crystallographically that a mutation in the globular head of HN (Q559R, a residue adjacent to the dimer interface but not contacting the opposing protomer in the dimer interface) relaxes the HN dimer interface in the crystal structure, with a reduced number of residues interacting with the opposing protomer at the dimer interface and showed that this mutation decreases HN/F interaction. This mutation also decreases fusion activation. Several other dimer-interface mutations in HN [HN N551D and HN H552Q] decrease HN's capacity for stabilizing prefusion F and also enhance HN's activation of F. These residues are within the HN dimer interface in both the crystal structure and the cryo-ET structure (FIGS. 2G and 2H). The dimer interface region near residue H552, a residue that we know to be critical for receptor binding and F activation, closely resembles the crystal structure (FIGS. 2G and 2H) and is the region of the interface with least divergence between the soluble crystal structure and our subtomogram average in the complex (FIGS. 2G and 2H). The difference in the angle between the protomer heads in the cryo-ET conformation and the crystal conformation, which alters the residues interacting with the opposing protomer at the dimer interface, raises the possibility that there is freedom of movement between the HN protomer heads.

c. Relationship Between HN and F in the Complex

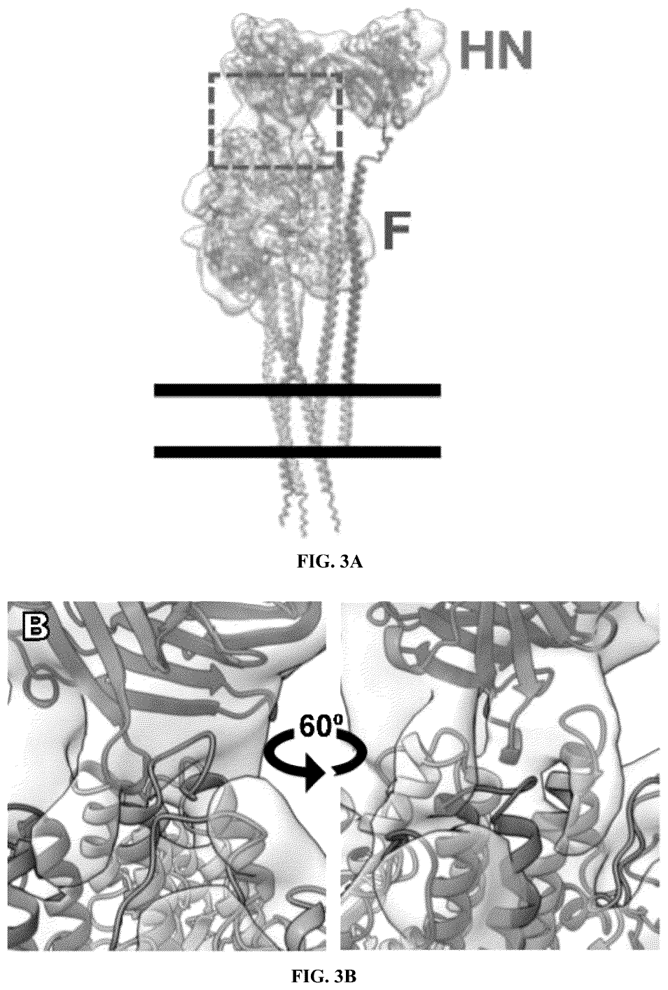

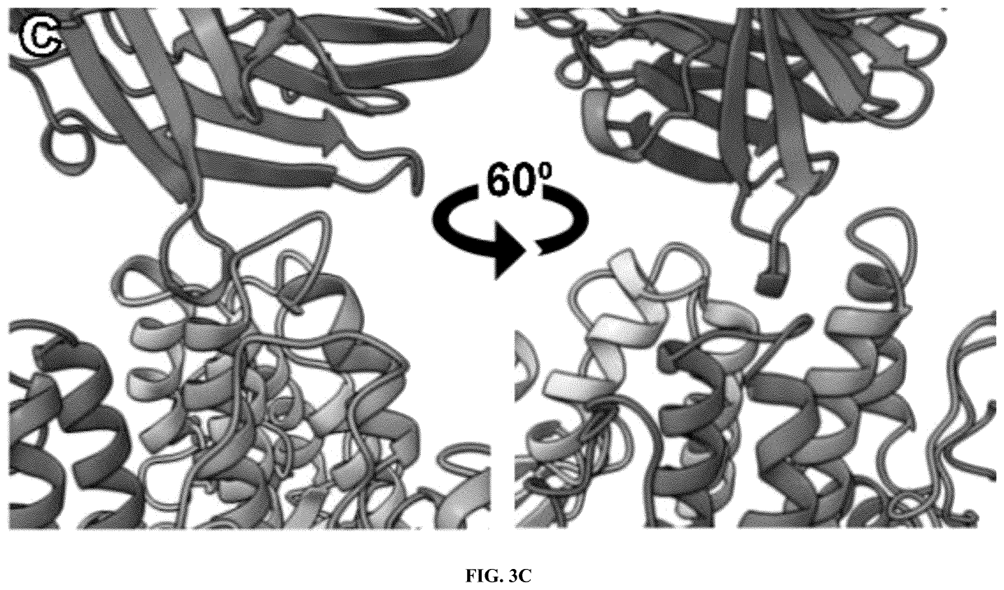

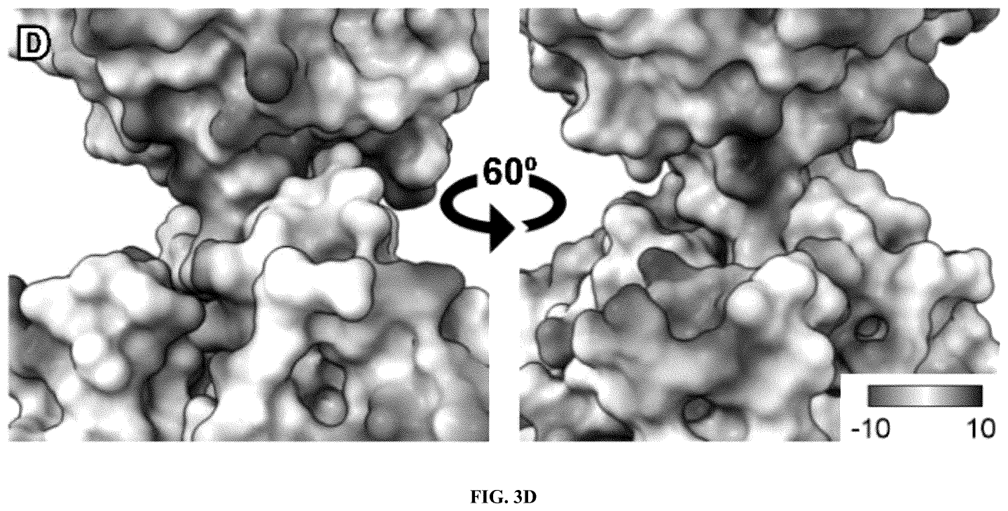

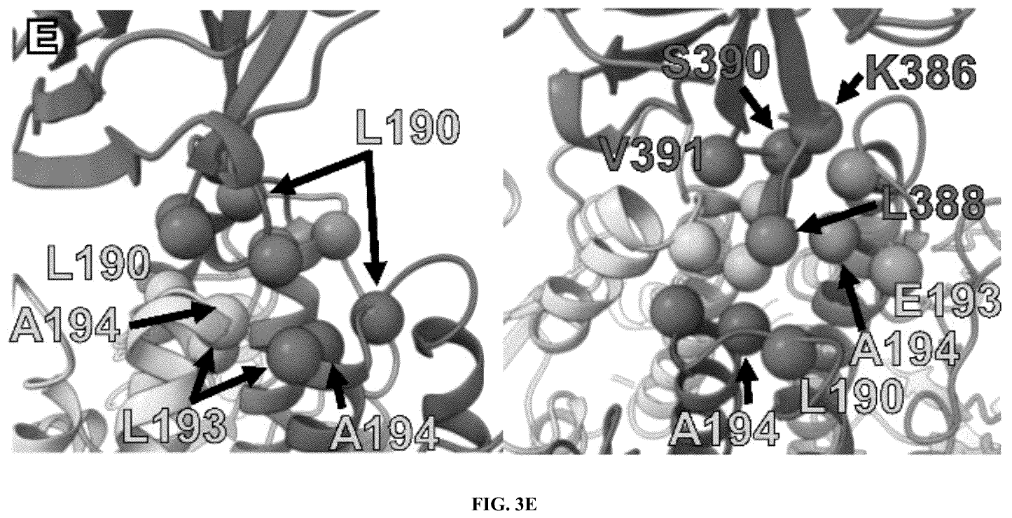

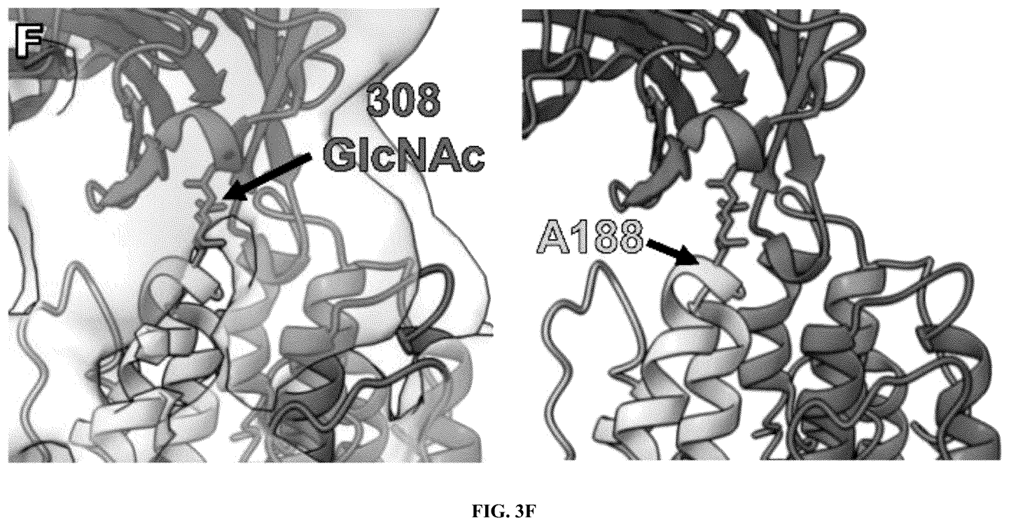

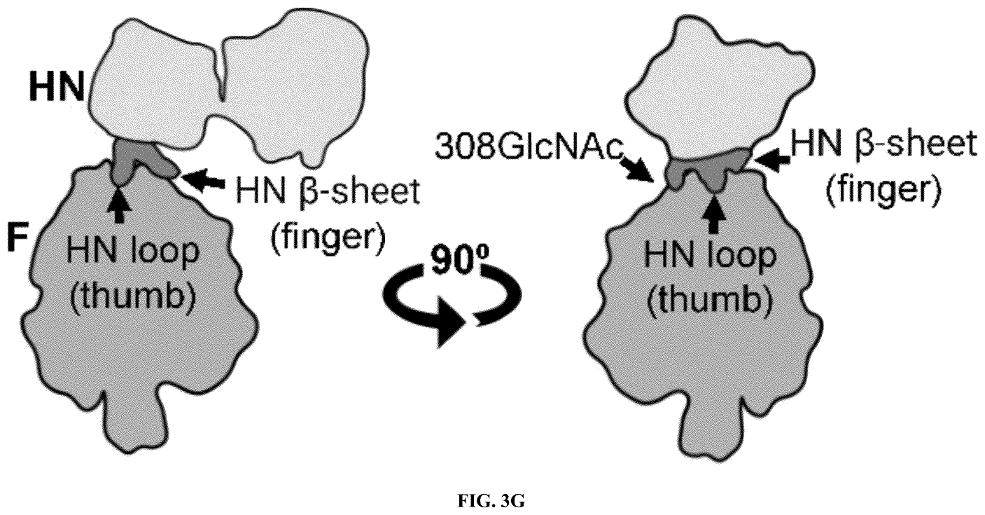

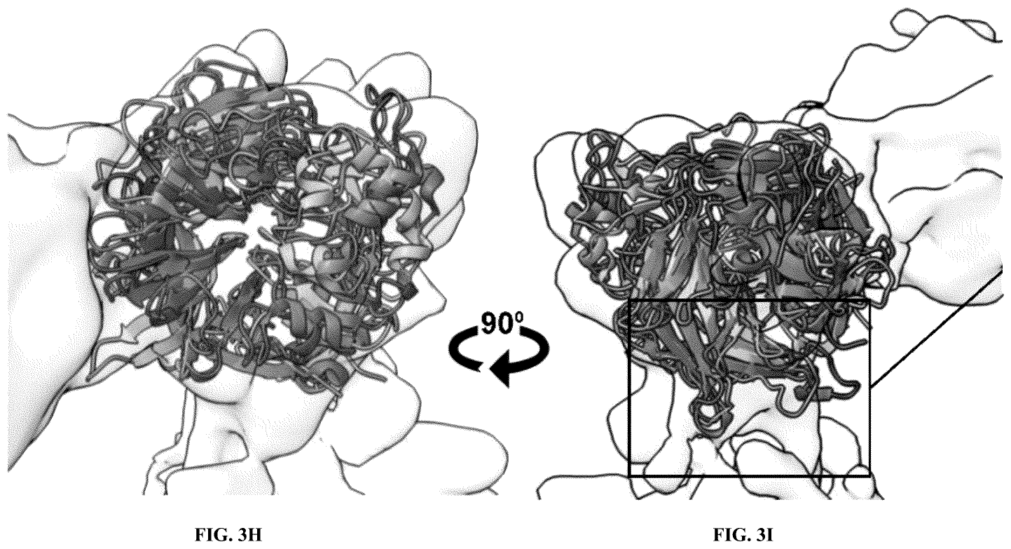

The globular head of the HN protomer directly above F, positioned as shown in the full-length cryo-ET AlphaFold-derived model of HN+F fit into the cryo-ET density (FIG. 3A), contains a loop composed of residues 386 to 392 that extends downward to engage with residues 188 to 196 at the apex of the F trimer. In FIG. 3B, this interaction is placed into the cryo-ET density, and in FIG. 3C, the corresponding cartoon is shown. This 386 to 392 HN loop nestles in the space formed between the protomers of the F trimer, occupying a hydrophobic pocket at the F apex (FIG. 3D). The interaction creates a “thumb-finger” motif in HN facing the F apex, where the “thumb” is the 386 to 392 HN loop, and the “finger” is a R sheet and second loop comprising HN residues 294 to 300 (FIGS. 3B and 3C). Within the HN loop, residues K386, L388, S390, and V391 appear to contact residues in F (FIG. 3E). Additional density observed at the location of glycosylated residue N308 of HN is tentatively attributed to the sugar and extends toward F, suggesting that the sugar may also interact with the apex of F (FIG. 3F). FIG. 3G shows schematically how the HN protomer appears to cap F with these three contact points.

d. Parallel Relationship Between the Receptor Binding and Fusion Protein in Other Paramyxoviruses

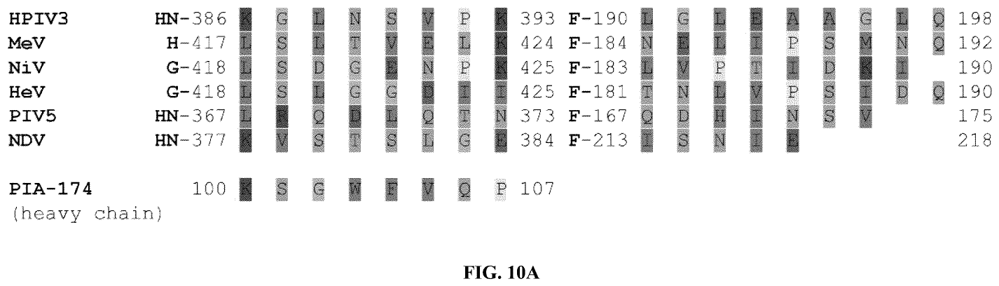

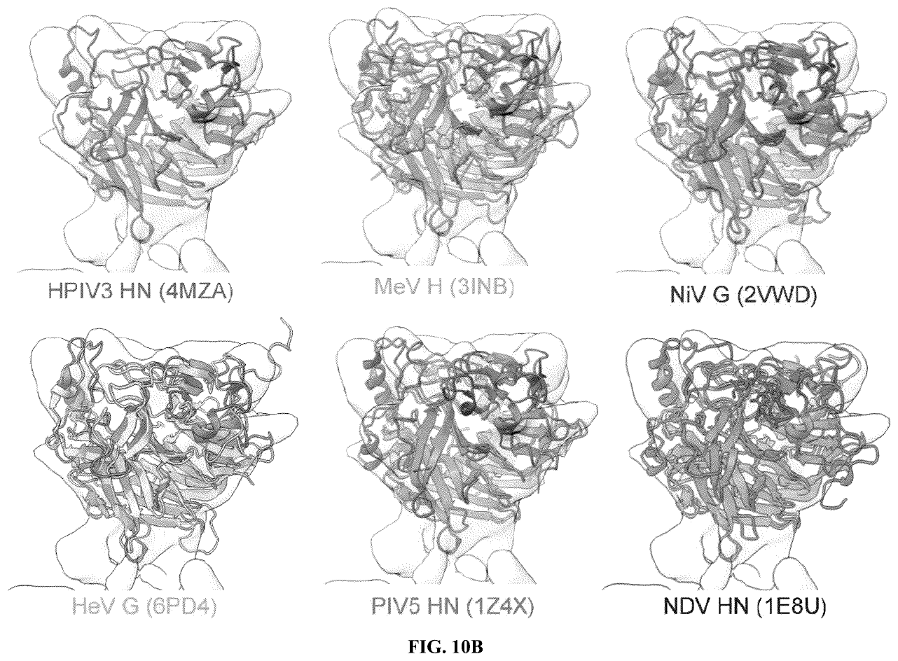

The related paramyxoviruses measles virus and Nipah virus have a HA (H) and G protein, respectively, as receptor binding proteins, and there is no sequence conservation in this domain of the receptor binding proteins. However, overlay of the HPIV3 HN globular heads density with those of measles virus H and Nipah virus G shows notable conservation of structure in this thumb-finger motif (FIGS. 3H to 3J). Despite marked variability in the amino acid sequences of these loops among these viruses (which also includes Hendra virus G, NDV HN, and PIV5 HN; FIG. 10A), the overlay of each structure points to the structural conservation of this loop and of its relationship to F (FIG. 10B).

### Example 2. Target Site for Antiviral Activity

a. Prefusion F Fab Antibody and HN Bind the Same Region at the Apex of F

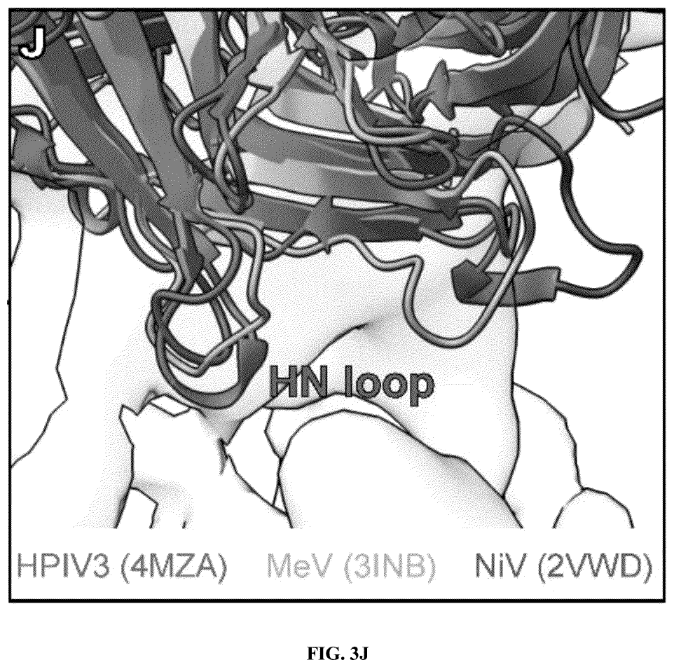

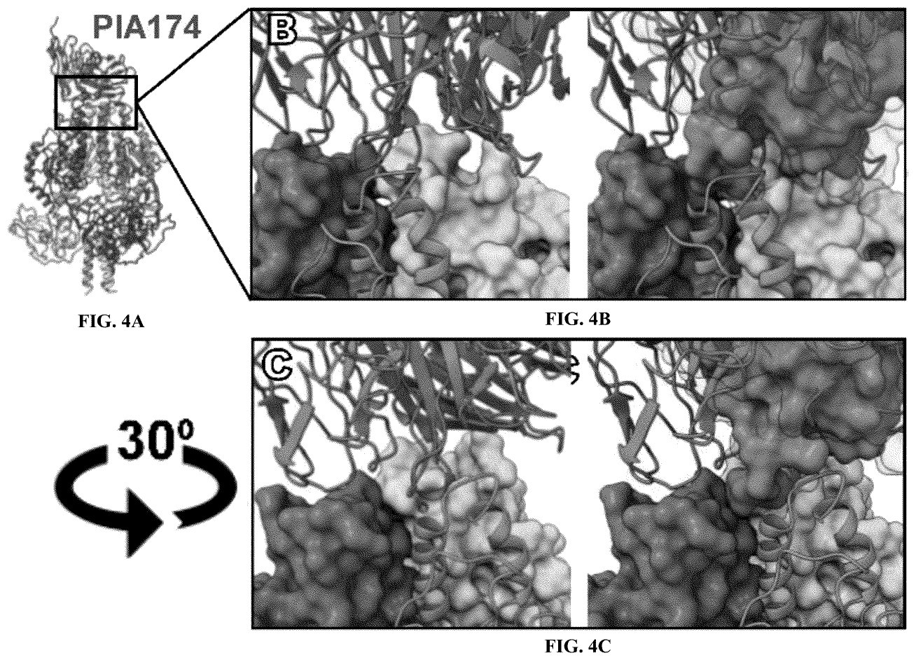

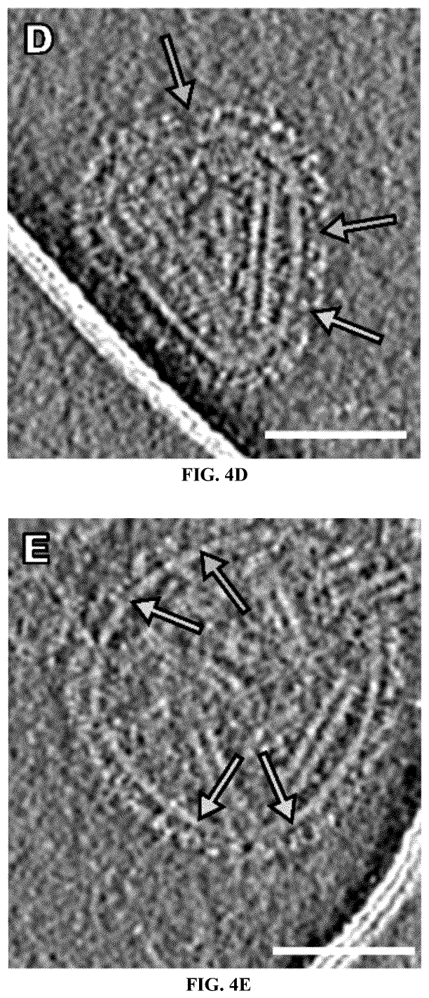

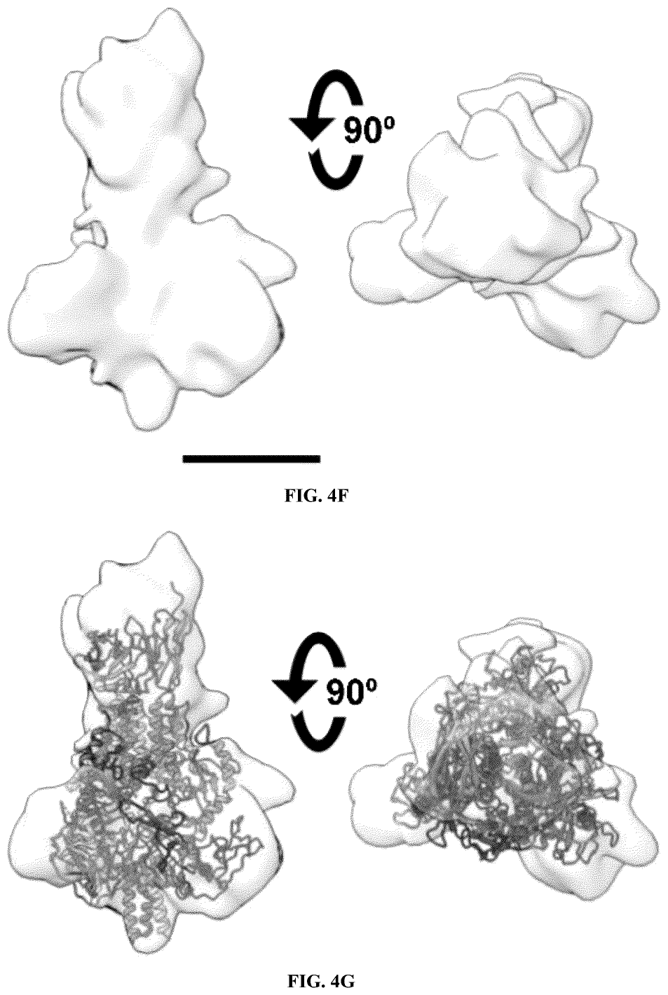

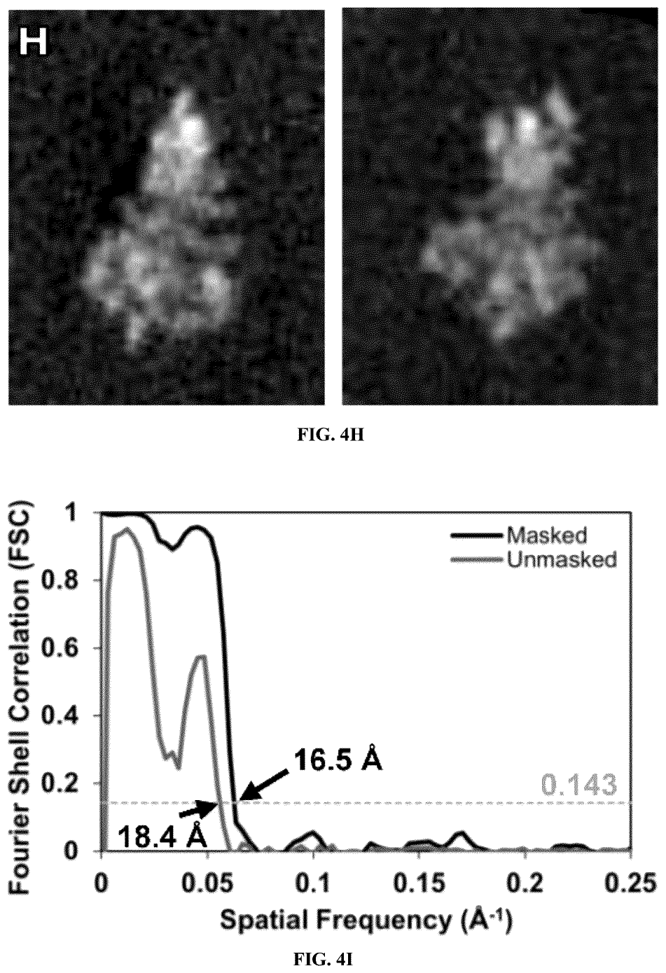

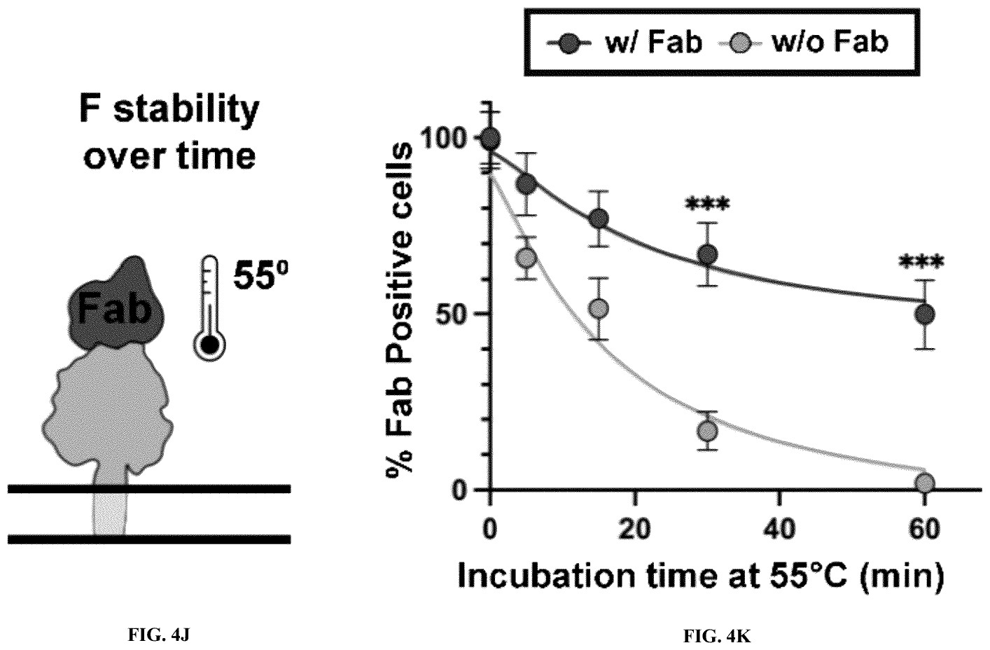

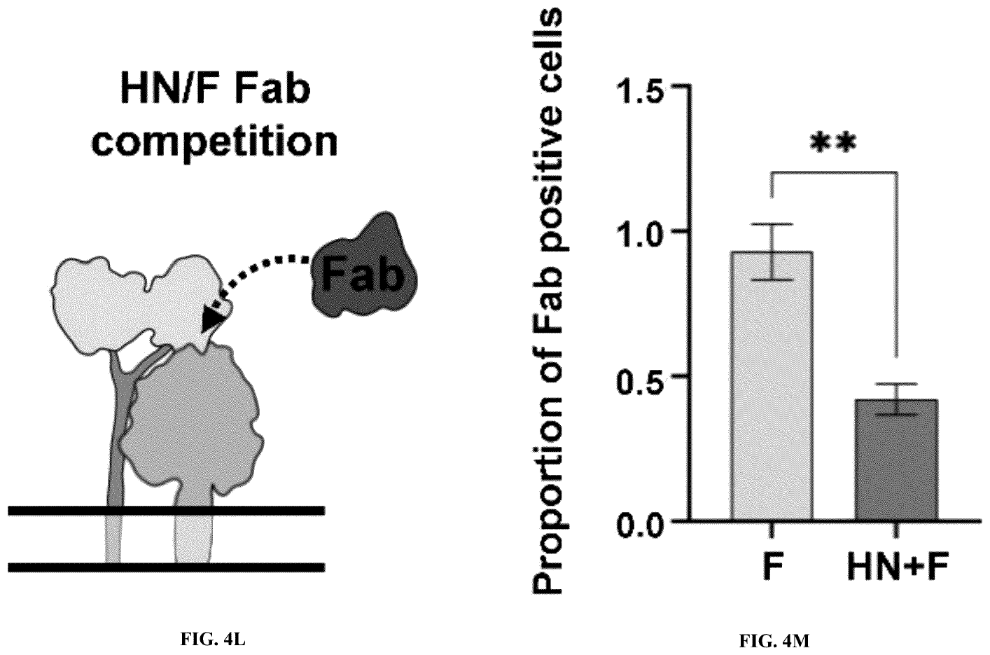

An anti-F neutralizing antibody that inhibits infection (PIA174) binds to the apex of F at the same site as HN in the HN/F complex. The structure of the HPIV3-neutralizing Fab antibody (PIA174) complexed with the stabilized soluble region of prefusion F was solved by cryo-EM. The Fab fragment bound to the apex of F at antigenic site Ø (FIG. 4A). The site on F bound by the PIA174 Fab in this structure precisely overlaps the domain bound by the 386 to 392 HN loop (FIGS. 4B and 4C). Unexpectedly, the Fab antibody fragment contains a very similar thumb-finger motif facing the apex of F as observed in the HN loop, evident in the overlay of the neutralizing Fab antibody atomic surface with the cryo-ET model of HN and F (FIGS. 4A-4C).

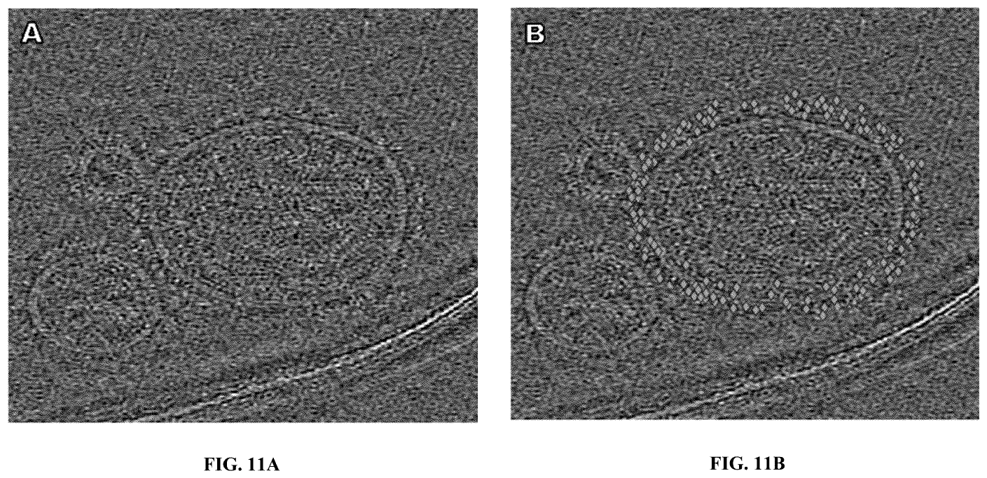

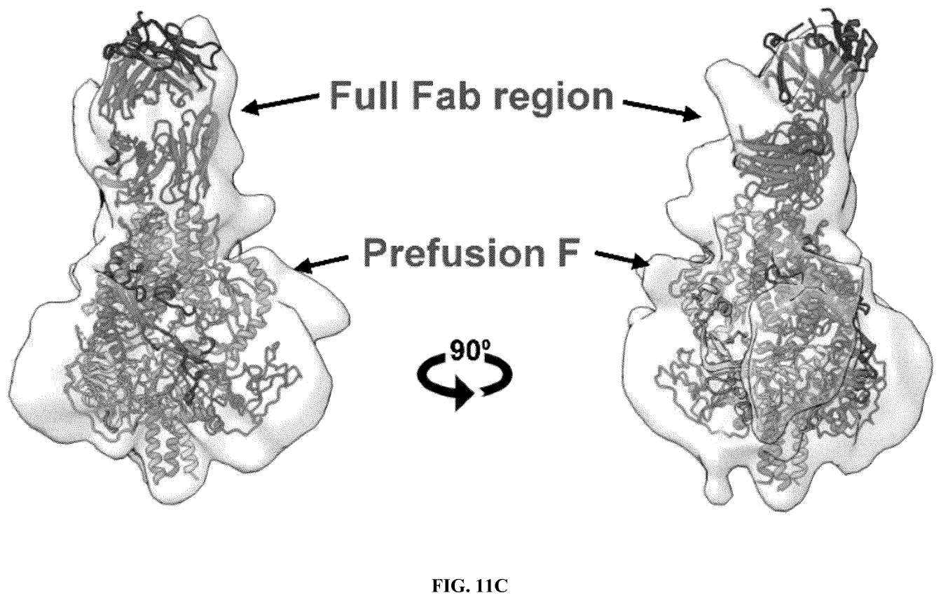

To assess functional overlap between the sites bound by HN and the neutralizing Fab, the PIA174 Fab was added to viruses before vitrification and imaged the resulting sample by cryo-ET. The surface organization of HN and F was altered by the PIA174 Fab with missing surface glycoprotein densities (FIGS. 4D and 4E, orange arrows) and evidence of F glycoprotein present alone (FIGS. 4D and 4E, pink arrows), suggesting a disruption of the HN/F complex (FIGS. 4D and 4E, and 11). Subtomogram averaging of the viral surface on a subset of particles from six of these tomograms reveals prefusion F bound to PIA174 Fab at a 16-Å resolution (FIGS. 4F-4I, 7B 11A, and 11B). The published soluble cryo-EM structure of PIA174-F complex (PDB ID: 6MJZ) fits closely into our subtomogram average density (FIGS. 4F, 4G, and 11C).

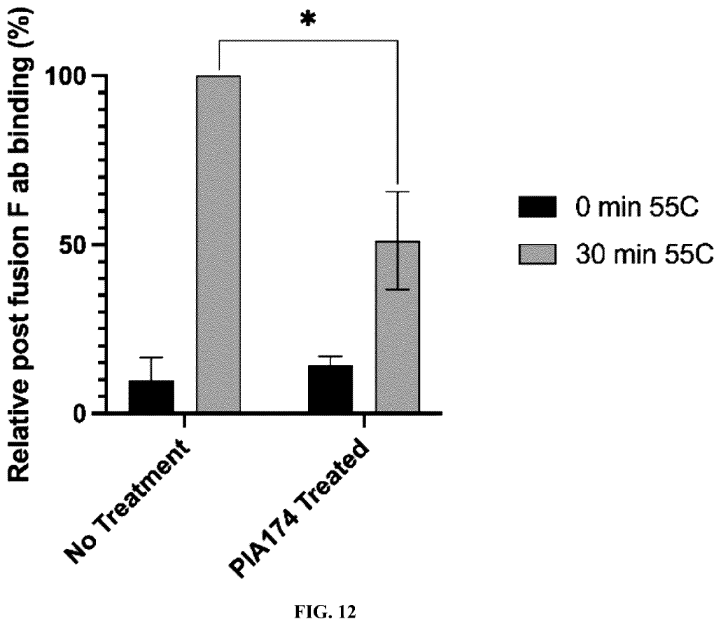

To determine whether the PIA174 Fab stabilizes prefusion F and prevents heat temperature-mediated activation of F at 55° C. in the absence of HN (schematic in FIG. 4J), cells expressing F in the presence or absence of the PIA174 Fab were incubated at 55° C. for varying times. Without PIA174, the proportion of F in its prefusion state rapidly decreases, as all the prefusion F is activated by temperature (FIG. 4K, pink line). However, in the presence of PIA174 added before the onset of heat, F maintains its prefusion state (FIG. 4K, blue line). This observation that PIA174 stabilizes the prefusion F holds when the same experiment is performed with a conformation-specific Ab that recognizes postfusion F (monoclonal antibody PA3/F4) (FIG. 12).

To obtain functional evidence for an overlapping binding domain on prefusion F for HN and PIA174 Fab, it was determined whether HN can mask the epitope of the PIA174 Fab binding site in HN/F complexes on the cell surface (schematic in FIG. 4L). Cells expressing F alone or HN/F were either pretreated or not treated with the cross-linker 3,3′-Dithiobis(sulfosuccinimidyl propionate (DTSSP) and then incubated with PIA174 Fab. PIA174 Fab binding was quantitated (FIG. 4M, bars show proportion of Fab binding after compared to before cross-linking). Fab antibody binding to prefusion F was similar in the absence or presence of DTSSP, as expected (pink bar). However, Fab antibody binding to DTSSP-pretreated prefusion F and HN was decreased, suggesting that HN cross-linked with F masks the PIA174 Fab antibody binding site (green bar).

b. Alterations at Key HN/F Interface Probed with Neutralizing Anti-F Antibody

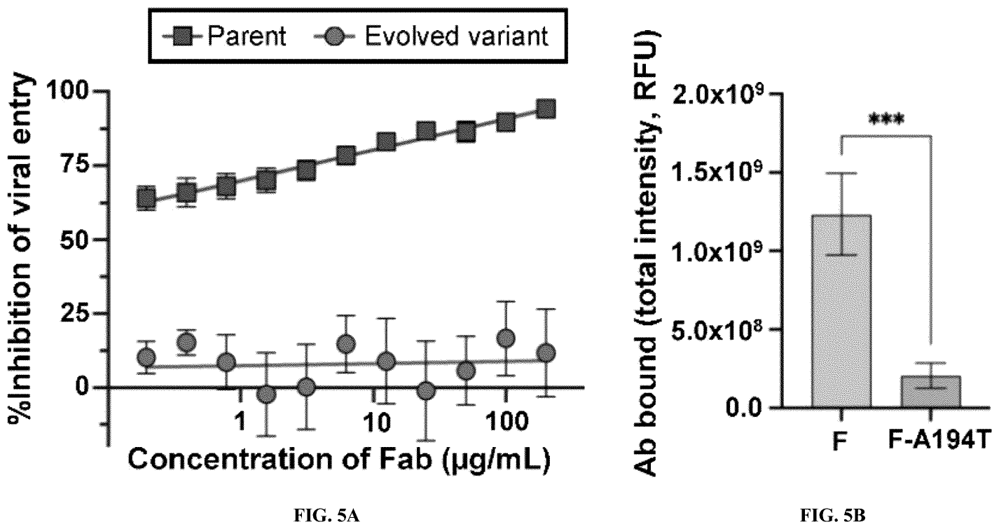

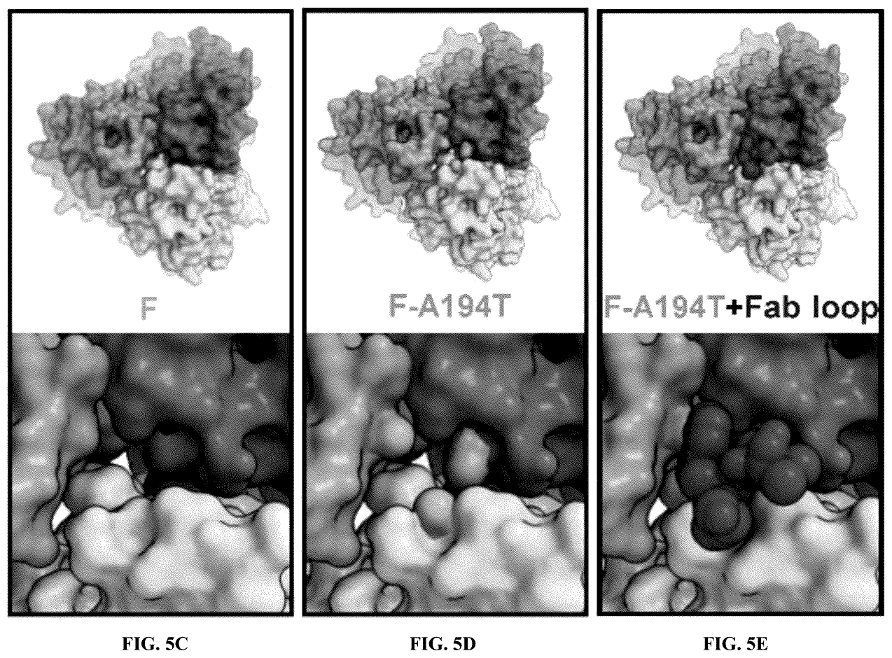

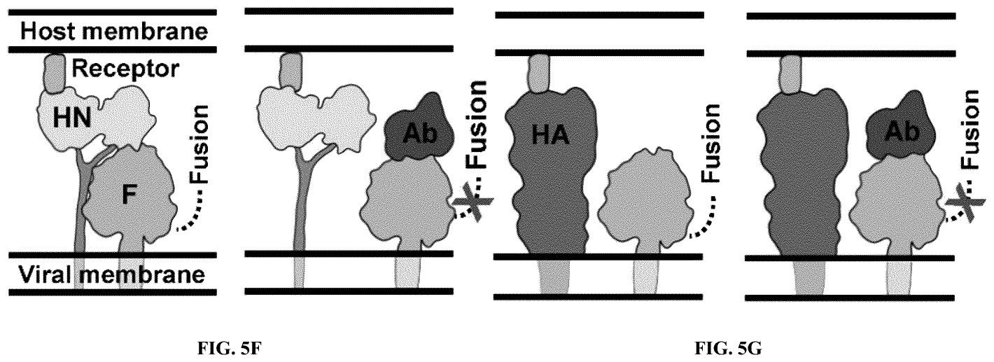

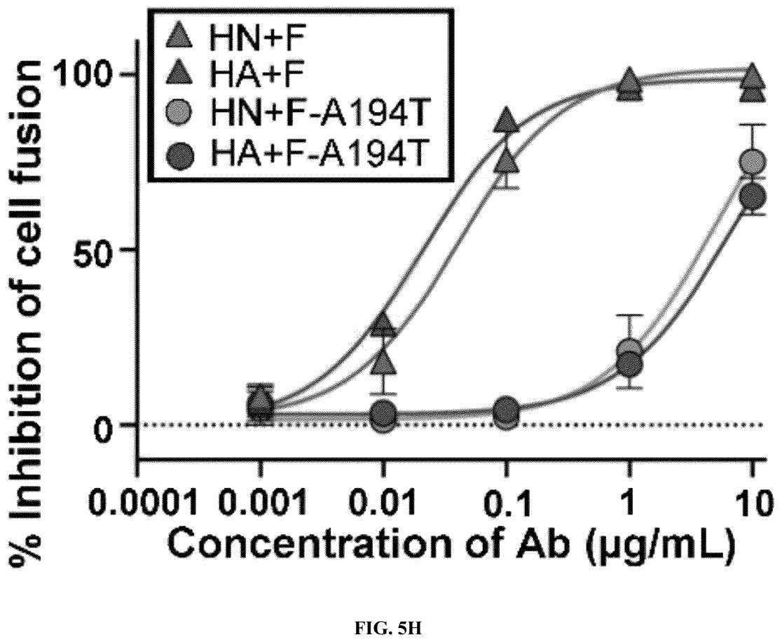

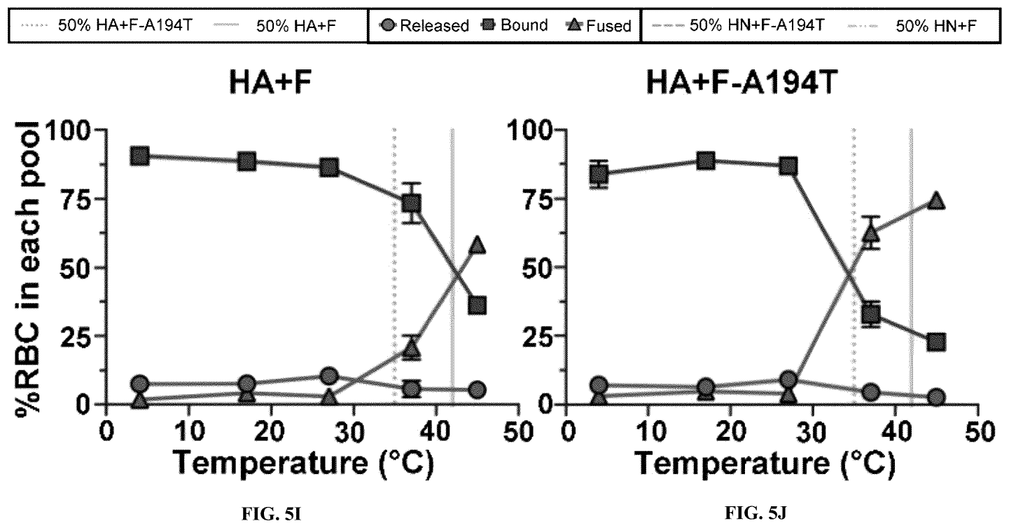

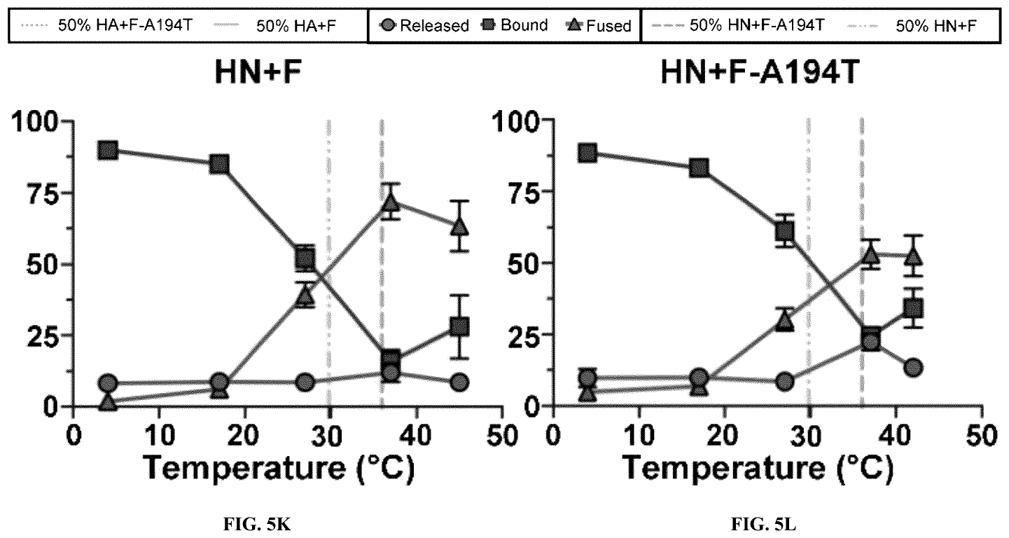

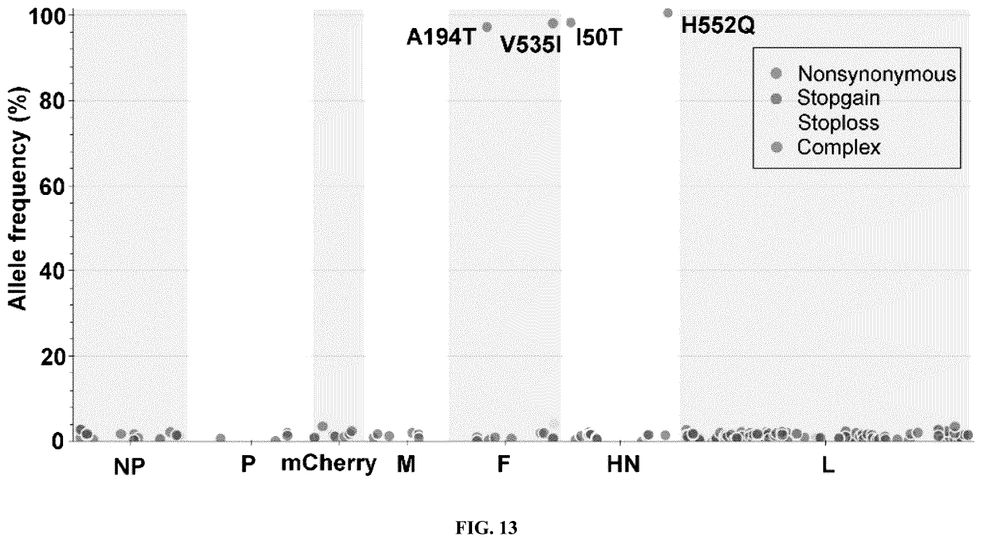

To examine the functional consequences of the overlapping binding epitopes of HN and PIA174 on F, specific mutations derived from both structural and functional information were used. A viral evolution experiment was conducted using the selective pressure of growth of virus in the presence of increasing concentrations of Ab PIA174. Two particularly salient mutations arose in Ab escape variant (FIG. 13); F-A194T at the F apex at the interface between F and Ab PIA174 and HN-H552Q at the HN dimer interface with enhanced F activation properties. The resulting virus was no longer neutralized by Ab PIA174 even at the highest concentrations (FIG. 5A). In expressed F, the A194T mutation decreases PIA174 antibody binding (FIG. 5B). In silico modeling of the F-A194T in the soluble cryo-EM structure shows that the bulkier threonine (T) residue in this pocket may produce steric clash with the Ab loop at this site (FIGS. 5C-5E). To evaluate the effect of A194T on Ab-mediated inhibition of fusion, inhibition of F-mediated fusion by PIA174 Ab in a quantitative cell-cell fusion assay using cells coexpressing F (parental and A194T) and HN (parental) or influenza HA was used, which provides membrane tethering via sialic acid receptor engagement but does not complex with, or activate, F (FIGS. 5F and 5G). The parental F, when paired with either HN (green triangles) or HA (red triangles) was inhibited by PIA174 Ab. The F-A194T when paired with either HN (orange circles) or HA (purple circles) was resistant to inhibition by the PIA174 Ab (FIG. 5H). The resistance to PIA174 Ab inhibition of A194T F-mediated fusion (FIG. 5H) is less than the resistance to the PIA174 Fab demonstrated by the escape variant virus bearing the full complement of four escape mutations in HN and F (FIG. 13).

The impact of the A194T mutation in F on the ability of F to be activated by heat or by HN was evaluated, using an assay that distinguishes between different states of F activation. The readout for F activation is fusion of red blood cells (RBCs) with the F-expressing cells. Cells coexpressing F and HN or HA were allowed to bind to their sialic acid receptors on RBCs at 4° C. and transferred to a range of temperatures to permit F activation. At each temperature, the percentage of target RBCs that were either released into the medium (blue circles), bound but had not fused, indicating that they were either bound by HN/HA (red square) or had undergone fusion (green triangle) were measured. The F A194T is more readily triggered by heat compared to the parental F (FIGS. 5I and 5J; compare 50% fusion temperature for HA+F A194T indicated by orange dotted line to 50% fusion temperature for HA+parental F indicated by pink solid line). The F A194T is less readily activated in the presence of HN (FIGS. 5K and 5L; compare 50% fusion temperature for HN+F A194T indicated by orange dashed line to 50% fusion temperature for HN+parental F indicated by pink dashed line), suggesting that the HN/F interaction has been perturbed. The findings that viral evolution under selective pressure of PIA174 Ab led to a mutation at the precise site that predicted to be the site of HN/F interaction and that this mutation alters both PIA174 Ab binding and HN's activation of F support the notion that this HN/F interaction site represents an important interface between HN and F and functionally overlaps with the site of PIA174-neutralizing antibody binding. Together, the results in FIGS. 4A-4M and 5A-5L suggest that the neutralizing Ab PIA174 inhibits fusion by stabilizing the prefusion state of F and competing with HN and thereby inhibiting F activation.

### Example 3. Importance of HN-F Interaction Site

a. Resistance Properties of Antibody Escape Variants

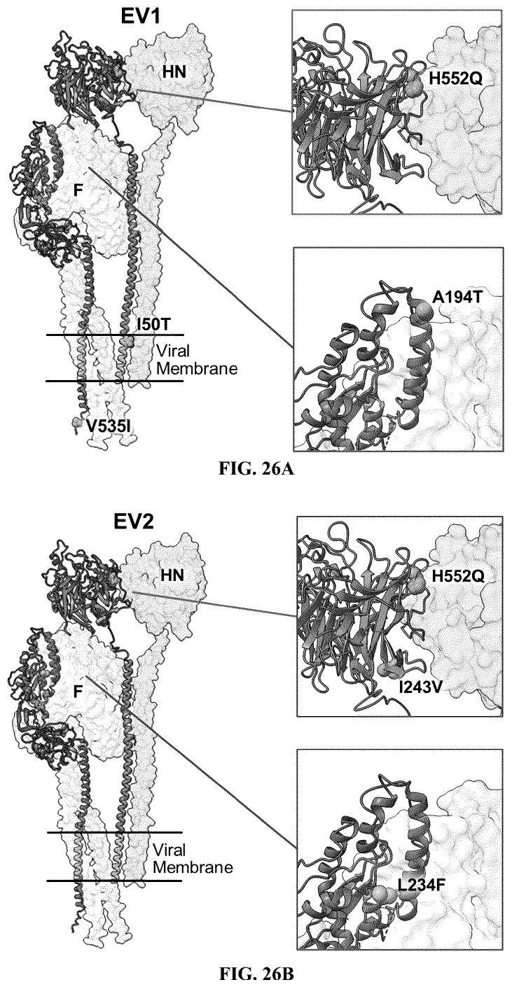

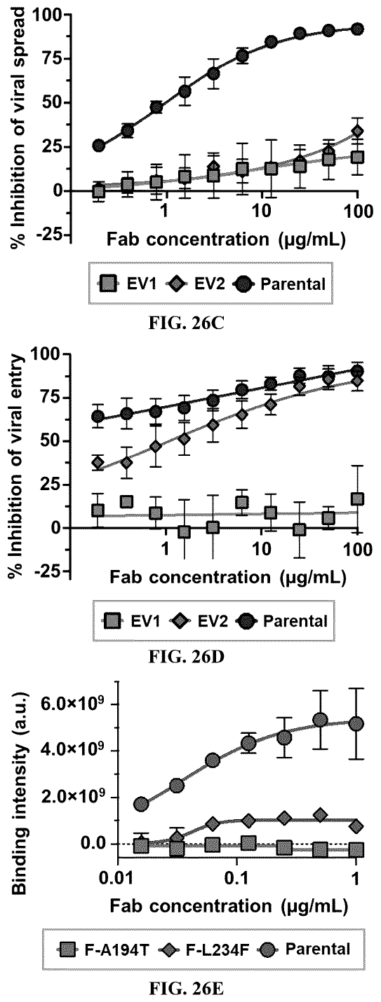

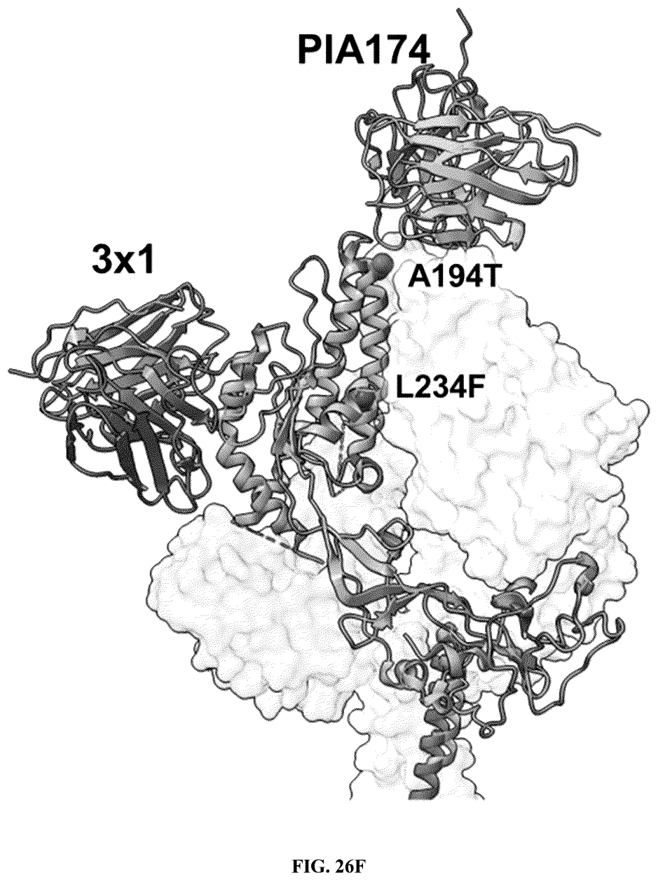

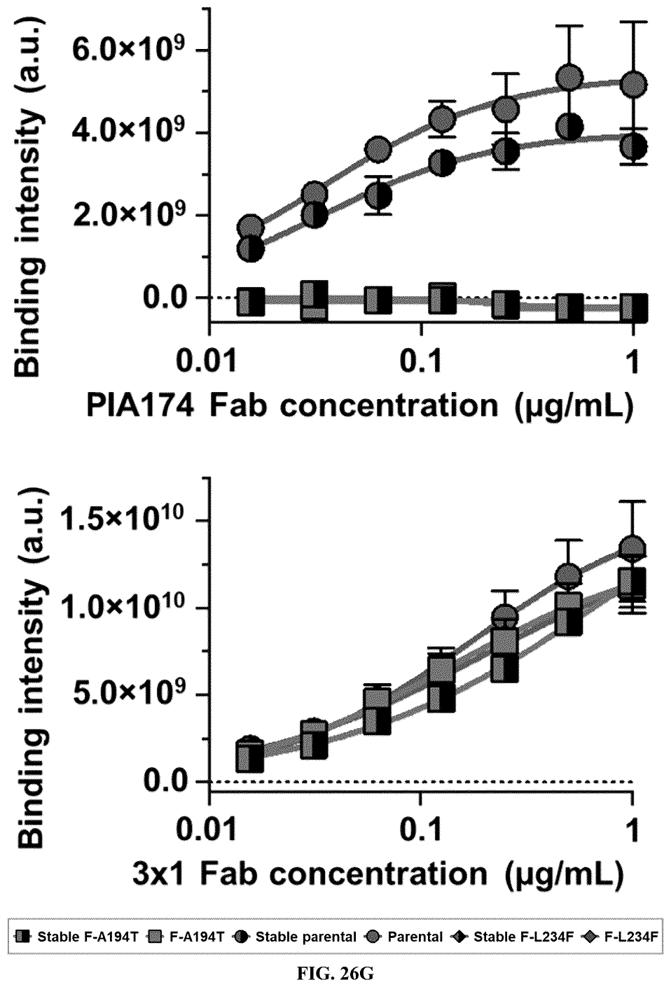

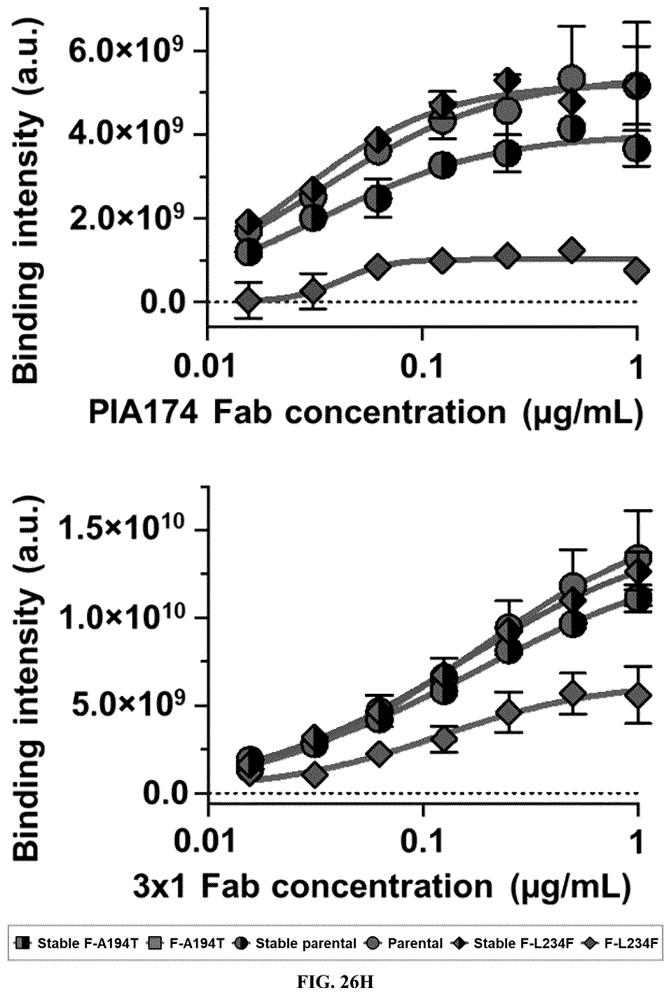

Viral evolution experiments were conducted by using a previously-characterized HPIV3 neutralizing monoclonal antibody PIA174Ab to select for variants that produce infectious virus in the presence of the antibody. Two escape variant (EV) viruses emerged (FIGS. 26A, 26B). One that was previously characterized functionally (EV-1) bears a mutation at the precise site on the F apex that the complex's structure predicts to be the site of HN/F interaction, A194T. This mutation alters both PIA174Ab binding and HN's activation of F. The second PIA174 escape mutant bears a mutation F-L234F at the side of F, distant from the trimeric F apex but adjacent to the central helix of the F trimer (FIG. 26B). Both viruses also bear a mutation at the HN dimer interface (H552Q) that has been previously shown is a mutation that confers increased HN-F interaction and F activation in persistent HPIV3 variants. This PIA174 Fab-escape variant, called “escape variant (EV)-2” to distinguish it from the F trimer apex/HN dimer interface variant (EV-1), like EV-1, spreads throughout a cell culture monolayer in the presence of high concentrations of Ab PIA174 (FIG. 26C). However, the initial viral entry, as measured by plaque formation (FIG. 26D) is inhibited for EV-2 in distinction to EV-1 which is completely resistant to inhibition of entry.

b. Escape Variant Fs Evade Ab Binding

Both the A194T and the L234F mutations decrease PIA174 antibody binding to expressed F (FIG. 26E), a surprising finding in light of the distance of L234F from the F trimer apex binding site of PIA174 Fab. To assess whether the L234F mutation destabilizes the F central helix and thereby alters the PIA174 antibody binding epitope, Ab binding to F-A194T, F-L234F, and parental F with Ab binding to disulfide-stabilized versions of these proteins was compared (FIG. 26F). A194T and L234F mutations were introduced in a stabilized prefusion F that had been used to generate both PIA174 and another known anti-HPIV3 F-specific neutralizing antibody, 3x1. The engineered stabilized F bears 4 cysteine mutations; one pair that connect the central helix (and A194) to the domain III helix-turn-helix that contains the L234F mutation, and another pair in the HRN domain containing the 3x1 Ab epitope. The engineered F bearing cysteines substituted for residues 1213 and G230 staple the central helix domain of the structure and reduce flexibility conferred by L234F, and cysteines substituted for Q162 and L168 staple the HRN domain of the structure. It was probed whether the stabilizing mutations could compensate for the EV1 and EV2 mutations and restore PIA174 Fab binding. FIG. 26G shows that while PIA174 Fab binds the stabilized parental F, it fails to bind the cysteine-stabilized F-A194T. However, PIA174 Fab binding is fully restored to the stabilized F-L234F (FIG. 26H). As expected, Ab 3x1 binds equally to all three stabilized Fs and is unaffected by either A194T or L234F (FIGS. 26G and 26H). These results suggest that the escape mutation L234F, distant from the F trimer apex, nonetheless destabilizes the F trimer interface thereby perturbing the trimer apex PIA174 antibody epitope.

c. Resistance Mutations Alter HN-F Complex Function so that F More Readily Activates but has a Defect in Completing the Fusion Process

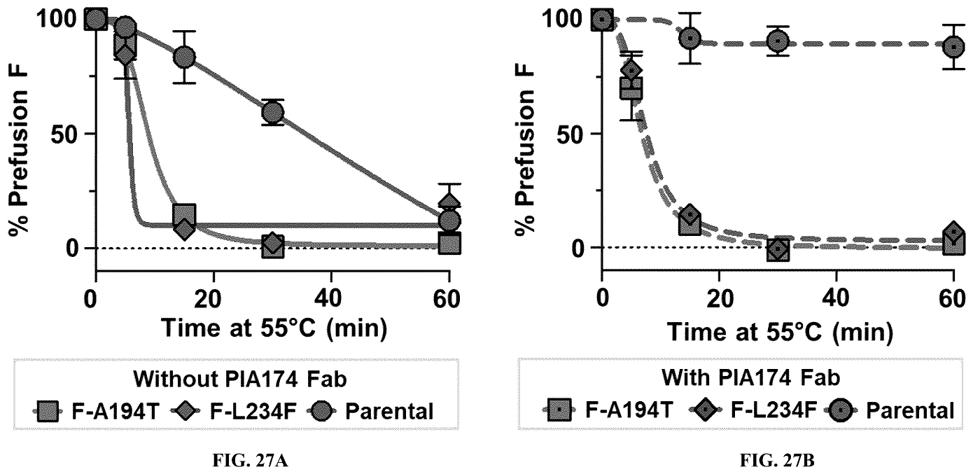

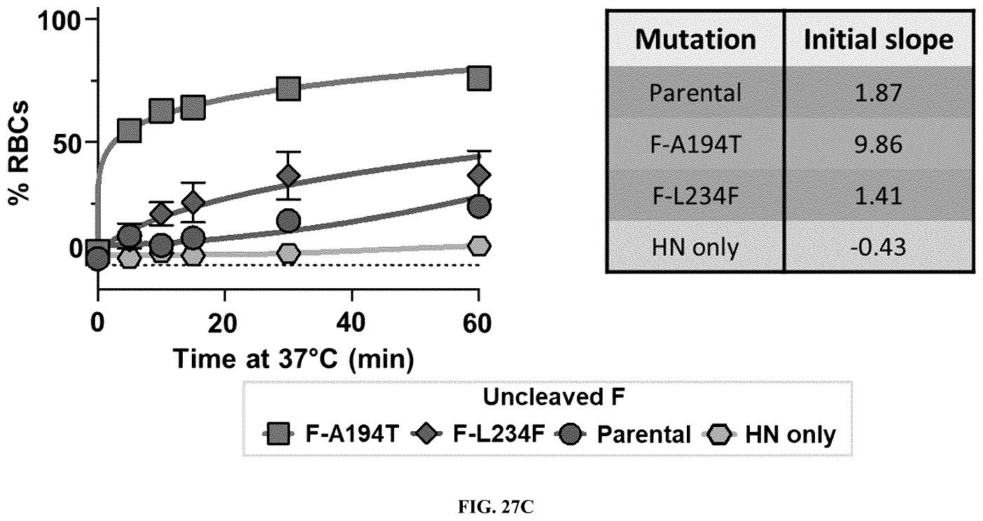

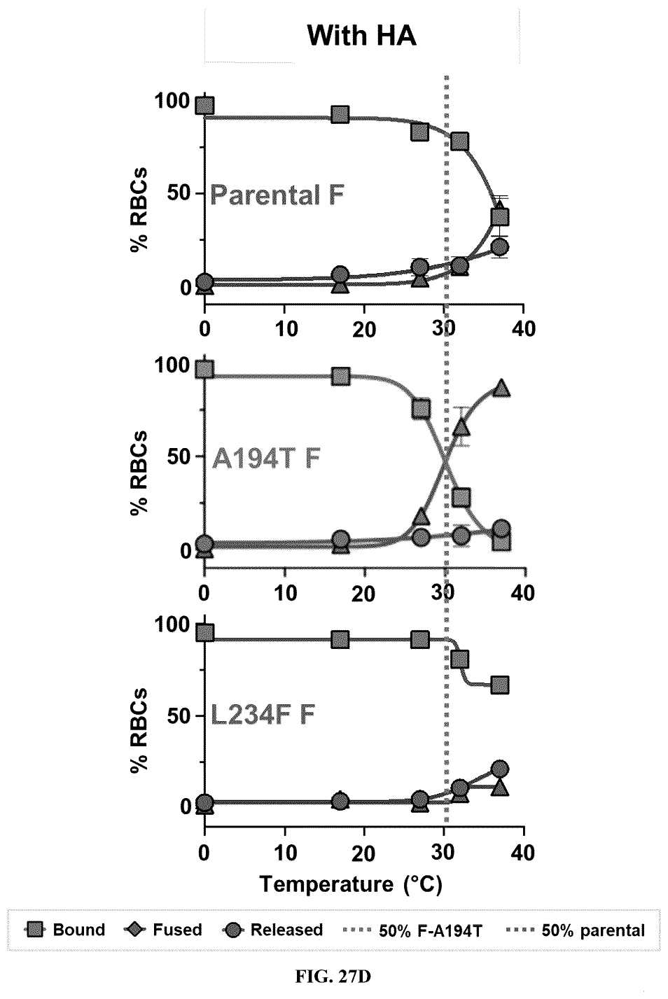

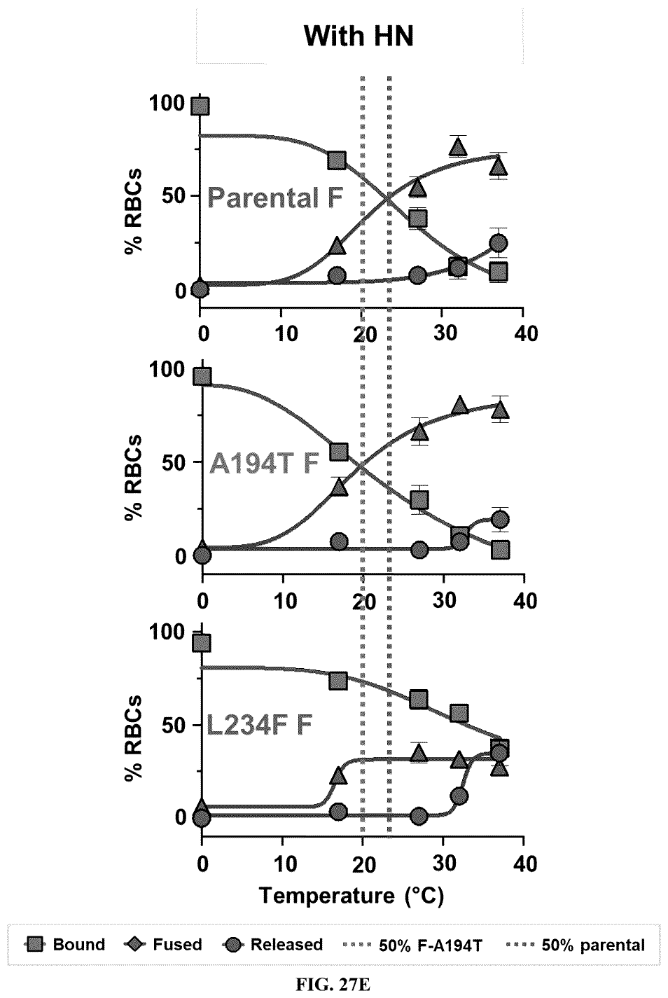

The PIA174 Fab neutralizes by stabilizing the pre-fusion state of F. Both escape mutations reverse this effect by conferring increased ‘triggerability’ of F from the pre-fusion state, i.e. facilitating F's exits from the prefusion state to initiate its conformational change. Cells expressing parental F, A194T F, or L234F F were incubated at 55° C. for different times and the % of F in pre-fusion state was detected using a conformation specific pre-fusion F antibody that binds a site distal to the apex of F (FIG. 27A). Compared to the parental F, both variant F proteins exited the pre-fusion state much more quickly, with the pre-fusion F signal gone within 15 min for the variants and 1 hour for parental F. While parental F is retained in the prefusion state by the presence of the PIA174 Fab in the face of increasing temperature (FIG. 27B), neither A194T F nor L234F F are stabilized by the antibody; they exit the pre-fusion state at the same temperature in the absence (FIG. 27A) or presence (FIG. 27B) of Fab.

To measure the rate of conformational change of the various F proteins, the ability of HPIV3 HRC-lipopeptides to capture the transient extended intermediate of F was exploited. Cells co-expressing uncleaved F bearing L234F, A194T, or no mutations and influenza HA or HN were allowed to bind to their sialic acid receptors on red blood cells (RBCs) at 4° C., then incubated at 37° C. to permit F activation (FIG. 27C). The use of uncleaved F proteins for this experiment allows specifically for measurement of the rate of conformational change of unfolding the pre-fusion F, without progress to F-insertion or fusion. The HRC-lipopeptides insert into the RBCs and capture extended F proteins, thereby tethering the RBCs to the F-expressing cells, without requiring membrane piercing or fusion mediated by F. Under these conditions, the F bearing L234F undergoes conformational change, extends and is captured by lipopeptide on the RBC, and does so at a somewhat higher rate than parental F—although not as rapidly as the F bearing A194T (FIG. 27C).

To evaluate the impact of the L234F F mutation on the ability of F to be activated by heat or by HN and to progress towards insertion in a target membrane and complete fusion, compared to the impact of the F apex mutation A194T, an assay was used that distinguishes between different states of F activation and quantitates all the states. The readout for F activation, insertion, and progress to fusion is fusion of RBC with F-expressing cells. Cells co-expressing cleaved F bearing L234F, A194T, or no mutations and influenza HA or HN were allowed to bind to their sialic acid receptors on RBCs at 4° C. and transferred to a range of temperatures to permit F activation. Influenza HA serves only to tether the target RBC to the F-expressing cells and does not actively trigger F; this condition measures activation of F by heat (FIG. 27D). At each temperature, we measured the percentage of target RBCs that were either released into the medium (blue circles), bound but had not fused, indicating that they were either bound by HN/HA (red square), or had undergone fusion (green triangle). The F bearing A194T is more readily triggered by heat compared to the parental F, in conditions where HA is used to tether but no HN is present. However, the F bearing L234F, despite more readily transitioning from the pre-fusion state as shown in FIG. 27A, does not progress to insertion in the target and to complete fusion; fusion mediated by this F co-expressed with HA is less than for the parental F; even at the highest temperatures used in the assay, 50% fusion was not attained for the L234F F. Thus, while both F proteins are more readily triggered to exit the pre-fusion state by heat, the L234F mutation appears to interfere with execution of fusion.

In the presence of co-expressed parental HN to activate F (FIG. 27E), A194T F is more activated and executes more fusion than parental F, but L234F F is much less readily activated than parental F. For parental F with HN, 50% fusion occurs at about 24° C., shown by the blue dotted line. A194T F with HN shows 50% fusion at about 20° C., shown by the red dotted line, indicating more efficient activation of F. L234F F shows less HN mediated fusion and fails to attain 50% fusion with HN even at 40° C., but nonetheless is not inactive, simply less (activatable) by their HN, indicative of the perturbation of a critical HN-F interaction site.

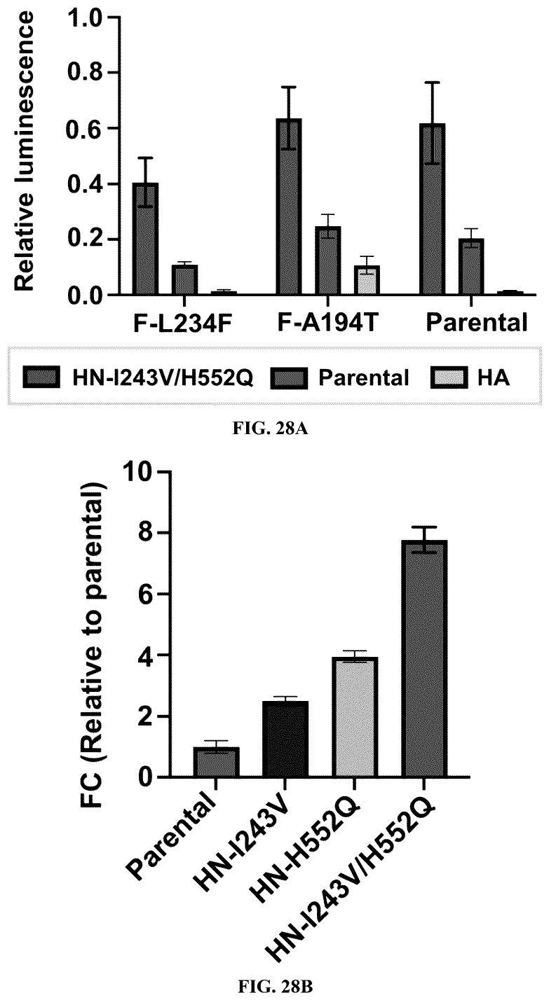

To understand how the L234F F can function in the context of an infectious virus, the effect of the HN present in the EV2 variant virus—HN bearing H552Q/I243V—on HN-promoted fusion mediated by the L234F F was examined. It has been previously shown that the H552Q mutation in HN confers enhanced fusion promotion. This residue comprises HN's sialic acid binding Site II, that has been shown to be critical for activating HPIV3 F; alteration of Site II affects fusion, infectivity, and viral fitness in vivo. The I243V mutation is in the globular head of HN at the dimer interface underneath H552Q (FIG. 26B), and its function has not been previously characterized. The effect of this mutated HN on fusion mediated by L234F F was determined using a cell-cell fusion assay where fusion of cells co-expressing HN/F or HA/F pairs with receptor bearing cells is quantitated by beta-galactosidase complementation (FIG. 28A). The H552Q/I243V is highly fusion-promoting and restores fusion by L234F F. The I243V mutation in HN has an enhanced fusion promotion effect and is additive in its fusion promotion effect with the H552Q dimer interface mutation (FIG. 28B). The HN with H552Q and I243V mutations has an enhanced fusion promotion effect on all three F proteins (FIG. 39A).

d. Cryo-ET Structure of Escape Variant HN-F Complexes at 10A Resolution Reveal Altered Relationships Between HN and F in the Complex

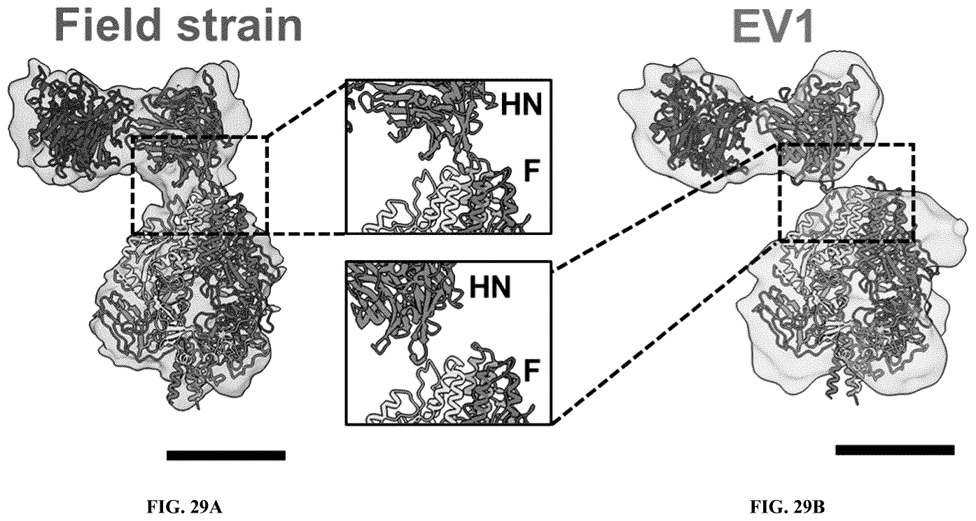

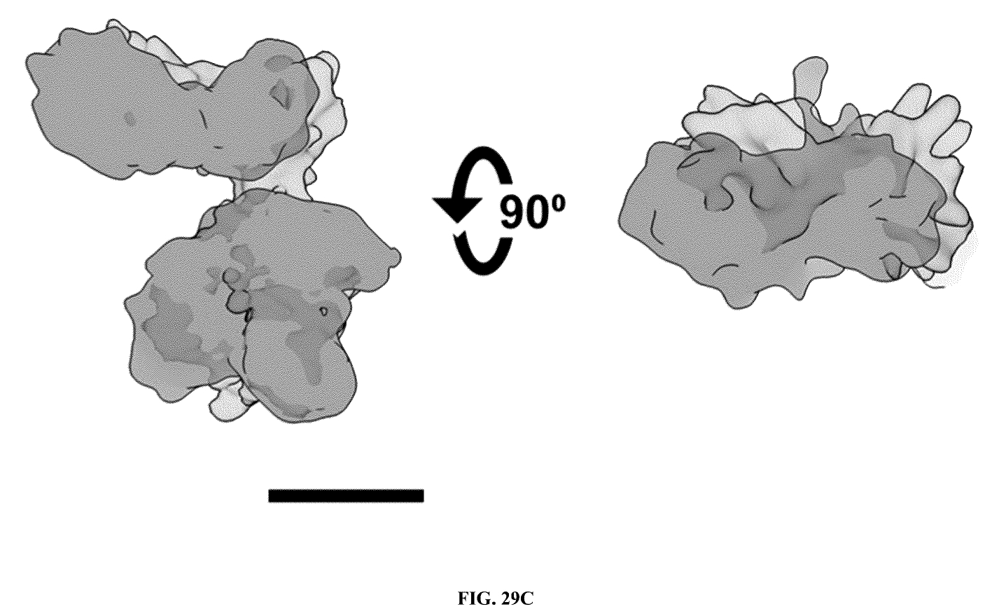

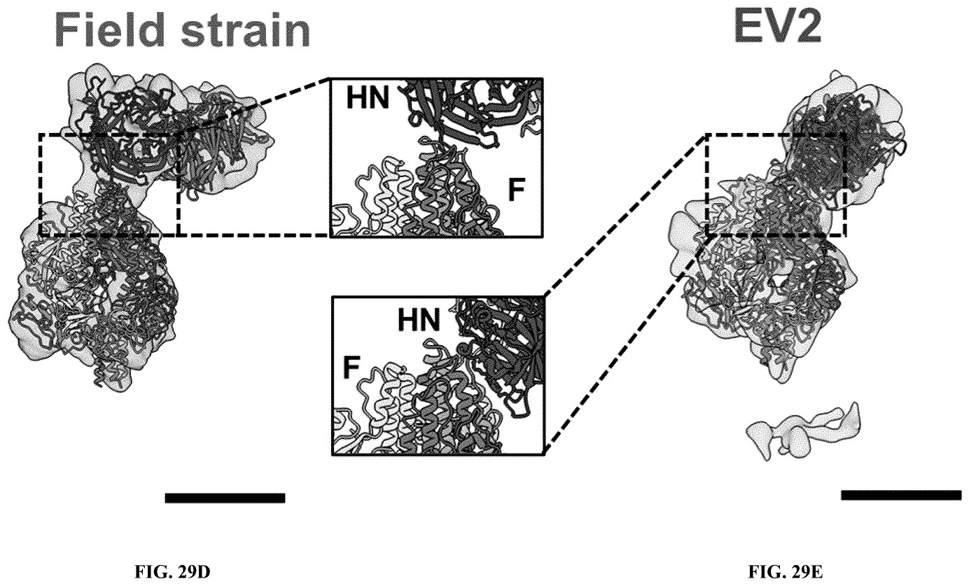

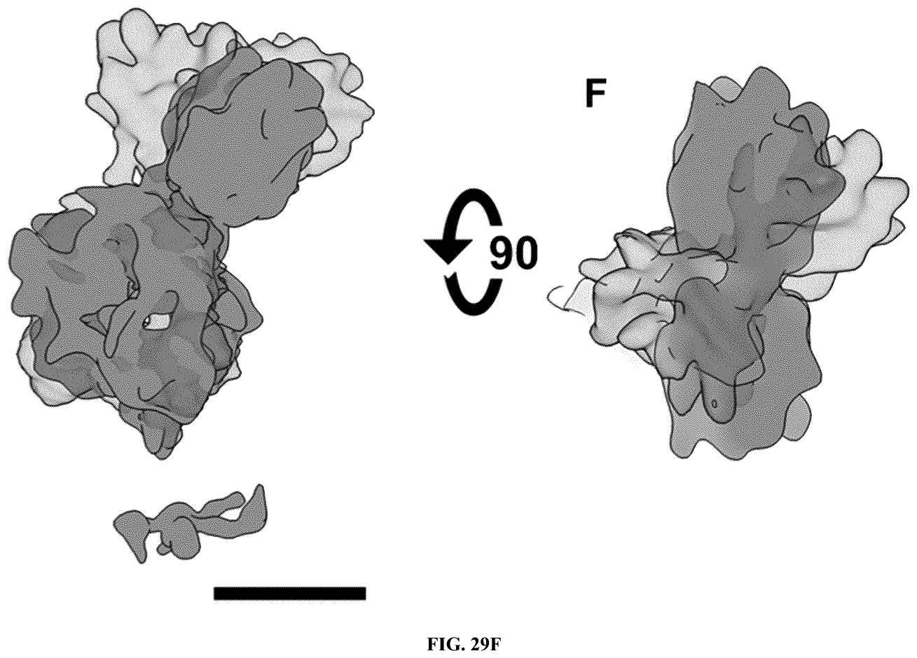

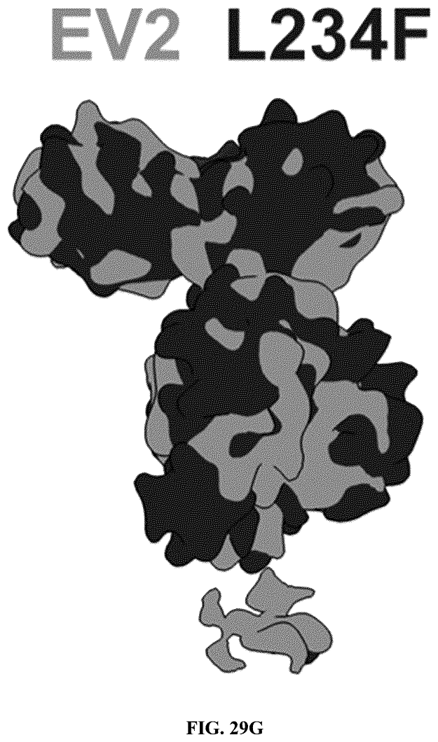

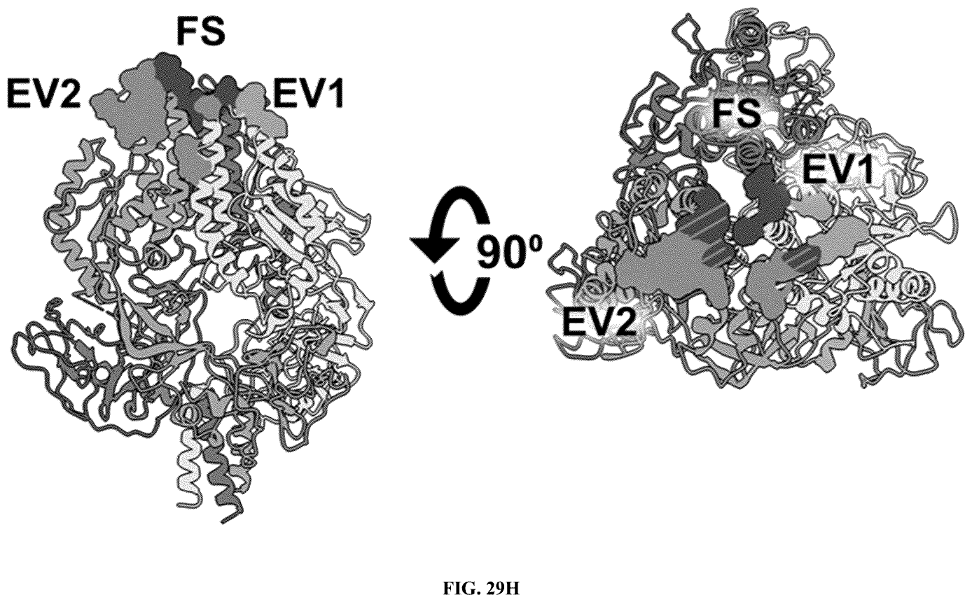

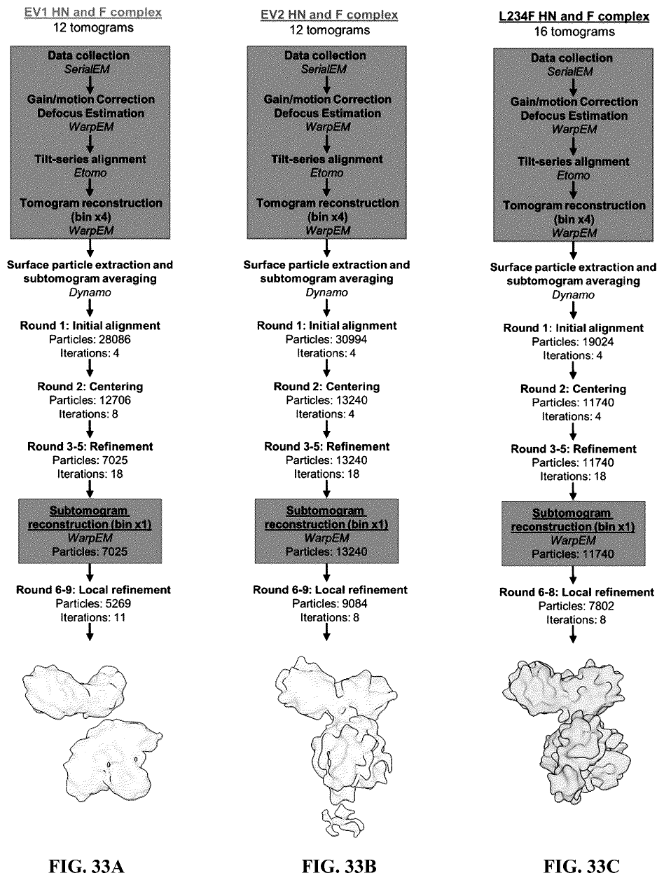

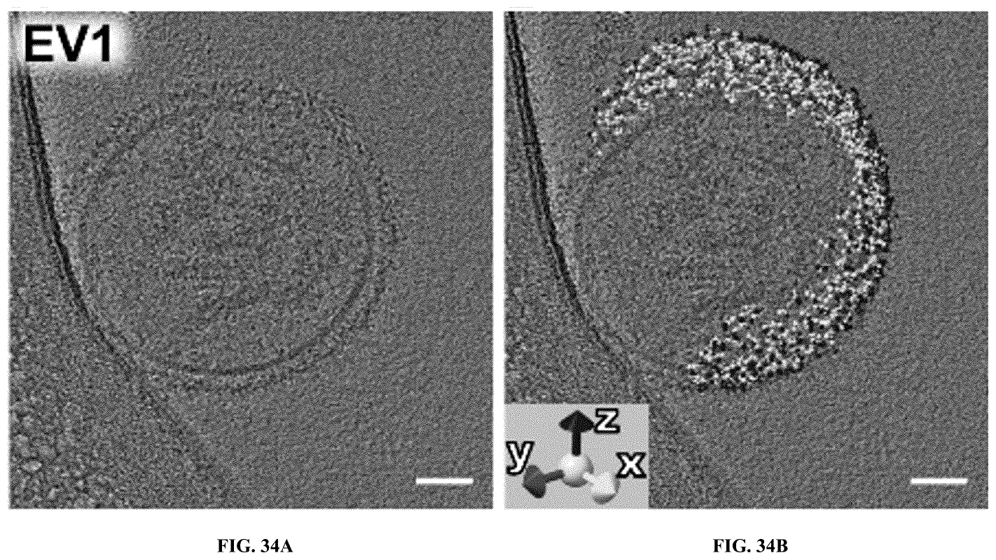

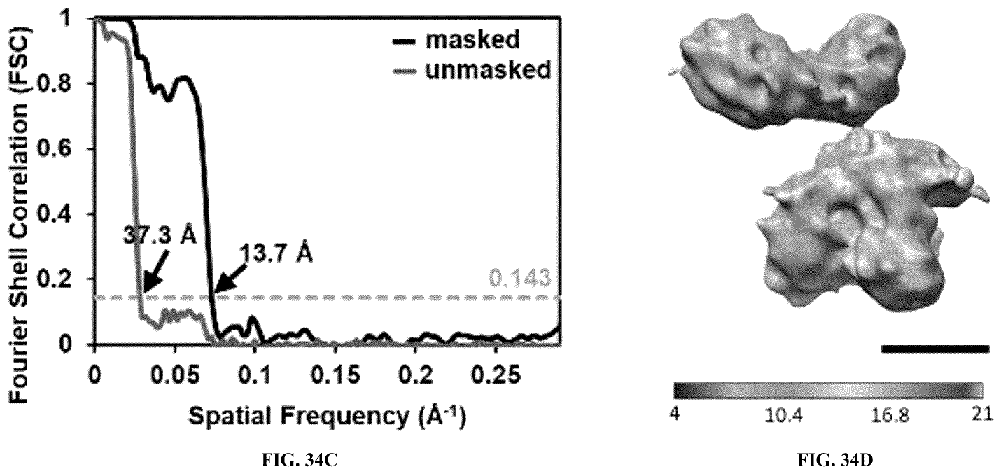

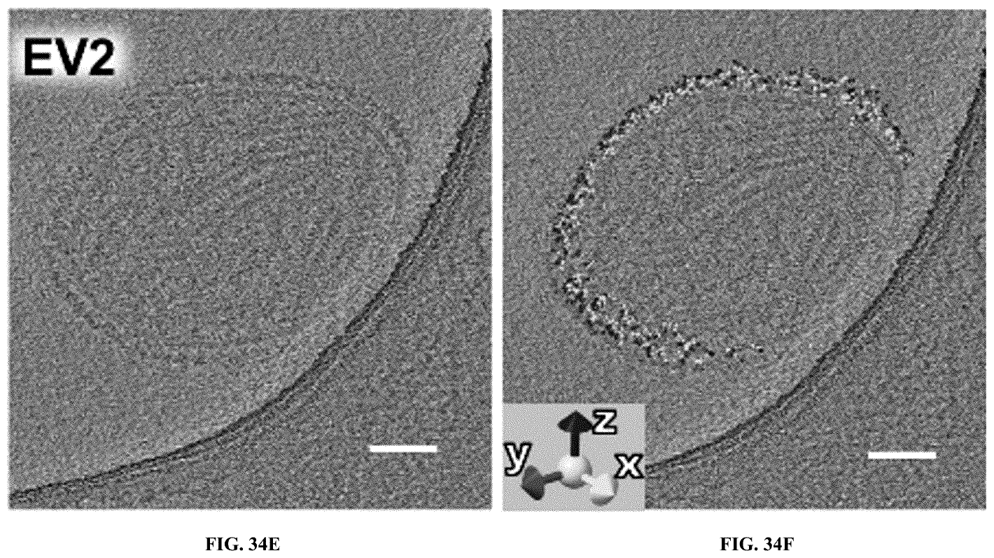

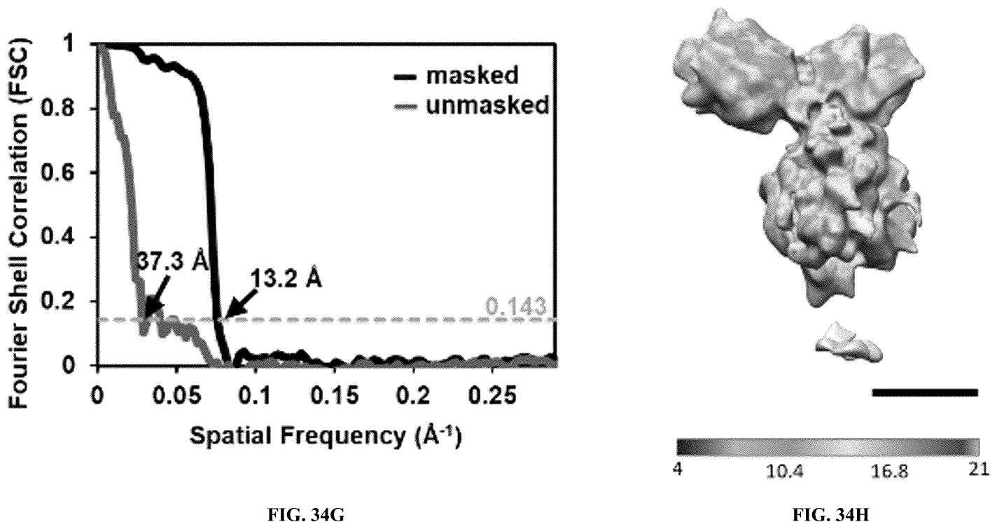

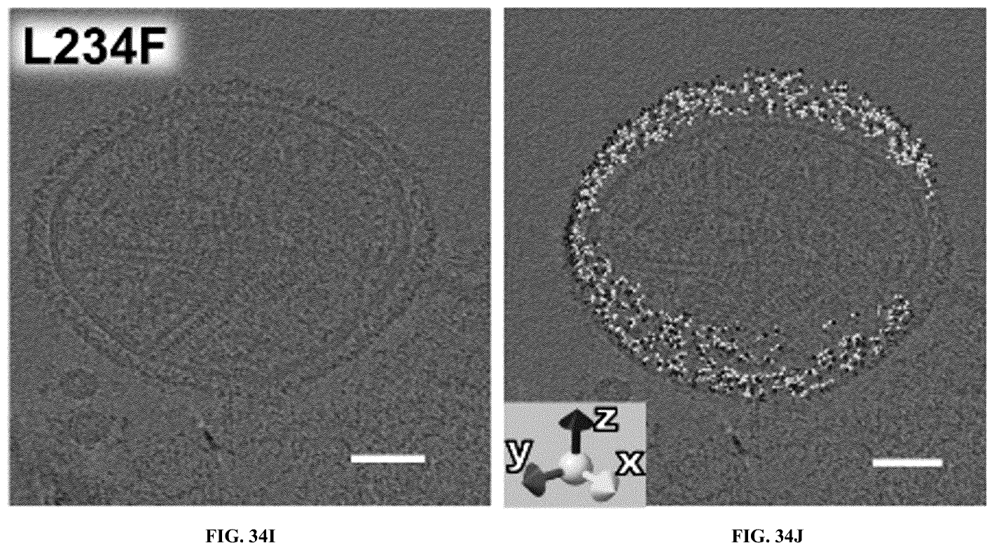

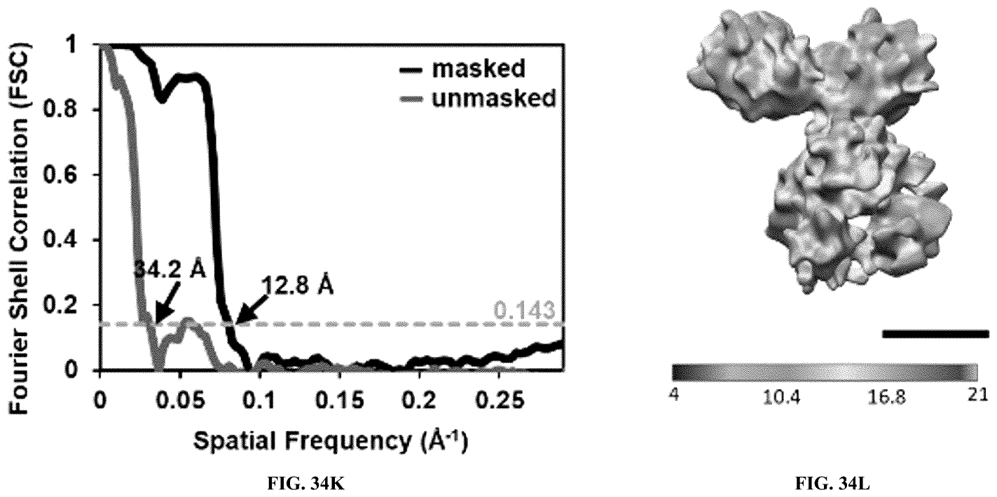

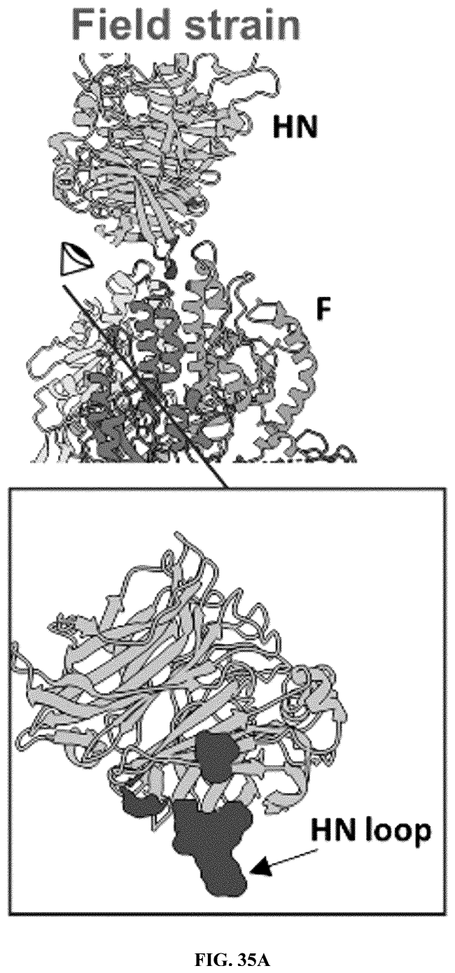

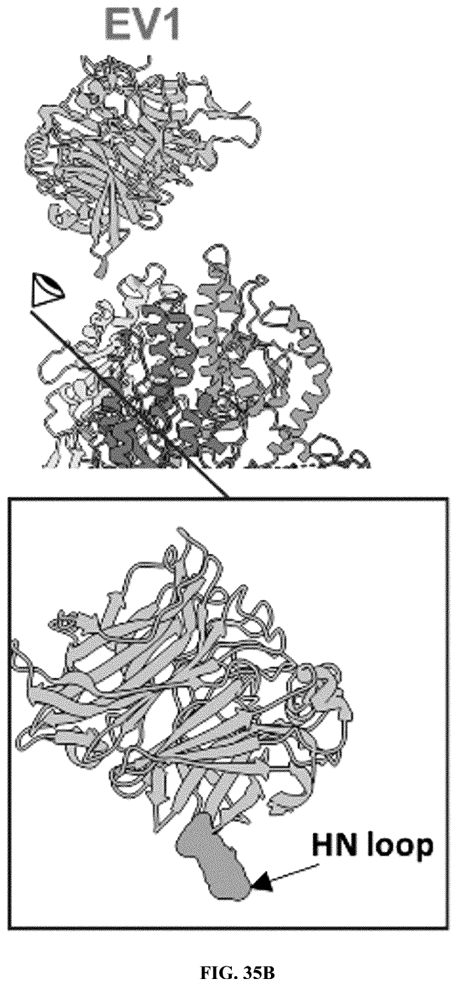

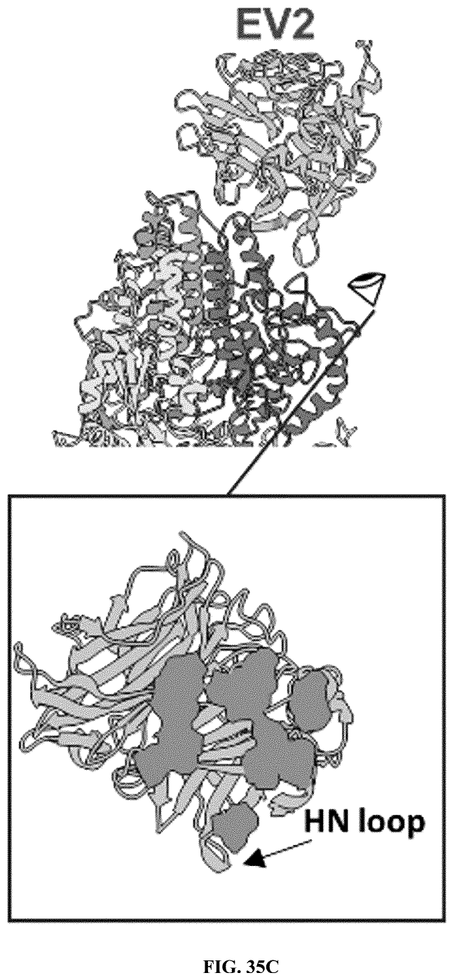

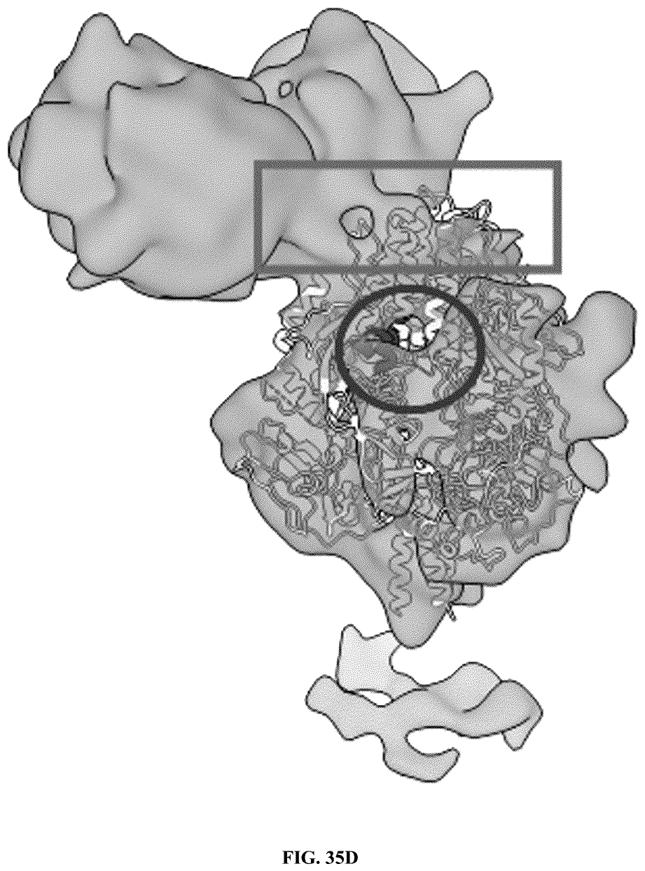

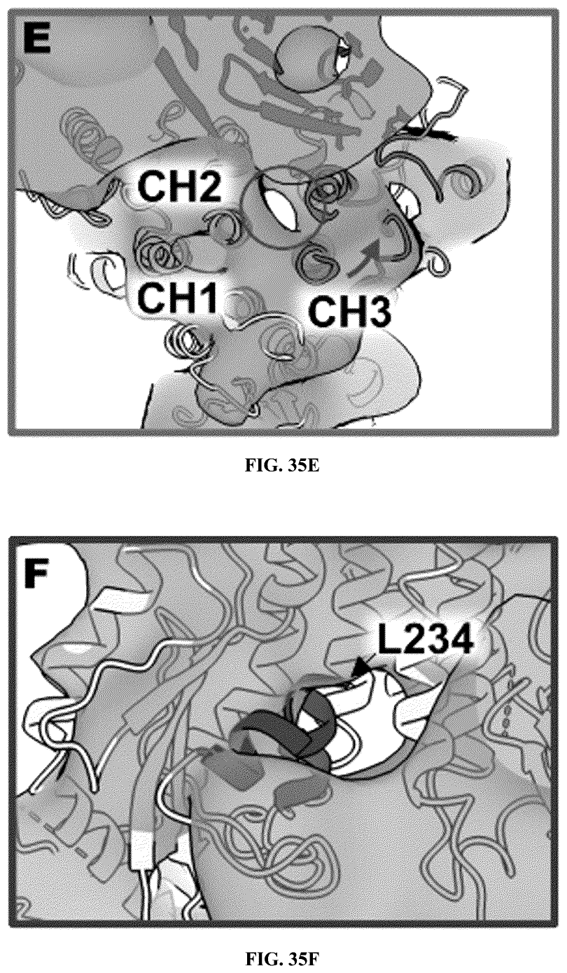

To examine the structural consequences of the Ab escape mutations on the HN/F complex on the viral surface, the cryo-ET structures of parental HPIV3, EV1, and EV2 HN/F complexes were compared. Antibody affinity capture to purify each viral sample directly on the grid without high speed centrifugation was used, using a procedure that avoids the disruption of surface glycoproteins associated with other purification methods. The previous processing pipeline for subtomogram averaging of viral surfaces to obtain medium resolution of the HN/F complex for each virus was used (FIGS. 33A-33C). The cryo-EM model of the soluble F (Protein Data Bank (PDB) ID: 6MJZ) was fit into the final density for each variant final subtomogram average (FIGS. 29A-29E). The models of individual HN protomer heads, derived from the crystal structure of the soluble portion of the HN dimer (PDB ID: 4MZA) were also fit into the final density of each subtomogram average (FIGS. 29A-29E). The model-to-map Fourier shell correlation (FSC) was between 11 and 13 Å for the final subtomogram averages (FIG. 34A-34L). At this level of resolution, it can be observed that remarkably, the HN-F interaction at the apex of F has been altered in the fusion complexes of both variants. The globular head of HN that—in the parental complex—sits directly atop the apex of F, capping it, is shifted off the F apices. The HN dimer heads extend 175 Å above the viral membrane surface as the heads rest above F in the parental HN/F complex (FIGS. 29A, 29D, and 35A), but only 160 Å above the viral membrane surface for both EV1 and EV2 (FIGS. 4B, 4E), where the HN head is not capping the F apex. The F apex is exposed in both viruses.

For both EV1 and EV2, the 386-392 loop in HN interacts with F differently to this interaction on the parental virus. The structure of the HN-F complex in EV-1 shows that, when viewed from the top, the HN protomer proximal to F is shifted forward off the apex compared to the parental HN/F complex, resulting in an interaction between HN and F over a smaller surface area, suggesting that it is a less stable interaction (FIG. 29B inset). The only interaction between the HN protomer and F is the 386-392 loop of HN that loosely interacts with the 56-64 loop in the F2 region of F, near the disulfide bond that connects F1 and F2 (FIGS. 29H-29I and 35B).

The EV-2 HN-F structure is different from that of the EV-1 complex, in that, when viewed from the top, the HN protomer proximal to F rotates and shifts laterally off the apex making extensive interactions with the F-HRC region adjacent to the central helical core (FIG. 29E inset). The HN 386-392 loop is no longer in contact with the F protein (FIG. 35C) but the buried surface area between the HN and F increases from 490 Å in the parental complex to 608 Å in the EV2 complex (FIGS. 29H-29I). The structure of the fusion complex for a recombinant virus with F bearing only the L234F change was solved to examine whether the HN mutations in EV2 contribute to the displacement of HN from the apex of F, or whether this displacement is attributable to the L234F mutation in F alone. This complex bearing that differed from the parental virus only in the L234F mutation in F showed the same displacement of the HN head from the F apex as EV2 (FIG. 29G).

The lack of density at the apex for both viruses shows that the structure at the apex trimer interface is now looser in both EV1 and EV2 (FIGS. 35D-35F). For EV2, in distinction to parental (and to EV1), it is noted that the domain adjacent to the central helix of the F trimer is also more flexible in structure, as evidenced by the lack of density at the domain adjacent to the central helix.

e. Escape Mutations Exact a Fitness Cost for the Virus in Human Airway

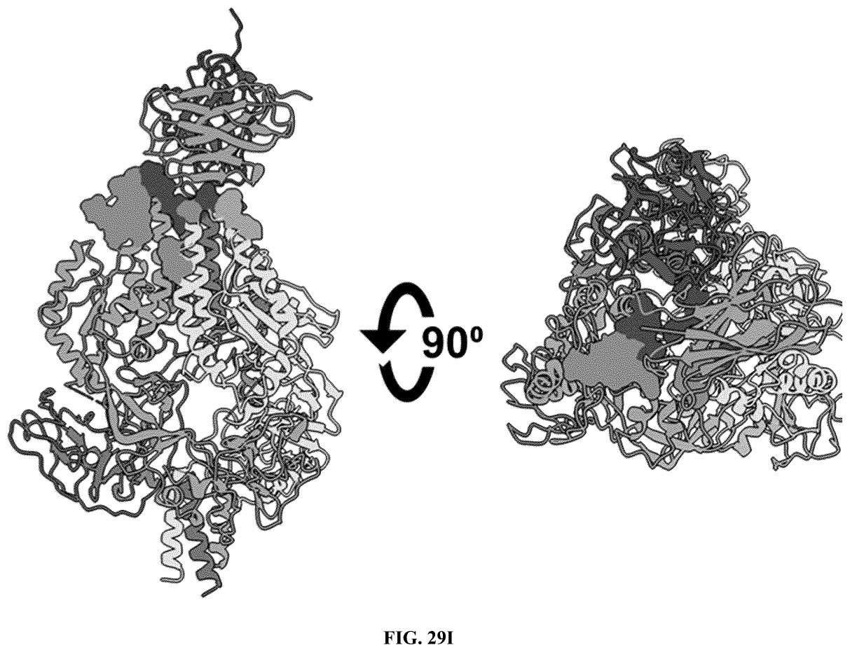

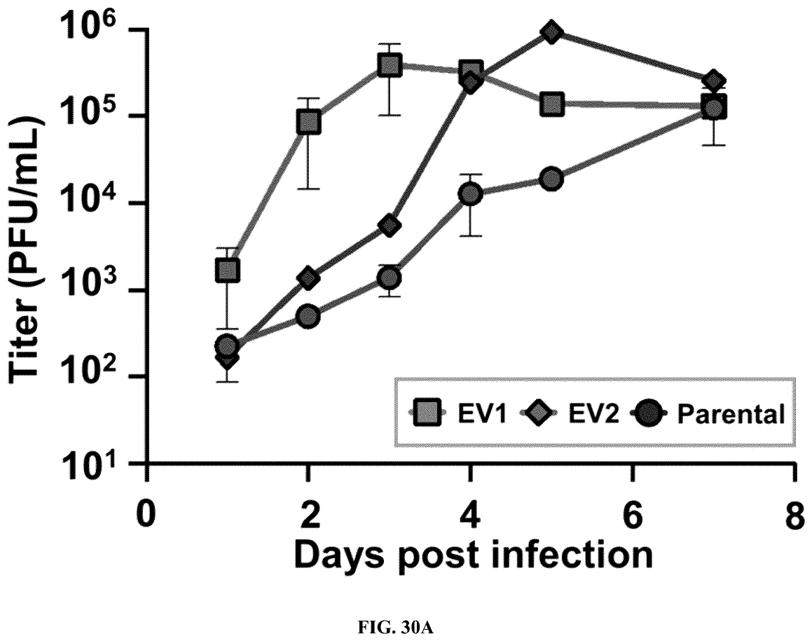

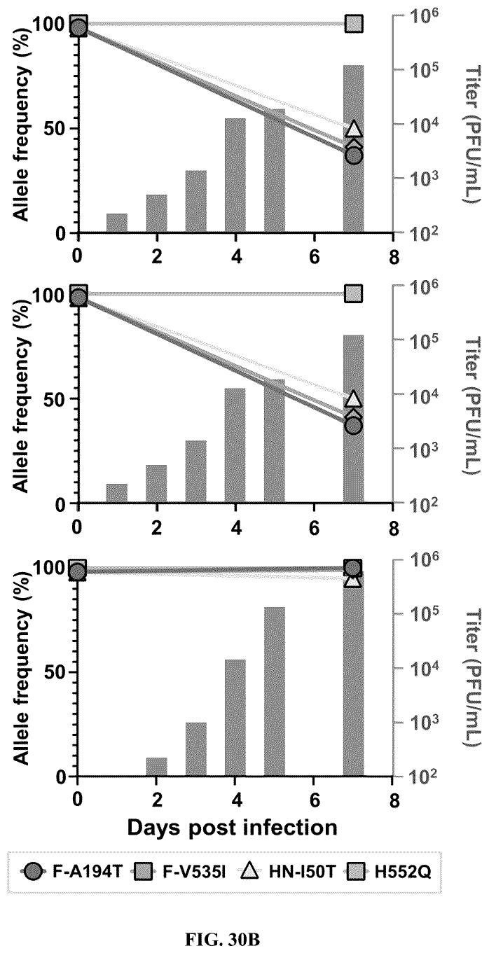

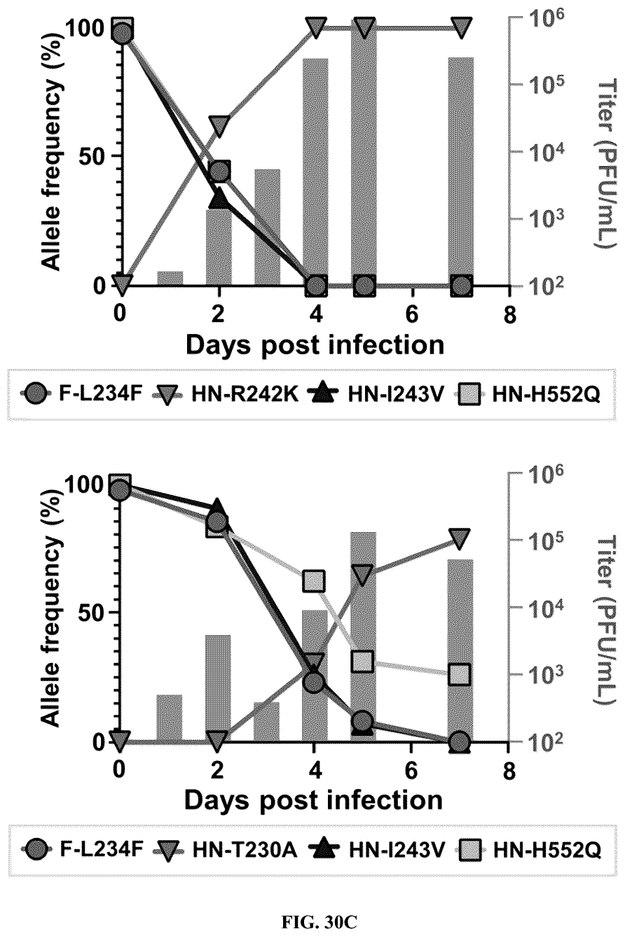

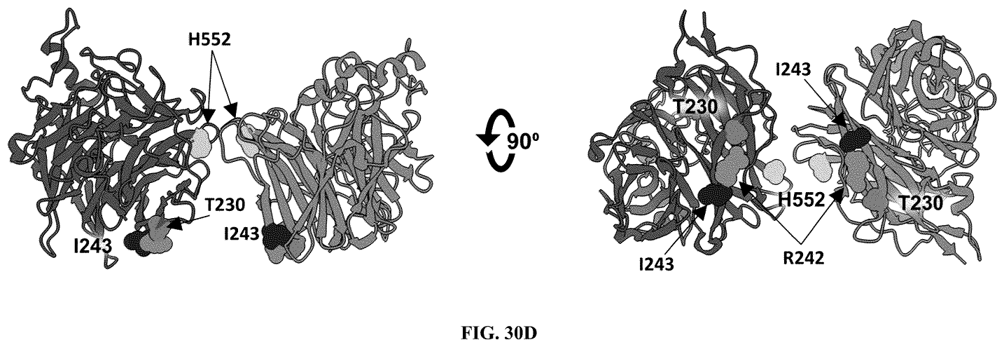

To assess the fitness consequences of the mutations in the viruses that emerged during selection for escape from PAI174, it was determined the consequences of the evolution to infection in human airway epithelia (HAE) (FIGS. 30A-30D). It has been shown that HAE cultures represent an authentic model of the human lung, reflecting the cell environment and selective pressure of the natural tissue. These airway cultures have been well-validated for evaluating features of infection and HPIV3 fitness. The growth and evolution of the variants was compared to the parental strain. HAE cultures at an air-liquid interface were infected with EV1 or EV2 viruses or the parental virus and viral titer measured daily for 7 days (FIG. 30A). Compared with the parental virus, EV1 showed significantly reduced production of infectious viral particles (red line) which only reached the parental level (blue line) by day 7. For EV2 (green line) there was an initial delay in production of infectious virus but beginning at day 3 the growth curve dramatically changed and by day 4 overtook the titer of the parental virus.

Alterations in HN or F that confer a disadvantage in the human airway will lead to evolution. It has been shown that the HN-F fusion complexes of HPIV3 field strains from humans are not under selective pressure to evolve during growth in HAE. By growing the variant viruses in HAE, it is determined whether these mutations place the virus at a disadvantage and under selective pressure to evolve in the HAE. Remarkably, deep sequencing of the day 7 viruses revealed that while EV1 retained its escape mutations—which resulted in slowed growth that caught up—EV2 had entirely reverted to the parental sequence under the selection pressure of growth in human airway (FIGS. 30B and 30C). This rapid response to airway conditions revealed the severe fitness cost of the L234F change in the background of EV2. EV1, despite having a fitness cost, is not significantly impaired in essential functions and did not exhibit a disadvantage significant enough to spur evolution. FIG. 30C shows the daily accumulation of changes in EV2 until by day 7 the reversion is complete. In each well, a single mutation arose in HN (R242K for well 1 or T230A for well 2) and rose in frequency over the days, as the EV2 mutations decreased to complete loss. In contrast, FIG. 30B shows that EV1 retained all its mutations including the F-A194T apex mutation. The I243V mutation (dark blue spheres) that arose in EV2 during viral evolution is adjacent to both R242K (brown spheres) and T230A (cyan spheres) (FIG. 30D) and is located in the dimer interface distal from the H552 residue (FIG. 30D, yellow spheres).

### Example 4. The Receptor Binding Protein (HN) Prior to Receptor Engagement: Maintaining the Complex in a Stable Pre-Fusion State

It has been previously shown biochemically that HN and F associate prior to receptor binding, and that prior to receptor engagement, HN stabilizes F to prevent F's premature activation. The interface between HN's globular heads in the HN dimer modulates HN-F interaction and fusion. Now, to understand the HN-F complex prior to receptor engagement on authentic viral surfaces, virions directly from infected cell supernatant without any purification steps were isolated, attached them directly to cryo-EM grids, and showed stable interaction be-tween HN and F. Sub-nanometer resolution of the HN-F fusion complex in the prefusion state on the surface of an authentic clinical virus with cryo-ET was recently attained, sufficient to answer mechanistic questions. An HN loop reaches downward to insert in a pocket in the apex of F, suggesting how the fusion complex is maintained in a ready but quiescent state until activation. The HN globular heads are rotated with respect to each other; one downward to contact F, the other upward to grapple cellular receptors, intimating how distinct steps prior to F activation may unfold. This is the first example of such resolution for a paramyxovirus fusion complex, with authentically situated envelope proteins directly on a virus. There are partial structures of soluble HN and soluble F alone, but until now, no structure of the HN-F complex. Examining intact viral surface complexes is crucial—the structures of the complexes shown here differ from their soluble counterparts. This (& higher) resolution of complexes on authentic viral surfaces permit analysis of HN-F interaction and fuel hypotheses about function to be tested using a panel of functional assays. These structures are key to answering outstanding, important, questions about how the prefusion HN-F complex functions and maintains readiness for entry at the right time.

a. The Cryo-ET Structure of the Pre-Receptor Engaged HN-F Complex on Clinical Virions to Address the Mechanisms for Stabilization of Pre-Fusion F in the Complex

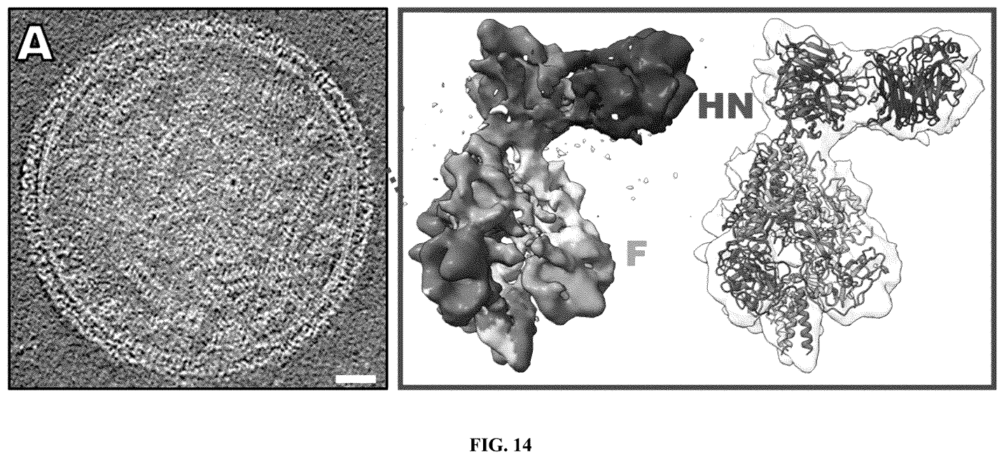

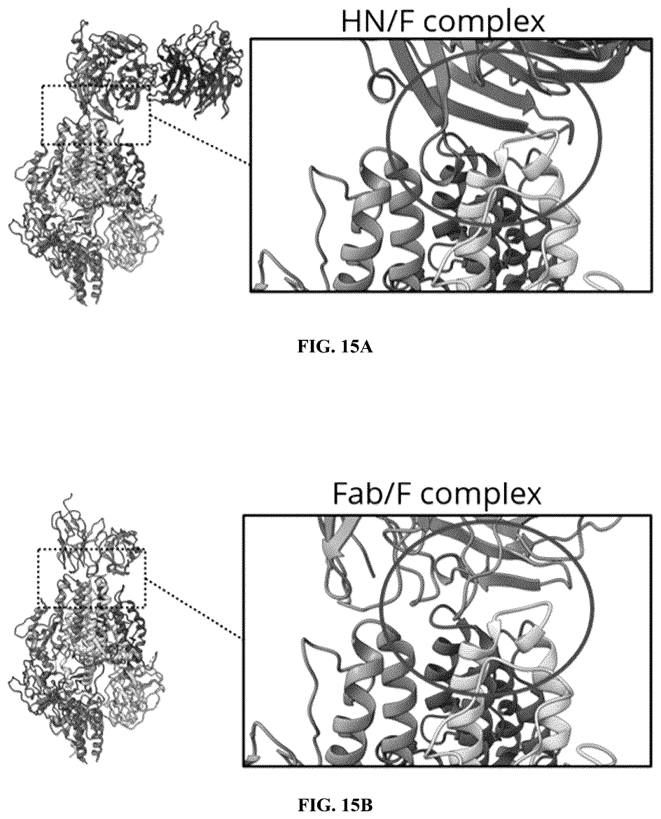

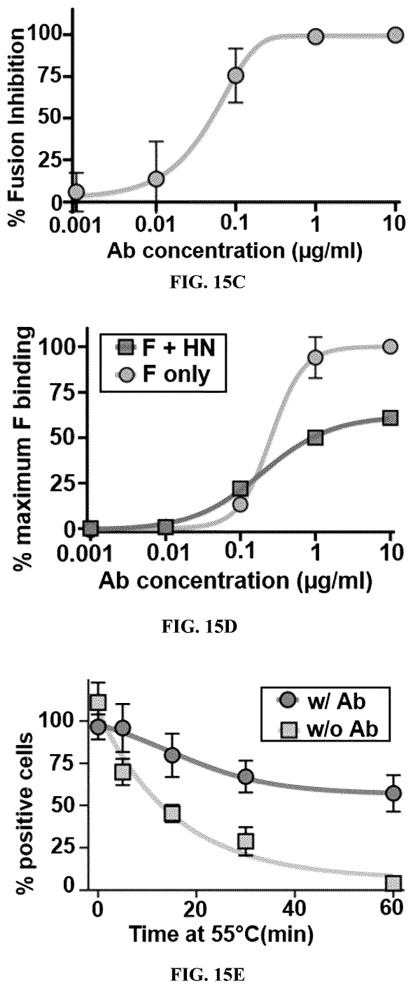

To achieve the desired resolution of the surface glycoproteins prior to receptor engagement recombinant viruses bearing the HNs and Fs from clinical strains were used, which represent true “wild-type” viruses that are infectious for humans, with “CI-1” as the backbone. Imagining viruses varying temperature and time (“Condition 0”) to optimize experimental conditions and incubation times and optimally enrich for imaging by cryo-ET, proceeding from conditions used for the data in FIGS. 14A-14C and 15A-15E. Existing structures for HN and F—and published crystal structures of HPIV3 HN, a lab-adapted strain and a lung adapted variant (HN H552Q/Q559R), with and without ligand—are used to interpret and compare with the cryo-ET density maps.

FIGS. 14A-14C shows cryo-ET subtomogram averaging of HN-F interaction before receptor engagement. The HN dimer heads extend above the viral membrane surface, atop F. While portions of the F stalk are visible, we do not observe the long alpha-helical HN stalk, likely due to its flexibility. At the final resolution, the head of one of the two HN monomers interacts with the apex of F, while the other is positioned distally.

### Example 5. Relationship Between HN and F in the Pre-Fusion Complex

The globular head of the HN monomer that is directly above F contains a loop composed of residues 386-392 that extends downwards to engage with residues 180-190 at the apex of the head of the F trimer (FIG. 15A). This 386-392 HN loop nestles in the space formed between the monomers of the F trimer, occupying a hydrophobic pocket at the apex of F. The interaction creates a “thumb-finger” motif in HN facing the apex of F, where the “thumb” is the 386-392 HN loop and the “finger” is a beta sheet and second loop comprising HN residues 294-300 (FIG. 15A). Within the HN loop, residues K386, L388, S390, and N391 appear to make contact with residues on F including A194. It is hypothesized that this interaction helps maintain stability of the pre-fusion HN-F complex before receptor engagement.

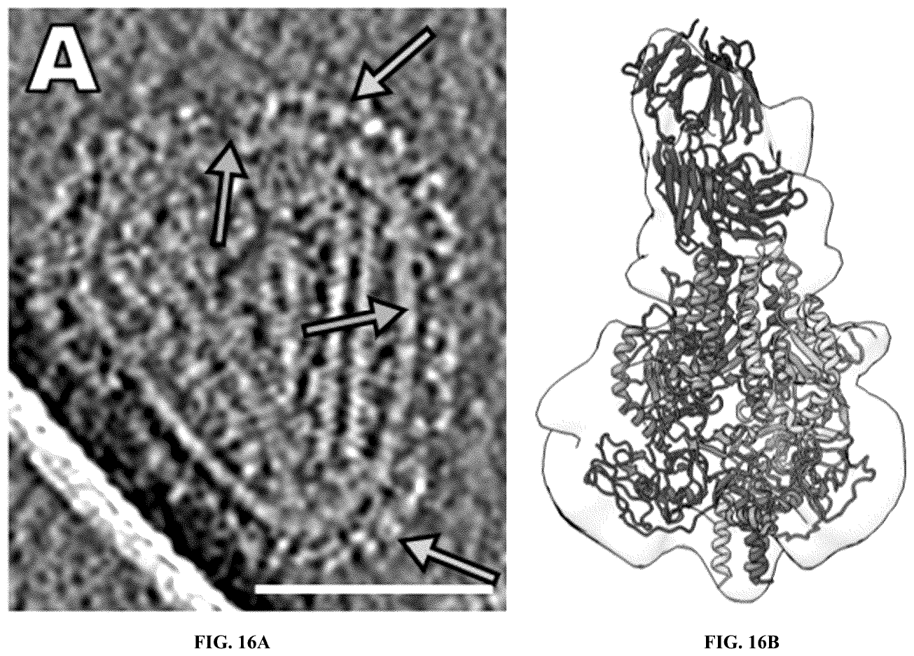

Remarkably, an anti-F neutralizing antibody that inhibits infection (PIA174) binds to the apex of F at antigenic site Ø, at the same site as the HN 386-392 loop binds to F in the HN-F complex (FIGS. 15A, 15B). The structure of PIA174 Fab complexed with the stabilized soluble region of prefusion F was solved by cryo EM60 (FIG. 15B). Surprisingly the Fab contains a “thumb-finger” motif facing the apex site of F similar to the HN loop, evident in the side-by-side comparison of the Fab atomic surface with the cryo-ET model of HN and F (FIGS. 15A and 15B, red circles). This overlap leads to our hypothesis that both the antibody's neutralizing property and HN's stabilizing property derive from interaction at that site of F. The PIA174 Ab blocks fusion in the presence of HN (FIG. 15C). In the presence of HN there is less PIA174 Ab binding to F (FIG. 15D), suggesting that HN and Ab bind to the same site on F. The Ab PIA174 maintains the stability of the pre-fusion F in the presence of heat (FIG. 15E), suggesting that the Ab stabilizes the pre-fusion F, similar to the stabilization of F by HN that has been previously shown. In our cryo-ET subtomogram averages at 4° C. the presence of the PIA174 Ab is seen to perturb the HN-F complex (FIG. 16A). Based on these data, it is hypothesized that HN's F-interacting loop stabilizes F like the PIA174 Fab (FIG. 16B), and that mutations in this loop will affect HN's stabilization of F. These predictions will be tested.

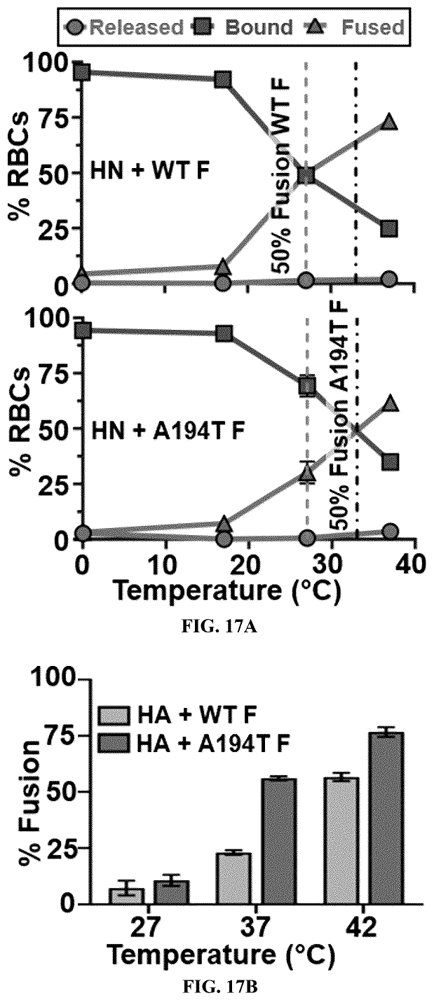

To evaluate the function of the HN loop we will use mutations derived from both structural and functional information. In a viral evolution experiment virus was grown in the selective pressure of PIA174 Ab. Two mutations arose in escape variants; F-A194T at the F apex, and HN-H552Q at the HN dimer interface with enhanced F activation properties. (Remarkably, both have emerged during persistent infection in humans.) The F A194T mutation decreases the ability of the PIA174 antibody to bind F. The F A194T is more readily triggered by heat compared to wt F, but less readily activated in the presence of HN (FIGS. 17A and 17B), suggesting that the HN-F interaction has been perturbed. The HN-H552Q escape variant bears an HN dimer inter-face mutation that we know to increase receptor avidity, increase HN-F interaction, and increase activation of F. The preliminary findings support the notion that this HN-F interaction site represents an important and unexplored site of interaction between HN and F.

The effects of 4 specific sets of mutations at the site of HN-F interaction (in the HN loop comprising residues 386-392, or at the F apex) will be assessed for their impact on HN-F complex stabilization and function: (i) HN G387S, a mutation in the loop that emerged in individuals persistently infected with HPIV3 and modified the fusion phenotype of those viruses and during an HPIV3 outbreak during the COVID-19 pandemic; (ii) HNs with charged residue substitutions at residues 388, 389, and 390; (iii) HN where the loop is substituted with the corresponding loop from the PIA174 Fab; (iv) F A194T, the mutation in the F apex that emerging during evolution in culture to escape neutralization by Ab PIA174 and in individuals persistently infected with HPIV3.

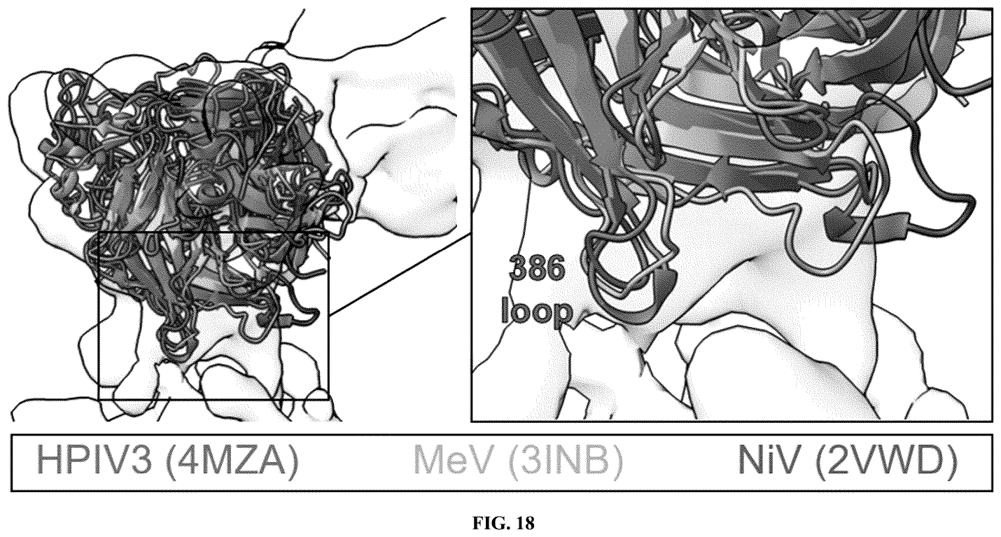

Revealing this site of HN-F interaction will offer a new target for antivirals—interrupting the pocket in F, the loop on HN, or the interaction at that site will inactivate infectivity. Most exciting (FIG. 18), this HN loop structure that interacts with the apex of F is highly conserved across receptor binding proteins of paramyxoviruses suggesting a general mechanism for stabilization of the pre-fusion complex. It has been shown that the heads of several paramyxovirus receptor binding proteins can be swapped without affecting their F-activating properties or the function of the fusion complex. For example, Nipah virus F works when paired with a receptor binding protein composed of Nipah virus G stalk+NDV HN head, measles virus F works in a complex with a receptor binding protein composed of measles H stalk+either an NDV or an HPIV3 HN head, and HPIV3 F can complex and mediate fusion when paired with an HPIV3 HN stalk and NDV HN globular head, indicating a shared mechanism operating across multiple paramyxovirus receptor binding protein heads. Despite a lack of sequence homology between these heads, the HN loop—F pocket interface is highly conserved (FIG. 18). This HN-F loop-pocket interface could offer a new broad paramyxovirus antiviral target site.

The effect of the 4 specific sets of mutations described above on the stability of the pre-receptor engaged complex will be assessed by biochemical and functional assays. It is aimed to obtain high-resolution structures (5-8 Å) of the fusion complexes on mutant engineered viruses bearing the complexes that have the most significant functional effects, to correlate structure with biology and learn what the clinical virus requires to maintain its fusion complex stable until receptor engagement. Structure-function correlation of this important area of contact between HN and F will be a significant focus.

To improve structural resolution of the HN-F complex to 5-8 Å to resolve secondary structures (at 8 Å, alpha helices; at 5 Å, beta sheets), the subtomogram averaging pipeline optimized for FIGS. 14A-14C and 15A-15E will be used. Using these new methods, and in-house generated scripts, the process of subtomogram averaging was semi-automated decreasing the time from tilt-series collection to final subtomogram averages of the HN/F complex at sub-nanometer resolution. To improve to 5-8 Å resolution, the tomogram collection and our sub-tomogram particle size will be increased 10-fold. For the 9.1 Å resolution HN-F interaction density map, 12,000 particles were incorporated; increasing the particle size will allow us to classify different conformations of the HN/F complex and obtain a more homogeneous subset of particles leading to higher resolution. With this high resolution models will be built into our density maps to identify residue contacts between HN and F and also resolve the trans-membrane domains (important for fusogenic activity and F stability).

The contacts between static HN and F will be examined, and the effects of alterations in those contacts observed, in order to dissect mechanism. HN-F interaction during receptor engagement will also be characterized. Obtaining the structure of the HN-F complex, not just HN and F individually, has been a goal of paramyxovirologists for many years; this is a truly exciting accomplishment—and obtaining higher resolution will provide information that the field has been eagerly awaiting.

b. Determine the Structural Consequences of Mutations that Alter HN's Receptor Binding and HN-F Interaction

Viruses bearing wt and mutated HNs and Fs—some that we have in hand and others that being generated based on predictions from structural data will be imaged. Comparisons between variants whose altered function is well understood will help validate our correlation of structure to function.

The working hypotheses are: (i) interaction at the dimer interface between HN globular heads affects the complex and state of F; (ii) The interacting loop on the underside of HN's globular head (FIGS. 14A-14C and 15A-15E) binds with the apex of F and regulates viral infectivity; (iii) shifts between the globular heads are key for activation of F in the complex. The HN globular heads' mobility influences the actions of the HN stalk and impacts interaction with F. To address these hypotheses, biochemical and functional assays will be performed. With several well-characterized mutant HNs and one mutant F that is slower to activate (Table 1), the structure-function correlates of the pre-receptor-bound complex on the viral surface will be dissected. Most of the mutant viruses in Table 1 as well as CI from clinical samples have been obtained and understood the molecular and functional detail, including the role of specific domains of HN and F in growth in the host. These viruses provide the comparisons to guide the structural analysis.

TABLE 1
Selected HPIV3 HN or F variants used in the proposed studies.

| | Fusion promotion (T° | | |
|---|---|---|---|
| HN mutation | of activation by HN)^{A} | | Property of variant |
| Reference strain | 27° | C. | |
| H552Q^{B} | 13° | C. | Hype-triggering & avid (site II); PIA174 F- |
| | | | neutralizing Ab escape mutation |
| T193A/H552Q | 15° | C. | Highest avidity (sites I & II) |
| S554C | 15° | C. | Disulfide stabilized HN dimer (single) |
| N248C/S233C | ND | | Doubly Disulfide stabilized HN dimer |
| S554C/N248C/S233C | ND | | Triply disulfide stabilized HN dimer |
| H552Q/Q559R^{C} | 32.5° | C. | Dimer interface crystal structure |

| | | |
|---|---|---|
| G387S | ND | Mutation in HN loop (emerges during human |
| | | persistent infection) |
| PIA174 Ab loop | ND | Engineered HN with loop domain replaced |
| | | by Ab loop |

F mutation

| G396D | >37 | | Impaired activation (F) |
|---|---|---|---|
| A194T | 33° | C. | PIA174 F-neutr. Ab escape mutation |

^{A}Temperature at which fusion is 50%. Lower temp = higher F-triggering efficiency.
^{B}HN H552Q → ehanced fusion promotion.
^{C}HN H552Q/Q559R is present in a virus that evolved to grow in HAE. Compensatory mutation in HN (Q559R) reduces fusion and restores viral grovth in lung.

c. Relationship Between HN Globular Heads in the Complex

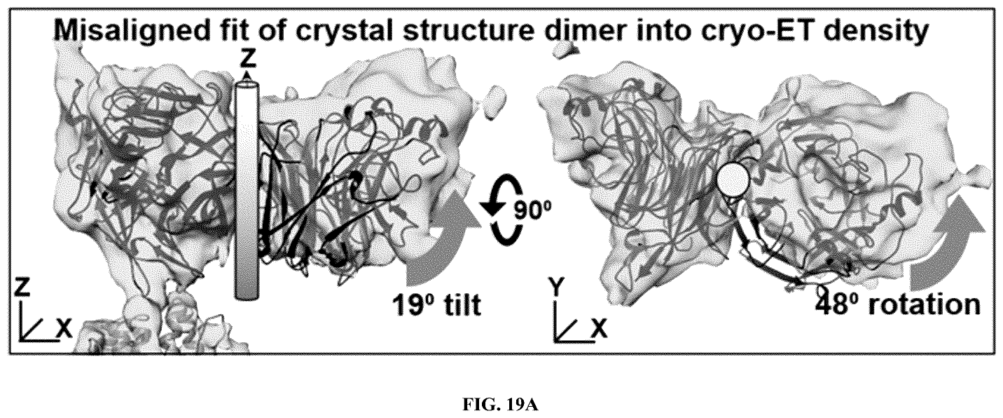

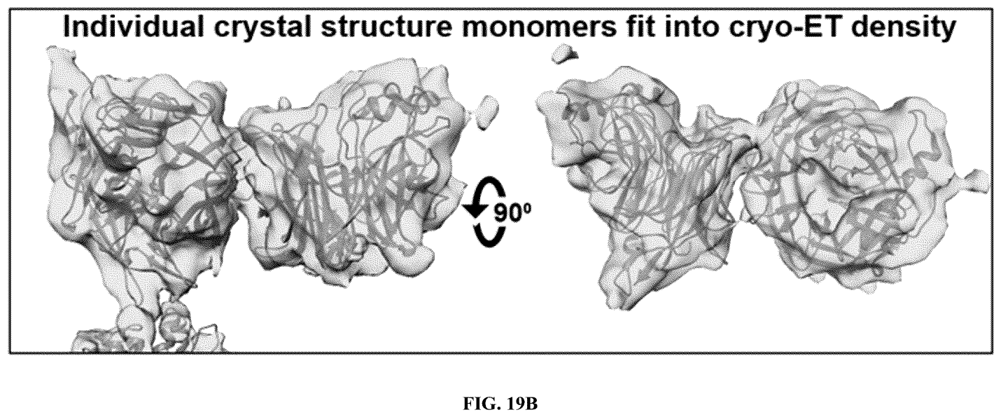

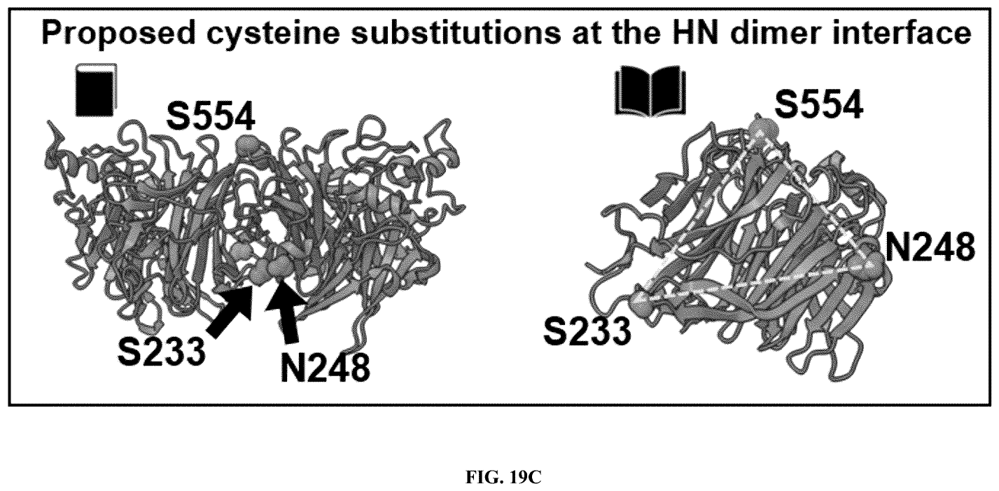

In the cryo-ET structure of the HN-F complex in an authentic state on the virus surface (9.1 Å resolution), the globular head of one HN monomer—the monomer not interacting with F—is positioned with its primary sialic acid binding site exposed above the complex and pointing upwards, in the direction of a putative receptor (FIGS. 15C and 16A). The globular head of the other HN monomer—interacting with F—is rotated 87° with respect to the other monomer's head so that its primary sialic acid binding site is facing laterally (orthogonally from the membrane) and upward in FIGS. 15C and 16A. This suggests an organization where in each HN dimer, one monomer head interacts with F while the other monomer head is free to bind to sialic acid receptor. Significantly, in this cryo-ET structure of the complex on the virus surface, the relationship between the globular heads of the HN monomers differs from that shown in the published crystal structure of the dimer (FIGS. 19A-19C). Compared to the crystal structure of the dimer, the distal monomer head in the cryo-ET structure is rotated 19° about the axis parallel to the membrane (away from the membrane) relative to the monomer head bound to F (the proximal monomer) (FIG. 19A, left). The distal monomer head in the cryo-ET structure is also rotated 48° about the axis perpendicular to the membrane relative to the proximal monomer (FIG. 19A, right). If the monomers are rotated, the crystal and cryo-ET data coincide (FIG. 19B).

The HN dimer interface is important for HN's role in activating F and is key to the signal for fusion upon receptor engagement. Several dimer-interface mutations in HN (HN N551D and HN H552Q) alter HN's activation of F. In a previously published crystal structure of soluble HPIV3 HN, the monomer heads are in tight contact at the secondary binding site that forms at the dimer interface and that is critical for activating F; we showed that a mutation in a residue adjacent to the dimer interface, Q559R, relaxes the HN dimer interface, decreases HN-F interaction and decreases fusion activation. However, the tight conformation of the HN heads in the HN dimer crystal structure also does not fit into our cryo-ET density map, where the HN dimer heads are in a more relaxed conformation. To reconcile the differences between the crystal and cryo-EM structures, it is proposed that a series of rearrangements between the HN monomer heads occur, that include rotation at the HN dimer interface. This raises the possibility that movements around this axis between the HN monomer heads regulate the activation of fusion. To test this hypothesis and determine the functional consequences of this pro-posed flexibility between the globular heads, the resolution of the HN monomer heads (in the HN-complex on the virus surface) in pre-receptor engaged and receptor-engaged states will be refined, for wt HN and HN bearing the specific interface mutations of interest mentioned above (see Table 1) and correlate the structures to function in fusion activation.

Table 1 presents some of the mutant HNs and Fs that will be used. The panel includes well-characterized HNs with head mutations that confer altered neuraminidase, avidity, or F-activation alone or in combination, and F with a mutation that confers delayed activation. The functional consequences of the mutations in Table 1 have been characterized in recombinant viruses (in hand). F with a mutation that decreases F fusion activation will be paired with the HNs variants. These selected examples (and circulating CI complexes) will be compared structurally to test the hypothesis that structural differences in HN-F interaction in the pre-receptor engaged complex impact the complex's function. Here, the pre-fusion HN-F complex will be studied when HN is not receptor-engaged and F is in its pre-triggered state. While it may be challenging to see differences in structures in the pre-fusion static conditions, with 5-8× resolution it is expected to resolve differences, especially those like HN Q559 vs. HN R559 (dimer interface mutation) that have been resolved crystallographically, since the various cryo-EM structures can be superimposed and at this resolution the secondary structure elements clearly will be seen clearly (as for the HN head loops, FIGS. 15A-15E and 18).

### Example 6. Impact of HN Dimer Interface Flexibility: HN Q559R

To determine if flexibility in the HN dimer interface alter the association of HN and F or points of interaction in the HN-F complex, viruses bearing HN Q559R by will be examined by cryo-ET to assess the structural consequence of dimer interface relaxation on the pre-receptor engaged complex on the viral surface. Given the significant difference in the dimer interface seen crystallographically with Q559R8 it is expected to detect a structural difference in the viral surface fusion complex that will be informative about the contribution of the HN dimer interface to HN-F association. The cryo-ET structure shows fewer residue interactions at the dimer interface than predicted by the crystal structure (though H552 remains as an interacting residue), along with the rotation described above (FIGS. 19A-19C), suggesting that there may be rotational movement between the heads that may in turn impact activation of F. This idea will be tested, and the higher resolution will permit to characterize the relationships between the monomer heads.

d. Impact of HN Dimer Interface Flexibility: Cysteine-Stabilized HN

A stabilizing mutation in HN (S554C) was used to obtain irreversible HN-HN interaction (dimer state). However, this single cysteine mutation locks only one point with a disulfide bond, allows for rotation of the HN heads, and does not affect fusion promotion. Therefore, to lock the rotation of the HN heads, it is proposed to generate double and triple cysteine-stabilized HNs. With the single S554C cysteine mutation, it was found that H552Q stabilizes the HN dimer, and Q559R destabilizes the dimer. In our current cryo-ET model, these residues are still in the dimer interface. It is intended now to fully block rotation to ask whether the dimer interface must rotate to activate F. The single cysteine mutation (S554C) will be compared with double (N248C and S233C) and triple cysteine mutations (S554C, N248C, S233C) that should progressively lock the HN dimer and eliminate the freedom of the monomers to rotate. S554 is near the dimer interface of the cryo-ET model. The S233 and N248 are opposite from the dimer interface in the cryo-ET model but are in the HN dimer interface of the crystal structure model (FIG. 19C). The doubly cysteine stabilized HN (N248C and S233C) will lock HN into the structure that is seen in the crystal, narrowing the distance between heads, but would not prevent the heads from tilting with respect to each other, allowing us to explore the effect of constraining the space between the heads. The triply cysteine stabilized HN should also prevent the heads from tilting with respect to each other and allow to explore this structure and reveal the effect of this tilting movement on fusion. To confirm that the correct bonds in expressed HNs have been generated, liquid chromatography mass spec (LC-MS, will be performed on protease digested fragments of purified disulfide bound HNs. The raw data generated by LC-MS will be analyzed using the Mascot search engine to identify disulfide bonded patterns and if correct, recombinant viruses will be engineered bearing singly, doubly, and triply cysteine-stabilized HNs and perform mass spec on HN on the surface of these viruses after lysis and trypsin treatment to determine whether they are the correctly stabilized HNs on the surfaces. It will then be assessed viruses bearing either the single (S554C), double (N248C and S233C) or triple (S554C, S233C, and N248C) cysteine mutated HNs by cryo-ET to understand the effects that a locked dimer has on the pre-receptor engaged HN/F complex structure on the viral surface, to assess the effects of HN dimer stability on the HN heads' conformational shift by comparing the flexible singly cysteine-mutated HN with the triply stabilized HNs, and perform functional assays using the viruses.

The structure at the HN-F interface shown in FIGS. 15 and 18 is new and suggests an integral point of interaction between HN and F This is the first time we propose mutations in these loops of contact but support for their importance includes not only our experimental data but the fact that a mutation in this loop (G387S) emerged in patients with long term persistent HPIV3 infection and that during an HPIV3 outbreak in South Korea during the COVID-19 pandemic, this mutation was found in all cases that were sequenced (n=33).

While the crystal structure of the dimer heads could be thought irrelevant to biological function, it has been previously shown that differences based on this structure reflected HN's activation of F in biological assays8. Therefore, it is proposed that differences between the crystal and cryo-ET structures will represent biologically meaningful differences that will reveal mechanisms of fusion activation.

For biological validation of mutations suggested by structures, it is possible that some mutations will not be viable in viruses if these viruses cannot be generated or cannot grow (cannot evolve to circumvent the lethality). Nonetheless this finding, along with the structural consequence that led there, will be informative.

It is expected to see structures that correlate with functional differences in the pre-receptor engaged state, e.g. HN Q559R, but for some variants it may be impossible to see structural differences in the static complex. Since the variants activate fusion differently, it will capture structural differences in complexes ‘in action’.

A goal is to provide a structural basis for understanding HN-F interactions that stabilize the pre-fusion complex. If defects in the stability of the HN-F complex correlate with premature triggering of F on particles (i.e. inactive particles), it will be observed more F in post-triggered state on those viruses. Interestingly, for measles, it was suggested that recombinants lacking the H head domains (“headless H”) may undergo spontaneous, premature triggering of F to the post-fusion state, resulting in inactivation of the virus. The study of HN-F interactions that stabilize the pre-fusion complex will provide significant new information to address questions that could not be addressed until now; first visualization of the HN-F interaction on the virus prior to receptor engagement, key sites of interaction between HN and F in the complex. Note, even if the resolution of the pre-fusion complex obtainable is not as high as anticipated, by comparing configurations of the HN/F fusion complex using HNs with defined mutations function with structure of the complex will be correlated. Thus, these studies will begin to provide a structural basis for understanding the link between HN-F interaction and virus infectivity.

### Example 7. The HN-F Fusion Complex in Action after Receptor Engagement: Structural Rearrangements of Sequential Transient Intermediates

After HN's receptor engagement, the HN-F complex switches into fusion activation, and F proceeds through a series of transient intermediates. Structural analysis of viral surface fusion complexes with alterations at key sites will elucidate the mechanism of the complex's action.

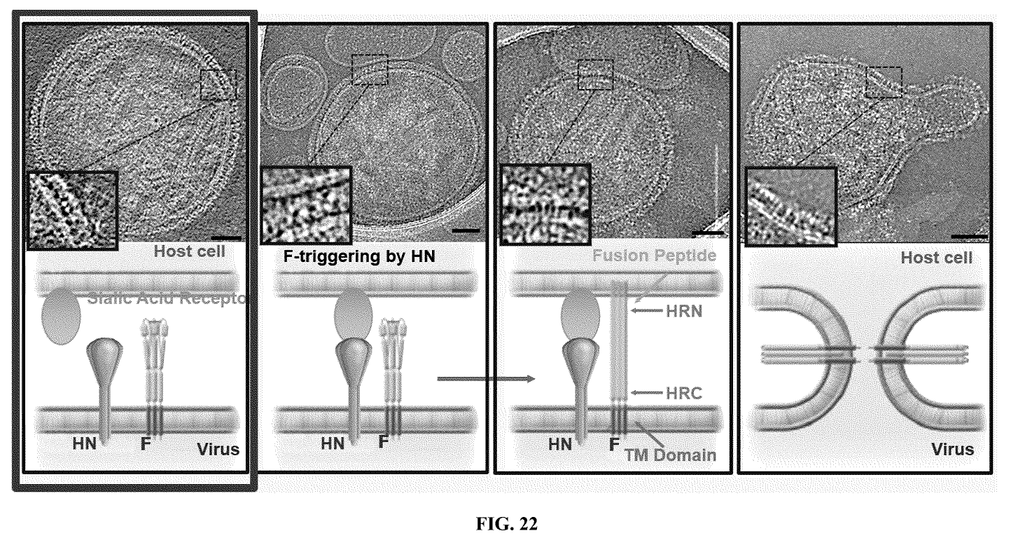

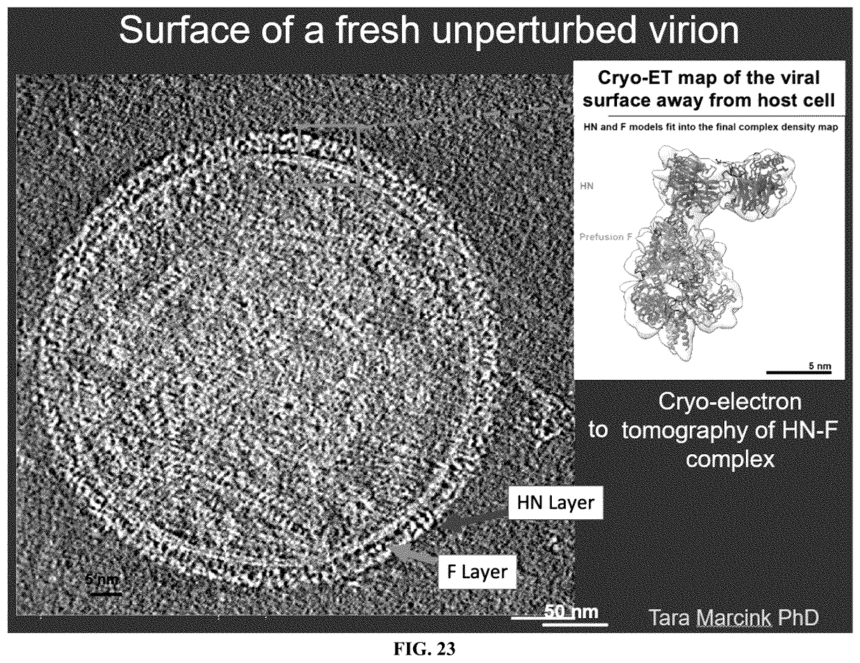

After HN binds receptor it ceases its stabilizing role, and the complex promotes F-mediated fusion. At that final stage, the hydrophobic fusion peptide emerges from its protected site in F and inserts into the target membrane, F rearranges and becomes elongated, before folding back on itself and fusion proceeding to membrane merger (FIG. 22). Focus will be placed on receptor engagement and the transient intermediates of the complex, using well defined mutants (Table 1) to discover the process whereby the complex transitions from pre-fusion stability to fusion promotion during infection. The resolution here is likely to be coarser than that in Example 4—the key will be comparisons between HN-F mutants that affect each stage of the process. These comparisons will reveal the functional correlates of the structures. Sequentially imaging HN-F complexes with an altered receptor avidity, F-activation, HN loop interaction, or HN dimer interface mobility, with measurements of each domain, and then assessing the effect of each alteration on fusion activation/entry, will reveal the mechanisms that underlie the biological impact of these residues. It is only now possible to image the states of the viral glycoprotein complex in situ at the moment of receptor engagement, the relationship of the HN globular heads and their shifts with respect to F after receptor engagement, or the transient intermediate states of the HN/F complex after activation.

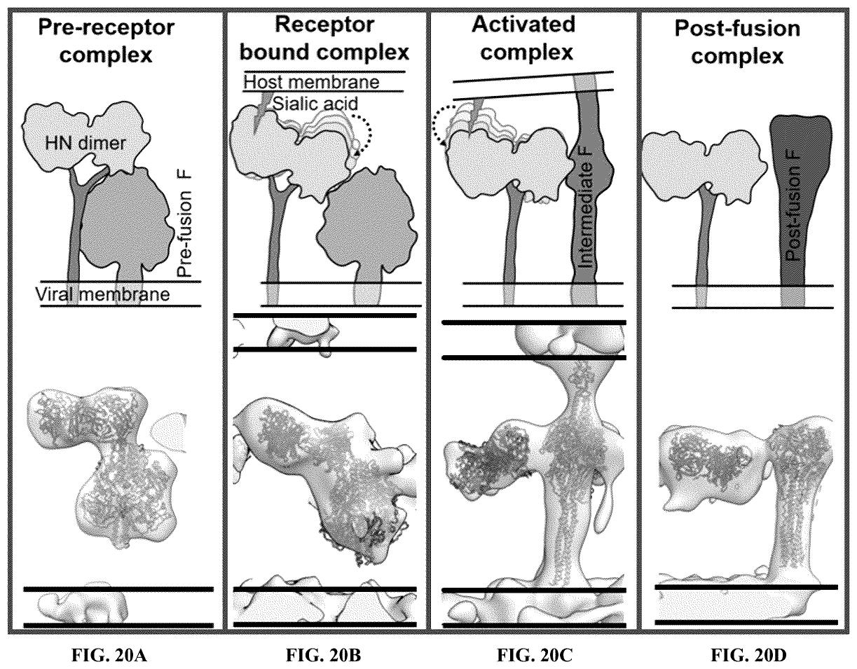

Experimental condition is “Condition A”, target membranes+virus at 4° C. This stage represents binding of HN to cellular receptor (first entry step). For this state of the HN-F complex, we start with the wt CI complex and then carry out the comparisons using the engineered viruses in Table 1. Controls include receptor blockade (with zanamivir/DANA6) or receptor depletion (with neuraminidase treatment.) To assess the geometry of the glycoproteins at areas of virus-target membrane contact we have measured the height of HN and F on the virus. Distant from the virus-target membrane region of contact, HN is approximately 163 Å tall (above the viral surface) and F is 90-120 Å tall, consistent with previous values for HN and pre-fusion F. In regions of contact between viral and target membranes, the average height of HN is 138 Å; 25 Å shorter than the HN's height on the same virion without contact with target membranes. This difference suggests that HN globular domain shortens after receptor engagement. Medium resolution subtomogram averaging (FIGS. 20A-20D) shows that the HN globular head shifts downward, shortening HN (FIG. 20B). This HN head movement results in displacement of the “HN loop” from its pocket at the apex of F (FIGS. 20B and 20C). Further supporting this notion, the distance between the viral target membranes in areas of contact is only 162 Å. HN in the absence of target membrane would not fit in this space without rearrangement. At points of contact, the average distance between the HN heads and the target erythrocyte fragment membrane is 27 Å, a gap that would not be consistent with a conformation of HN constrained by steric clash with the target membrane. To investigate this receptor-bound state and compare the effects of the HN dimer heads' movement for variants with distinct fusion-promotion phenotypes, viruses (variants in Table 1) will be incubated with the target membranes and carry out the comparisons as described. It is hypothesized that this series of positions adopted by the HN-F complex represent movements that occur during the steps of fusion activation (FIGS. 20A-20D). Capturing and resolving the complex at serial steps at physiological temperature will permit us to study the change in interactions within the complex, as we have done for the pre-receptor engaged complex. Defined mutations in the complex will allow us to perturb interactions and study the functional consequences.

e. Impact of HN Dimer Interface Flexibility: HN Q559R8 and the Cysteine-Stabilized HN

Upon receptor engagement, the flexibility at the HN dimer interface affect HN's activation of F will be studied. Comparing the dimer interface mutant HNs with CI HN will reveal the impact of the dimer interface/binding site II on the flexibility of the dimer interface. Recent findings that positioning of the HN globular heads in relation to one another in the cryo-ET structure differs from the existing crystal structure (FIGS. 19A-19C) supports the notion of rotational movement at the interface. These mutants will allow us to assess the effect of more vs. less flexibility at the interface.

f. Impact of the “HN-F Interface Loop”

The virus that emerged during selective pressure of PIA174 antibody treatment bears the mutations HN H552Q and F A194T. This virus will be used to address the impact of the HN head loop/F apex contact zone on the structure of the receptor-engaged complex. In addition, a recombinant virus bearing the HN substituted with the Ab PIA174 loop has been engineered to reveal the consequences of a stronger interaction at that site when the HN/F complex interacts with its receptor. Comparing the results of the effect of flexibility at the interface and the consequences of a stronger interaction at that site when the HN/F complex interacts with its receptor will allow us to distinguish the effects of rotation at the dimer interface from the HN-loop/F-apex interaction during receptor engagement.

It is expected to obtain high-resolution structures of HN-F complexes that permit comparisons of structural information between wt and variants for the receptor engaged (pre-activated) state. Low to medium resolution subtomogram averages have already been obtained and, if difficulty obtaining high resolution HN-F complexes is encountered, viruses may be engineered bearing a stapled F (pre-fusion stabilized F), which should provide highest resolution complex (recombinant viruses bearing stabilized pre-fusion F with wt HN have been generated). The most informative functional complexes for which the highest resolution is available will be chosen to proceed to the examination of activation of fusion. The logic is always to use functional data to guide our structural studies.

g. Response of HN (Complexed with F) to Receptor Engagement: Relationship Between the HN Globular Heads and their Shifts with Respect to F after Receptor Engagement.

Experimental condition is “Condition B”, receptor-bearing target membranes and peptides+virus at 37° C. This condition captures the activated conformation of the HN-F complex before, during and after insertion of F's fusion peptide into the target membrane but without progression to fusion. HN and F interact before receptor engagement and remain engaged during the fusion process (FIG. 20C) separating only after complete membrane merger (FIG. 20D). The focus is on this ongoing interaction between receptor-engaged HN and F during the entire F transition. Variants are used in comparison to the wt CI HN-F complex to assess the impact of HN's binding site I, site II, and stalk, on the continuing interaction of HN with the extended intermediate of F. While it may be difficult to get structural resolution to the level expected for pre-activation complex and extended F, a medium resolution sub-tomogram average has already been obtained and it will be learned by comparing the complexes using experimental conditions of temperature and time to dissect key factors important for HN's activation of F. It is believed that the information to be gained by exploring this new finding—that the shifting of HN heads at the start of the process may signal the transition to fusion activation (FIGS. 20B and 20C) is important for understanding the response of HN to receptor engagement.

In each set of comparisons, both temperature and time are used to help detect differences. F triggering by HN occurs at 37° C. but not at 4° C. and varies between those extremes. The HNs that are most potent activators do so at lower temperatures and faster times. Comparisons between variant and wt CI complexes are made in the presence or absence of receptor blockade (zanamivir or DANA). Once HN triggers F, F-interacting HRC peptides bind to the exposed HR domain of F, blocking F's folding at specific extended stages and trapping intermediate. Peptide to block fusion after F insertion are used (extended intermediate). It was recently shown that the intermediate state of the SARS-CoV-2 spike (S) adopts a range of conformations on the viral surface—a disadvantage for high resolution structure determination. In contrast, HPIV3 F inter-mediates have a more limited range of freedom because the HN/F interaction is maintained throughout activation of the complex, permitting higher resolution structures. To ensure that the intermediates captured by peptide are authentic and to validate the observations select experiments will be performed with a subset of viruses (in hand) that have been shown to be defective in triggering and to complete full fusion only at later time points and higher temperatures; starting with our well-characterized mutants and with recombinant viruses already in hand, including the viruses bearing loop mutations. As the structural analysis suggests domains of interest, viruses will be engineered to study the biological impact of structural findings so that the information flows bidirectionally from biology to structure and back throughout the project.

### Example 8. Impact of HN's Dimer Interface Flexibility: HN Q559R

Addresses whether the flexibility in HN's dimer interface affect interactions between HN's stalk/head and F once HN begins the process of triggering F. Compares dimer interface mutant HN with CI HN will reveal the impact of this site on HN-F's interaction to activate fusion.

a. Impact of Stabilizing the Dimer to Prevent Rotations of the Globular Heads: Cysteine-Stabilized HN

It is aimed to fully block rotation in order to ask if the dimer interface must rotate to activate F. The single cysteine mutation (S554C) will be compared with double and triple cysteine mutations that should progressively lock the HN dimer and eliminate the freedom of the monomers to rotate. The double mutant will allow to explore the effect of constraining the space between the heads; triply cysteine stabilized HN should prevent the heads from tilting with respect to each other and allow to determine the effect of this shift in angle of the heads on fusion, using assays at 37° C. The HN/F pairs and viruses will be prepared and validated. The effects of HN dimer stability on fusion will be assessed, by comparing the three stabilized HNs in fusion assays and perform functional assays using the recombinant viruses as done previously.

b. Impact of the HN-F Interface Site

In light of the new data on the loop of the HN head that contacts F in the pre-receptor engaged HN-F complex (FIGS. 15A-15E), selected HN-F contact loop variants and the A194T mutation in the apex of F will be pursued to investigate the impact of HN head loops/F apex contact on fusion activation by HN. Here, as for any alteration that is introduced because of structural predictions, a key validation is whether the virus evolves to grow in HAE/organoid. It will be assessed whether HN-F contact loop mutations, including the A194T mutation in the apex of F (FIGS. 15A-15E, 16A, and 16B), affect growth in HAE/organoid or whether evolution must take place to permit fitness for airway growth. Evolution of the HN-F complex in human lung will be highly informative about HN-F interaction. Functional analysis will correlate the structural changes with the function of the fusion complex. Depending on the results here viruses will be generated bearing HNs with compensatory mutations that emerge to permit growth in airway and carry out functional analysis and structures of those with the most interesting functional effects.

c. Structural Analysis of the Transient Intermediate States of the HN/F Complex after Activation: Exit from the Pre-Fusion State and Insertion of F into the Target Cell Membrane

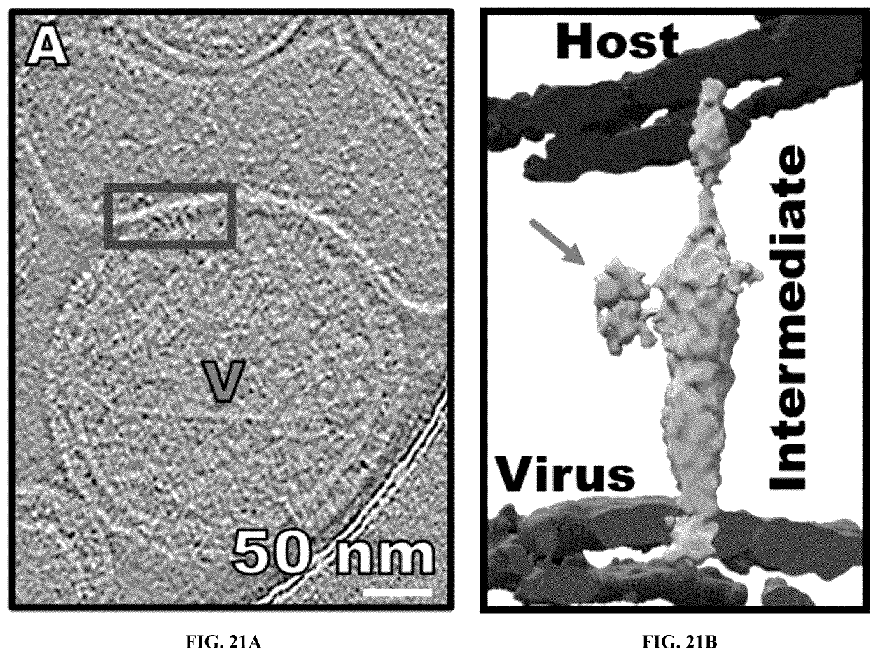

Experimental condition is “Condition B”, receptor-bearing target membranes and peptides+virus at 37° C. This condition will be paired with the tools to capture the transient intermediate states of F before full fusion (FIG. 20C). (Condition C, receptor-bearing cells+virus at 37° C. without peptide, where the fusion process proceeds to completion, will confirm the viability of the fusion complexes on the engineered viruses.) A medium resolution (15-20 Å) intermediate F structure in complex with HN (FIGS. 20C, 21A, and 21B) has been obtained already but the strategy described above will be continued to be applied to obtain a higher resolution view (8-12 Å) of the inter-mediate state of the fusion protein. To ensure intermediates captured by peptide are authentic, each observation is validated using the slow-activating F G396D-bearing virus described in Table 1.

Tools for this structural analysis include the highest avidity/most active triggering HN; peptide inhibitors to lock F in its transient membrane inserted state; controls for binding and activation include zanamivir or DANA to block binding. HPIV3-neutralizing Ab PI174 blocks F-mediated fusion (FIG. 15C), and it is hypothesized that it does so by perturbing HN-F interaction (FIG. 16A), competing with HN for the site on F normally occupied by the 386 HN loop (FIG. 15D), and thereby stabilizes the pre-fusion state of F (FIG. 15E). This will be tested by determining whether Ab PI174 increases the T° of activation of F. Competition for the F epitope between wt HN, the HN substituted with the PIA174 Ab loop, and Ab PI174 will reveal whether the presence of the PIA174 Ab loop blocks the epitope on F; the PIA174 Ab loop on HN should stabilize F and prevent F activation (as the Ab does in FIG. 15E). The F bearing A194T perturbs the HN-F interaction and reduces F activation in the presence of HN but is more readily activated than wt when alone; this variant allows to examine the effect of de-linking activation from HN, via perturbation of the interaction site.

d. Complex of HN T193A H552Q+F

To maximize F-activation and capture of the intermediate states of F, the HN with T193A/H552Q will be used first, our most avid and strongest F-activator HN5. In FIG. 21, complexes on virions were visualized using HRC peptide to lock the transient intermediate of F. Panel B zooms in on the area of virus-target membrane interaction shown in A, where a row of elongated densities can be seen extending from the virus to the target membrane. The density plots show a repeating density 20-30× wide, consistent with the early intermediate cryo-EM structure of influenza. This preliminary subtomogram averaging (FIGS. 20C, 21A, and 21B) provides the first glimpse of the transient intermediate of F and it is anticipated significantly improved resolution. It has been shown that that HN stays engaged throughout the fusion process and direct visual information about this interaction is sought. Subtomogram averaging of the HN-F complex shows this interaction is maintained (FIG. 20C) and focusing on just the intermediate F we see a glimpse of HN interacting with the intermediate F (FIG. 21B). It is anticipated that at 10-15 Å resolution it will be learned more about where HN is in relation to this intermediate state of F, allowing to identify and assess key residues at a new HN-F intermediate interface. Complexes containing the HNs in Table 1 that differ in F-activation will be compared and the HN-F interface variants (HN loop and F apex) to assess the impact of HN's dimer interface, the stalk, and the HN-F contact loop on the continuing interaction of HN with the extended intermediate of F. For example, it will be assessed whether F-A194T, which shows defective activation in the HN-F complex, differs from wt F in its transient intermediates and insertion. Is the F intermediate impaired in formation or does the HN-H552Q mutation overcome this disability of F, a possibility anticipated because these mutations emerged together during selection.

It is possible that intermediates captured with HRC peptide could represent an artifact occurring only in the presence of peptide inhibition. To confirm authenticity of the intermediates, as an alternative to the use of drugs to arrest fusion steps a well-characterized mutant recombinant viruses (in-hand) along with temp. modulation will be used and compare the results to those using peptide to ensure that authentic viral entry steps can be shown, using: (1) virus bearing an HN that has a specific defect in F activation (P111S) and delays progression to fusion; (2) virus bearing a F with a triggering defect making it slower to activate and thereby stalled during the intermediate (F-G396D9, Table 1) (3) zanamivir to block HN-receptor engagement at various time points, to stop the activation process before completion without drugging F. F activation for any of the mutants with temperature and time of exposure is modulated. The peptide blockade approach is the primary strategy to obtain the most homogeneous “population” of extended intermediates for high-resolution cryo-ET structures.

For evaluating the effect of movement at the HN dimer interface, HN Q559R is well studied and will be revealing, and success at obtaining the cysteine-stabilized HNs will provide additional support to the importance of movement of the globular heads.

Additionally, it is noted that the highly active fusion-promoting HN with T193A/H552Q may overwhelm differences between mutant Fs; in this case, we will use wt HN. It is expected T193A/H552Q HN to readily activate all Fs into their intermediates, making it ideal for activating all Fs though less ideal for dissecting differences between Fs.

It is recognized that imaging intermediate states of the HN-F complex is challenging (though encouraged given the already obtained intermediate resolution structures). It has been established conditions for studying HPIV3 with membranes, and isolating fusion complexes. Even in the unlikely outcome that higher resolution F complex intermediate structures from the cryo-ET cannot be obtained, volumes will be obtained into which available structures of HN and F can be modeled.

Focusing on HN, and its role in the complex, follows a logical, comprehensive line of pursuit starting when it was shown that HN and F cooperate to mediate fusion. Regarding F, the focus is on the transient intermediate. However, assessing F in all aspects can be achieved as well as assessing the impact of F mutations, as shown previously, if compensatory mutations in F arise during airway growth.

Previous biological experiments led us to the mechanistic hypotheses and ideal tools for structural analysis of paramyxovirus fusion machinery. Now, a direct picture of the mechanism of action of the HN-F fusion complex on the surface of authentic virions is sought. The ‘in-action’ studies will be advantaged by using the reference information from the static complex structures but are not dependent on those structures. It is expected to capture and characterize the structural transitions that comprise the entry process for this important respiratory virus, during authentic virus-host target membrane interaction. Based on preliminary results, it is anticipated that some of the mechanisms uncovered will be shared across multiple paramyxoviruses and suggest general mechanisms and new targets. This work will be a first on several fronts; high-resolution structures of the actual intact complex of a true clinical isolate fusion complex inserted into an authentic virus and interacting with target membranes. The structural rearrangements that drive a native virus into its target cell will be placed in the context of biological information about properties of the fusion machinery that impact infection in the human. The fusion complexes of these viruses are so finely tuned to infect their specific hosts that any change in their environment spurs quick evolution in the fusion complex. The virus's survival depends on it. By harnessing the features that the virus must maintain, it is expected to gain an unparalleled structural understanding of authentic viral entry.

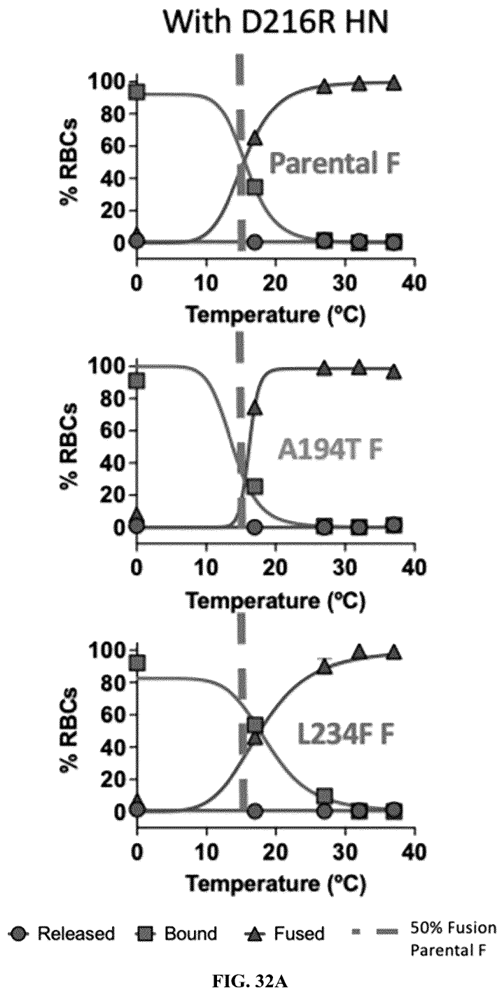

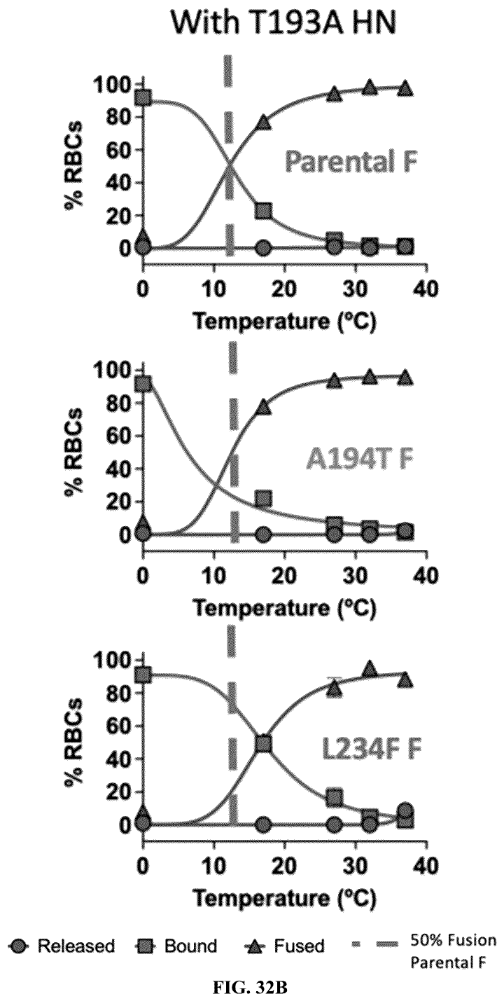

FIG. 32B shows that triggering of F variants by the high avidity HN variant T193A allows constant receptor engagement was assessed at a range of temperatures.

### Example 9. Experimental Procedures

a. Virus Growth and Purification

Recombinant viruses were generated by reverse genetics as previously described using an HPIV3 CI-1-EGFP (enhanced green fluorescent protein) background and a recombinant, clinically isolated (CI-1) HPIV3 virus sequence containing an EGFP cassette between genes P and M. Resulting viruses were propagated using the human airway epithelial (HAE) EpiAirway AIR-100 system (MatTek Corporation) and harvested in 1× phosphate-buffered saline (PBS) with magnesium and calcium (MatTek Corporation). HAE supernatant fluid was subsequently collected and clarified by low-speed centrifugation (180° relative centrifugal force for 10 min at 4° C.). Viruses were titered by limiting dilution infection of Vero cells [the American Type Culture Collection (ATCC)], and infected cells were quantified using an IN Cell Analyzer 2000. All recombinant viruses were sequenced using metagenomic next-generation sequencing (mNGS) as previously described before experimental use.

b. Constructs

Plasmids encoding HPIV3 HN and F were generated through site-directed mutagenesis of a previously constructed pCAGGS mammalian expression vector and sequenced via Sanger sequencing prior to experimental use. Transfections with plasmids were performed in HEK293T cells (ATCC) using Lipofectamine 2000 per the manufacturer's specifications (Invitrogen). Consensus sequence of the laboratory-adapted strain of HPIV3 (Wash/47885/57) used throughout the study was obtained from the NIH (HA-1, NIH no. 47885, catalog no. V323-002-020).

c. Chemicals and Antibodies

Zanamivir (Acme Bioscience) was dissolved in Opti-MEM at a concentration of 50 mM and stored at −80° C. Monoclonal anti-HPIV3 HN antibodies were custom elicited in rats (Aldevron) using eGFP-HN complementary DNA, diluted in Dulbecco's PBS (DPBS) to 100 μg/ml, and kept at 4° C. PIA174 antibody fragment (Fab) and full antibody (Ab) were purchased from Creative Biolabs. PA3/F4 was purified by Rockland Immunochemicals Inc. and was originally generated as described in Bottom-Tanzer et al.

d. Cells

HEK293T cells (ATCC) for transfections were grown in DMEM (Gibco) supplemented with 10% FBS and 1% penicillin-streptomycin (Gibco) at 37° C. and 5% CO₂. Vero cells (ATCC) were grown in DMEM supplemented with 10% FBS and 1% penicillin-streptomycin (Gibco) at 37° C. and 5% CO₂. All cells tested negative for mycoplasma presence using MycoAlert Mycoplasma Detection kit per the manufacturer's specifications (Lonza). Briefly, peptides were produced by standard Fmoc-solid phase methods, and the cholesterol moiety was attached using displacement of an α-bromoamide. Bromoacetyl-PEG4-cholesterol was custom synthesized by Charnwood Molecular (UK). VG-PEG4-chol (5 mM in DMSO) was kept at −20° C.

e. Cryo-ET Preparation HN and F Complex

Lacey carbon gold grids, containing a continuous layer of thin carbon (Ted Pella), were plasma cleaned with Fischione M1070 NanoClean on 70% power for 20 s with a 25% oxygen and 75% argon gas mixture. Eight-microliter drops, containing the anti-HPIV3 HN antibody (100 μg/ml), were incubated on the grids for 10 min, and then, the grids were washed with DPBS to remove unabsorbed antibodies. Next, grids were blotted and placed face down in the HAE supernatant fluid, containing in a six-well plate. Plates were incubated for 30 min at 4° C. with rocking. For the prefusion F Fab bound to prefusion F subtomogram averaging experiments, grids were subjected to another 30-min incubation at 4° C. with rocking in the presence of a PIA174 Fab (10 μg/ml; Creative Biolabs) concentration. After incubation in the supernatant fluid, the grids were washed in cold DPBS 15 times. Grids were then washed in a DPBS solution containing 5-nm gold nanoparticles (Sigma-Aldrich) added at a 10% concentration of the final solution. Grids were then plunge frozen in liquid ethane, using a Vitrobot (Mark IV; Thermo Fisher Scientific Co.). For negative controls, an antibody specific for measles H to the grids was applied and did not observe viral particles on these grids.

f. Cryo-ET Collection for Subtomogram Averaging

Vitrified grids were imaged using a Titan Halo 300-kV transmission electron microscope (Thermo Fisher Scientific Co.) and equipped with a Gatan K3 direct detector and no energy filter. Images were captured at a magnification of ×18,000, giving a pixel size of 1.72 Å at the specimen level. Images were acquired with SerialEM software with a 3.5 to 5.0-μm defocus and a bidirectional tilt series of 3° steps starting from −9° to 51° and then −12° to −51°. Each tilt had a dose of 3.4 e-/A2, resulting in a total dose of ˜100 e-/A2 for the tilt series.

g. Cryo-ET Image Processing

All micrograph movies were aligned using WarpEM. Tilt series were aligned with Etomo, and then, the aligned parameters were used to reconstruct the contrast transfer function (CTF)-corrected tomograms in WarpEM. Eleven tomograms for the HN/F complex and six tomograms for the prefusion F antibody bound to F were selected for subtomogram averaging. Additionally, 12 tomograms for the EV1 HN-F complex, 12 tomograms for the EV2 HN-F complex, and 16 tomograms for the L234F HN-F complex were selected for subtomogram averaging.

h. Cryo-ET Image Processing for the HN/F Complex

The subtomogram averaging process was performed using the Dynamo software package. Subvolumes of the viral particle surfaces (440) A3 were extracted from 2× binned tomograms. The first round of reference free alignment was followed by centering and recropping to (316) A3 on a density corresponding to an HN and F complex. A second round of six iterations with a 360° azimuth range on these centered particles was performed with an initial reference of an HN (PDB ID: 4MZA) and F (PDB ID: 6MJZ) model prefiltered to 30 Å and a cylindrical mask including the membrane. Iterations in rounds 2 to 5 had an initial reference that was low-pass-filtered to 30 Å and came from the previous rounds average. Subvolumes in rounds 3 to 5 (18 iterations) were aligned with decreasing of the azimuth range from 120° to 15° in the presence of cylindrical mask, encompassing only the HN and F density. Overlapping particles with less than 60 Å of separation were removed in the fifth round, and particles with the highest cross correlation were selected for reextraction of subtomograms binned 1× using Warp with a box size of (184) A3. Subvolumes were then subjected to two more rounds of refinement with azimuth range of 15° and 5° steps with a tight mask surrounding HN and F and generated with the mask creation tool in Relion. Subvolumes were subjected to one round of focused refinement with either an HN- or F-focused mask generated in Relion, resulting in a calculated resolution of 10.2 Å at 0.143-Å cutoff value. Resolution for the resulting maps was estimated by FSC with a 0.143-Å cutoff value using three-dimensional FSC (3DFSC). Resolutions were estimated to be 8.5 Å for HN and 9.3 Å for F, and these subtomogram averages were combined for the final map using Chimera. For the HN overview subtomogram averaging, subvolumes of the viral particle surfaces (550) A3 were extracted using the final HN/F complex coordinates from 2× binned tomograms. A round of 12 iterations with a 45° azimuth range on these centered particles was performed with an initial reference that was low-pass-filtered to 30 Å and came from the previous rounds average. Subvolumes were aligned with in the presence of cylindrical mask, encompassing only the HN canopy density. These subvolumes were then subjected to classification using the Dynamo software package through principal components analysis.

i. Cryo-ET Image Processing for the Prefusion F Fab Bound to F

The subtomogram averaging process was performed using the Dynamo software package. Subvolumes of the viral particle surfaces (330) A3 were extracted from 2× binned tomograms. The first round of reference free alignment was followed by a second round of centering all particles to a 30-Å low-pass F (PDB ID: 6MJZ) complex with a resolution limit of 30 Å imposed in each iteration. A cylindrical mask including the membrane was imposed along with a 360° azimuth range. Subvolume alignment in rounds 3 to 5 was implemented with successive decrease of the azimuth range from 120° to 15° in the presence of cylindrical mask, encompassing the prefusion F bound to Fab fragment. Overlapping particles with less than 60 Å of separation were removed in the fifth round, and particles with the highest cross correlation were selected. Subvolumes were then subjected to two more rounds of refinement with azimuth range of 15° and 5° steps with a tight mask surrounding HN and F generated with the mask creation tool in Relion. The final volume obtained had a resolution of 16.51 Å for the complex at the 0.143-Å cutoff value.

j. Model Fitting and Image Analysis

All cryo-EM movie images were visualized using ImageJ, IMOD, ChimeraX, and Chimera. FSCs performed by 3DFSC, Mtriage, and ResMap were used to validate the final resolution. The number of particles included in the analysis and the strategy used are summarized in FIG. 7. Model fitting and model-to-map cross-correlation fit was performed in Chimera and ChimeraX using the protomers from the crystal structure of HN (PDB ID: 4MZA) and cryo-EM structure of F solved with prefusion F Fab antibody fragment (PDB ID: 6MJZ). The axis of rotations for both NDV (PDB IDs: 1E8V and 1E8U) and HPIV3 (PDB ID: 4MZA; cryo-ET model) were calculated and visualized in PyMOL (Schrödinger LLC). Buried surface area in HN dimer interface was calculated in Chimera.

k. AlphaFold2 Model Fitting and Image Analysis

Using ColabFold, a straightforward input complex prediction software for AlphaFold2, the full-length HN sequence was input as a dimer with an output of five structural model PDBs. These PDBs were visualized in ChimeraX. For the stalk region of F, ColabFold was used, and the top scoring trimer was merged with the soluble prefusion F structure using Chimera.

l. Statistics

Statistical analysis was performed where appropriate using GraphPad Prism 9 and two-way analysis of variance (ANOVA). Results are means±SEM unless otherwise stated. P values less than 0.05 were considered statistically significant.

m. Glycoprotein Cross-Linking

Monolayers of human embryonic kidney (HEK) 293T cells transiently expressing HPIV3 F and either HPIV3 HN or empty vector were treated with 2 mM zanamivir in complete medium to prevent cell-cell fusion during overnight incubation at 37° C. After 16 hours, cells were brought to 4° C., incubated for 30 min, with or without DTSSP 1 mM, and then treated with varying concentrations (10, 5, 1, 0.2, 0.04, 0.008, and 0.0016 μg/ml) of PIA174 prefusion F Fab that had been labeled with a Biotium Mix-n-Stain cyanine-based fluorescent (CF) dye antibody labeling kit. After incubation with labeled PIA174 prefusion F Fab for 1 hour on ice, cells were washed, fixed with 4% paraformaldehyde (PFA), treated with DAPI (4′,6-diamidino-2-phenylindole; 1 μg/ml), and imaged on an IN Cell Analyzer 2000 fluorescein isothiocyanate channel. Analysis was performed on a CellProfiler by quantifying the number of green cells divided by the number of DAPI-positive cells. All results were normalized to the value of F alone at the highest concentration.

n. F Stability Assay

To detect the prefusion conformation of F, monolayers of 293T cells transiently expressing HPIV3 F in a 96-well plate were equilibrated at 4° C. for 15 min and then subjected to 55° C. for 5, 15, 30, or 60 min in the absence or presence of PIA174 F Fab (1 μg/ml). For cells not treated with PIA174, the Fab was added after cells were placed on ice. For staining, anti-human H+L (0.5 μg/ml) conjugated with DyLight 594 (Abcam) and DAPI (1 μg/ml; Fisher Scientific Co.) was added. After imaging on Cytation 5 (BioTek), percent positive cells were calculated by automated counting of Fab-positive cells/DAPI-positive cells. Percent positive (prefusion F) was normalized to the value of F alone incubated with Fab at 4° C. To detect the postfusion conformation of F, monolayers of 293T cells transiently expressing HPIV3 F protein in a 96-well plate were equilibrated at 4° C. and then subjected to 55° C. for 30 min in the absence or presence of prefusion PIA174 F antibody (1 μg/ml). Postfusion F was detected with anti-postfusion F antibody [8.5 μg/ml; PA3/F4 (46); purified by Rockland Immunochemicals Inc.] followed by anti-mouse immunoglobulin G (2 μg/ml) conjugated with Alexa Fluor Plus 488 (Invitrogen) mixed with DAPI (1 μg/ml; Fisher Scientific Co.). Fluorescence intensity was determined by imaging on Cytation 5 (BioTek). Total GFP fluorescence intensity was normalized to the value of the no treatment group incubated at 55° C. for 30 min.

HEK 293T cells were transiently transfected with HPIV3 F, HPIV3 F bearing A194T, or HPIV3 F bearing L234F for 18 hours at 32° C. 1 μg/mL of PIA174 FAb was added to half of each plate and the cells were equilibrated to 4° C. for 15 minutes. Cells were either kept at 4° C. or transferred to 55° C. for 5, 15, 30, or 60 minutes. After incubation, the cells were washed and 1 μg/mL of 3x1 mAb was added to all wells for 60 minutes on ice to detect loss of the pre-fusion state. After washing, cells were stained with anti-human H+L conjugated with DyLight 594 (0.5 μg/mL) for 60 minutes at 4° C. Fluorescence intensity was determined by imaging on Cytation 5 (BioTek). The data was normalized such that one-hundred percent pre-fusion for a given F variant and treatment condition (with or without PIA174) represents the fluorescence at 4° C. for that condition.

o. Viral Evolution Experiments

In one experiment, vero cells (ATCC) in a 96-well plate were infected with 200 plaque-forming units (PFUs) per well of HPIV3 expressing mCherry [modified from Afonine et al, a gift from U. Buchholz and P. Collins, National Institute of Allergy and Infectious Diseases] in Opti-MEM (Gibco, 31985070) supplemented with 1% penicillin-streptomycin. After 2 hours, the infection medium was replaced with decreasing concentrations of PIA174 antibody (10 μg/ml; 1:2 dilutions down to 0.04 μg/ml). Because significant viral spread was observed in all wells after 2 days, this virus was passaged onto new cells with four times higher concentration of antibody (40 μg/ml; 1:2 dilutions to 0.16 μg/ml). Infection was monitored, and antibody was replaced every 2 days.

In another experiment, virus was collected and frozen at −80° C. on days 1, 3, 4, 6, and 7 after infection. After day 4 (EV2) and day 7 (EV1), putative escape mutants (observed exponential growth in wells at the highest concentration of antibody) were propagated in Vero cells in the presence of selective concentration of antibody (4 μg/ml) in a T-25 flask. These viruses, and control viruses that were passaged alongside mutant viruses without antibody, were sequenced.

p. Sequencing Library Analysis

Shotgun RNA sequencing metagenomic reads were adapter- and Q20 quality-trimmed using Trimmomatic v0.39. Variants for all samples were called using the reference-based options in LAVA (https://github.com/greninger-lab/lava). Briefly, shotgun RNA sequencing reads for the viral genome from the virus that escaped during the viral evolution experiment were aligned to the reference sequence for the HPIV3 expressing mCherry (GenBank accession no. OP821798) using bwa-mem v0.7.17-r1188, and variant allele frequencies were extracted using bcftools v1.9 and annotated via VarScan v2.3. Sequencing reads are available in NCBI BioProject PRJNA901026.

q. Plaque Reduction Assay

Vero cells (ATCC) in a 96-well plate were infected with 500 PFUs per well of parental or escape variant virus in the presence of a range of concentrations of PIA174 Fab (200 μg/ml; 1:2 dilution to ˜0.2 μg/ml) and incubated for 2 hours at 37° C. Then, the antibody was removed, and the cells overlaid with 0.5% carboxymethyl cellulose. After 18 hours, cells were imaged on a Cytation 5 imager, and fluorescently labeled cells were counted. Inhibition of entry was quantified by comparing the number of infected cells at different concentrations of antibody to the number of infected cells in the absence of antibody for each virus.

r. Antibody Binding

HEK-293T cells were transiently transfected with HPIV3 F or HPIV3 F bearing A194T and incubated for 18 hours at 37° C. The cells were then washed, treated with PIA174 antibody (1 μg/ml) for 1 hour at 4° C., washed again, treated with secondary anti-human conjugated with DyLight 594 (0.5 μg/ml) for 30 min at 4° C., fixed with 4% PFA, and treated with DAPI (2 μg/ml). Cells were then imaged on a Cytation 5 imager, and the total intensity per well was measured.

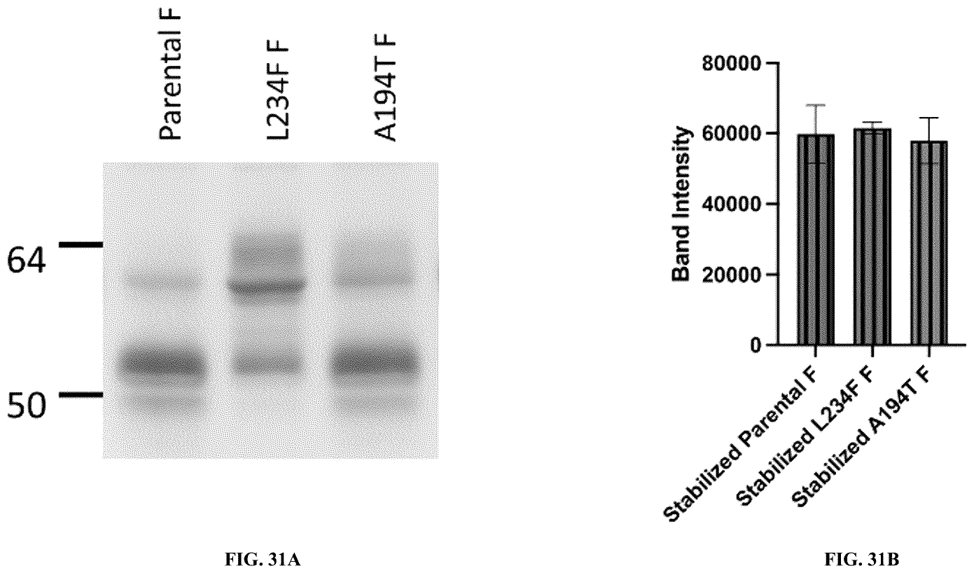

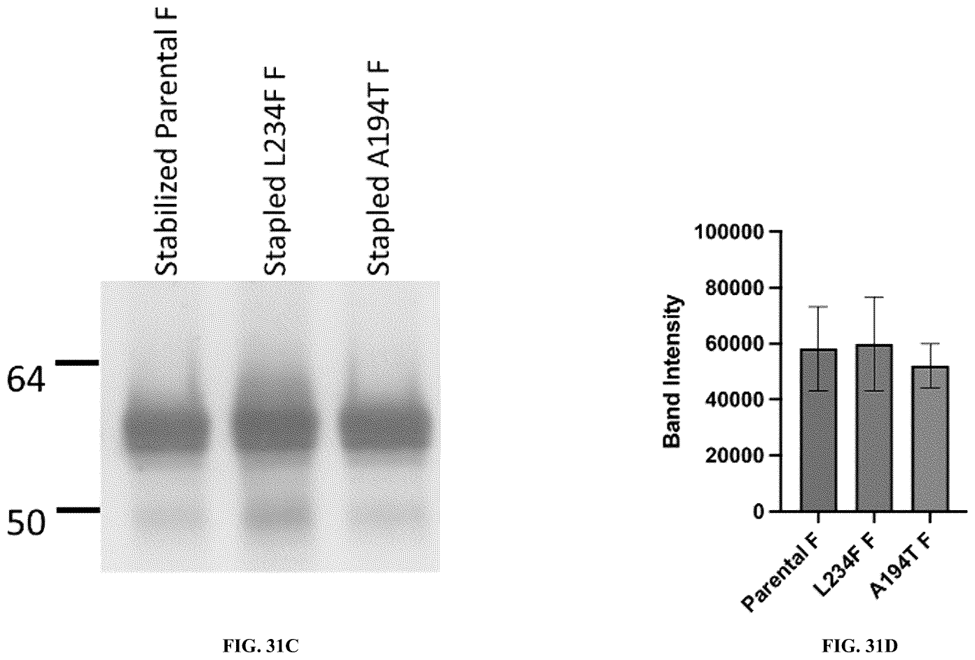

HEK 293T cells were transiently transfected with HPIV3 F, HPIV3 F bearing A194T, or HPIV3 F bearing L234F F with or without additional stabilizing mutations (Q162C, L168C, I213C, G230C, A463V, I474Y) for 18 hours at 32° C. The cells were then washed and treated with PIA174 FAb or 3x1 mAb at a series of concentrations (1 μg/mL; 1:2 dilutions to ˜0.016 μg/mL) for 1 hour at 4° C. The cells were then washed and treated with secondary anti-human conjugated with DyLight 594 (0.5 μg/mL) for 30 minutes at 4° C. The cells were fixed with 4% PFA and treated with 1 μg/mL DAPI. Cells were imaged on a Cytation 5 imager and intensity per well was calculated. Concurrently, cell surface expression of the F proteins was assayed to ensure differences in signal were not attributable to differences in expression (FIGS. 31A and 31B).

### s. β-Gal Complementation-Based Fusion Assay to Assess Antibody Inhibition

Relative fusion was calculated by normalizing all luminescence values to the maximum value in the experiment. Concurrently, cell surface expression of HN (below) was assayed to ensure differences in HN mediated fusion were not attributable to differences in expression.

A fusion assay based on a complementation of β-galactosidase (β-Gal) was previously adapted. In this assay, receptor-bearing cells expressing the omega peptide of β-Gal are mixed with cells coexpressing envelope glycoproteins and the α peptide of β-Gal, and cell fusion leads to complementation. Fusion is stopped by lysing the cells. Substrate was added (Galacton-Star substrate; Applied Biosystems, T1012), and luminescence was read after 1 hour at 500 ms on a Tecan M1000 Pro. Percent inhibition was quantified by comparing relative luminescence units (RLUs) at different concentrations of antibody to RLUs in the absence of antibody 100×[1−(luminescence at X background)/(luminescence in the absence of inhibitor—background)].

t. Conformational Change of Uncleaved F

HEK 293T cells were transiently transfected with HPIV3 HN bearing D216R and uncleaved HPIV3 F, uncleaved HPIV3 F bearing A194T, HPIV3 F bearing L234F, or empty vector (pCAGGs) for 18 hours at 32° C. Cells were treated overnight with 25 mU/well of exogenous Neuraminidase to deplete sialic acid receptors and then washed and incubated with 1% RBC suspensions (pH 7.5) for 30 minutes at 4° C. After the samples were washed to remove unbound RBCs, they were treated with 2 μM of lipid conjugated HRC derived peptide (REF) in CO1 independent medium and brought to 37° C. for 0, 5, 15, 30, or 60 minutes. The plates were rocked, and the liquid phase collected in V-bottom plates for measurement of released RBCs. The cells were incubated with 10 mM zanamivir in CO₂ independent medium to release RBCs that were attached via HN receptor engagement. The liquid phase collected in V-bottom plates for measurement of reversibly bound RBCs. Plates were spun down and pelleted RBCs were lysed in milli-Q water and transferred to a flat bottom 96 well plate for quantitation. The cells were then incubated with RBC lysis solution (ammonium-chloride-potassium lysis buffer; Thermo Fisher Scientific, A1049201), where the lysis of unfused RBCs removes cells that were attached only via F (bound to inhibitory lipopeptide). The liquid phase collected in flat-bottom plates for measurement of irreversibly bound RBCs. The cells were then lysed in 200 μl of dodecyl maltoside HEPES (DH) buffer [5 mM Hepes, 10 mM NaCl, and dodecyl maltoside (0.5 mg/ml)]1:10 in PBS and transferred to flat-bottom 96-well plates for quantification of fused RBCs. Hemoglobin absorbance for the above four compartment was determined by measuring absorbance at 405 nM on a Tecan M1000 Pro. Percentage of irreversibly bound RBCs was calculated by dividing irreversibly bound absorbance by the sum of released, reversibly bound, irreversibly bound, and fused. Results of F variant triggering low neuraminidase HN variant D216R by are shown in FIG. 32A.

u. Measurement of F-Activation and Fusion Between RBCs and Envelope Glycoprotein-Expressing Cells

Monolayers of 293T cells transiently expressing viral glycoproteins (treated overnight with 25 mU per well neuraminidase) were washed and incubated with 1% RBC suspensions (pH 7.5) for 30 min at 4° C. After the samples were rinsed to remove unbound RBCs, they were placed at 37° C. for the indicated time with or without 2 mM zanamivir (pH 8.0). The plates were then rocked, and the liquid phase was collected in V-bottom tubes for measurement of released RBCs. The cells were then incubated at 4° C. with 200 μl of RBC lysis solution (ammonium-chloride-potassium lysis buffer; Thermo Fisher Scientific, A1049201), where the lysis of unfused RBCs removes RBCs that have not fused with cells coexpressing envelope glycoproteins. The liquid phase was collected in V-bottom 96-well plates for measurement of bound RBCs. The cells were then lysed in 200 μl of dodecyl maltoside HEPES (DH) buffer [5 mM Hepes, 10 mM NaCl, and dodecyl maltoside (0.5 mg/ml)]1:10 in PBS and transferred to flat-bottom 96-well plates for quantification of fused RBCs. The amount of RBCs in each of the above three compartments was determined by measuring the absorption at 405 nm.

HEK 293T cells were transiently transfected with HPIV3 HN or uncleaved Influenza HA and HPIV3 F, HPIV3 F bearing A194T, and HPIV3 F bearing L234F, for 18 hours at 32° C. Cells were treated overnight with 25 mU/well of exogenous Neuraminidase to deplete sialic acid receptors and then washed and incubated with 1% RBC suspensions (pH 7.5) for 30 minutes at 4° C. After the samples were washed to remove unbound RBCs, they were kept at 4° C. or brought to 17, 27, or 37° C. for 60 minutes. The plates were rocked, and the liquid phase collected in V-bottom plates for measurement of released RBCs. Plates were spun down and pelleted RBCs were lysed in milli-Q water and transferred to a flat bottom 96 well plate for quantitation. The cells were then incubated with RBC lysis solution (ammonium-chloride-potassium lysis buffer; Thermo Fisher Scientific, A1049201), where the lysis of unfused RBCs removes unfused cells. The liquid phase collected in flat-bottom plates for measurement of irreversibly bound RBCs. The cells were then lysed in 200 μl of dodecyl maltoside HEPES (DH) buffer [5 mM Hepes, 10 mM NaCl, and dodecyl maltoside (0.5 mg/ml)]1:10 in PBS and transferred to flat-bottom 96-well plates for quantification of fused RBCs. Hemoglobin absorbance for the above three compartments was determined by measuring absorbance at 405 nM on a Tecan M1000 Pro.

v. Viral Evolution in HAE

The HAE EpiAirway AIR-100 system (MatTek Corporation) comprises a cultured human-derived tracheo/bronchial epithelium that enables the formation of pseudostratified, differentiated mucociliary epithelium recapitulating in vivo human tissue. Upon receipt from the manufacturer, HAE cultures were transferred to provided 6-well plates containing 2 mL of AIR-100-ASY assay medium (MatTek Corporation) per well with the apical surface remaining exposed to air and incubated at 37° C. in 5% CO₂ overnight prior to infection.

HAE cultures were infected with 500 plaque forming units (PFU) per well of EV1, EV2, and parental virus at the apical surface for 3 hours at 37° C., followed by inoculum removal and incubation at 37° C. for the remainder of the experiment. Provided maintenance medium (MatTek Corporation) was changed every other day with 2 mL medium via the basolateral surface. Viruses were harvested by adding 200 μL of provided 1×PBS containing magnesium and calcium (MatTek Corporation) per well via the apical surface and incubated for 30 minutes at 37° C. Supernatant was subsequently collected and viral titers were determined by limiting dilution infection of Vero cells (ATCC), with infected cells quantified using Cytation 5 (BioTek). These viruses, and control viruses that were passaged alongside mutant viruses without antibody, were sequenced.

w. Cell Surface Biotinylation

HEK 293T cells were transiently transfected with viral glycoprotein variants using conditions of the experiment for which expression was being assayed. Cells were then incubated at 4° C. with 3.3 mM NHS-S-S-dPEG4-biotin (Quanta Biodesign 10194) in DPBS (Gibco 4287080) for 1 hour before lysing with DH Buffer [50 mM HEPES (Gibco Cat #15630080), 100 mM NaCl, 5 mg/mL dodecyl maltoside (Thermo Scientific 89903) in Milli-Q Water] supplemented with complete Protease Inhibitor Cocktail (1 tablet/50 mL; Roche 11836145001). Biotinylated proteins were pulled down with Streptavidin Sepharose (Invitrogen 434341) for 16 hours at 4° C. Protein was eluted from the beads in reducing Laemmli SDS Sample Buffer (Boston BioProducts BP-110R), boiled for 10 minutes, and run on a 4-20% Novex Tris-Glycine Protein Gel (Invitrogen WXP42026BOX). The gel was transferred to nitrocellulose using iBlot quick transfer method (Invitrogen I1323001). The blot was blocked (Invitrogen WB7050), treated with Anti-HIPIV3 F HRC (GenScript; Rabbit) and alkaline phosphatase-conjugated anti-rabbit secondary antibody (Invitrogen WB7105). The blot was then developed using NBT/BCIP Substrate (Invitrogen WP20001). Analysis of the blot was performed in imageJ.

x. Tools and Experimental Conditions for HN Complex in Stable Pre-Fusion State
(1) Red blood cell (RBC) fragments (˜100 nm) bearing sialic acid receptors (“target membranes”);
(2) Peptide fusion inhibitors that bind to the activated F, preventing the progression of fusion;
(3) Anti-HN antibodies to capture virions on grids;
(4) HPIV3 viruses bearing specific HN & F to validate intermediate stages in the complex;
(5) Small molecules (DANA and 4-GU-DANA/zanamivir) that block HN-receptor interaction;
(6) Anti-F antibodies (e.g., PIA17) that neutralize the HN-F fusion mechanism.
(7) Anti-F conformation specific antibodies that recognize either the pre- or post-fusion state of F.
(8) A panel of well-validated biological assays to evaluate the functions of HN and F and the HN-F complex:

Receptor avidity—Established assay in which receptor-bearing cells (RBCs) with different degrees of receptor depletion is used to quantify HN receptor binding.

HN activation of F—Established fusion-triggering assay is used to measure the efficiency of HN's activation of F, or F's “triggerability”, using a range of temperatures (HNs that are more efficient at triggering F do so at lower temperatures).

Full membrane fusion—Quantitative β-galactosidase complementation fusion assay as described above is used.

Stability of F in pre-fusion state—The complementary assays to be used are: (i) F acquisition of protease sensitivity upon receptor engagement at physiological temperature (37° C.); protease-sensitivity means F has initiated conformational changes of activation and (ii) exposure of post-fusion epitopes on F, detected by a conformation specific antibody assay. Only when F is activated by receptor-engaged HN or by heat, the conformation specific Ab (mAb PA3/F3) recognizes post-fusion F. For confirmation, conformational mAb that recognizes the pre-fusion F is used. The mAbs for pre- and post-fusion F are in hand.

HN stabilization of F—The same two assays as for detection of stability of F, now comparing HN-F complexes with different HNs and the same F.

Evaluating biological impact of HN-F mutations on the virus, using viral growth, spread, and evolution in human airway (HAE, organoid)—Mutations that confer a disadvantage will lead to evolution. It has been previously shown that the HN-F fusion complexes of clinical isolates (CI) from humans are not under selective pressure to evolve during growth in HAE or organoids. (CI rapidly adapt to monolayer culture by altering their HN-F fusion complex.) By growing recombinant viruses in HAE, it is determined whether engineered mutations place the virus at a disadvantage and under selective pressure to evolve in the HAE or organoid. Adaptive (compensatory) mutations will be identified by viral genome sequencing. The biology of the virus tells which features of the HN-F complex are relevant to fitness.

Engineered recombinant viruses—For CI viruses that grow only in human lung and cannot be grown in monolayer culture, the CI is transcomplemented with lab-adapted HN and F (codon optimized to minimize the possibility of recombination) while generating the recombinant. Most of the recombinant viruses bearing the HNs indicated in Table 1 are in hand. It has also been shown that virus can be generated bearing non-viable envelope glycoproteins and rescue a “lethal” phenotype virus (not shown), thereby identifying compensatory mutations that allow the virus to exist.

y. Viruses and Target Membrane Vesicles are Incubated Under Experimental Conditions:

Condition 0—Virus at 4° C. alone. This stage represents the step prior to receptor engagement.

Condition A—Receptor-bearing target membranes+virus at 4° C. Viruses bind to the receptors on membranes, but F is not activated at this temperature. This stage represents the first step of entry, binding of HN to a cellular receptor. F maintains a pre-fusion state.

Condition B—Receptor-bearing target membranes and peptides+virus at 37° C. This condition captures the activated conformation of HN-F during and after insertion of F's fusion peptide into the target membrane. It has been shown that HN-receptor interaction triggers F, and the peptide interacts with the N-terminal heptad repeat (HRN) region of F as fusion peptide inserts into the target membrane, retaining F in the ex-tended conformation. To validate intermediates captured by peptides, a set of viruses bearing HN and F with altered fusion properties will be used (delays between steps).

Condition C—Receptor-bearing target membranes+virus at 37° C. This condition captures progress to the post-fusion state. This is a late stage in the fusion process and is included to ensure that in the proposed system, the fusion process proceeds to completion as in nature. The F assumes a post-fusion state. Dependence on receptor engagement will be confirmed using zanamivir or DANA to block HN-receptor interaction (sialic acid receptor depletion with neuraminidase will also be used to confirm authenticity of the steps observed in the presence of small molecules).

Times and temperatures of incubation are chosen based on previous data but will be modified as needed.

### REFERENCES CITED AND INCORPORATED BY REFERENCE

Ader-Ebert, M. Khosravi, M. Herren, M. Avila, L. Alves, F. Bringolf, C. Orvell, J. P. Langedijk, A. Zurbriggen, R. K. Plemper, P. Plattet, Sequential conformational changes in the morbillivirus attachment protein initiate the membrane fusion process. PLOS Pathog. 11, e1004880 (2015). [PMC free article][PubMed][Google Scholar]
Afonine P V, Klaholz B P, Moriarty N W, Poon B K, Sobolev O V, Terwilliger T C, Adams P D, Urzhumtsev A. New tools for the analysis and validation of cryo-EM maps and atomic models. Acta Crystallogr D Struct Biol. 2018; 74(Pt 9):814-40. Epub 2018 Sep. 11. doi: 10.1107/S2059798318009324. PubMed PMID: 30198894; PMCID: PMC6130467.
Ali S O, Takas T, Nyborg A, Shoemaker K, Kallewaard N L, Chiong R, Dubovsky F, Mallory R M. Evaluation of MEDI8852, an anti-influenza A monoclonal antibody, in treating acute uncomplicated influenza. Antimicrob Agents Chemother. 2018; 62(11). Epub 2018 Aug. 29. doi: 10.1128/AAC.00694-18. PubMed PMID: 30150460; PMCID: PMC6201130.
Baker, R. E. Dutch, R. A. Lamb, T. S. Jardetzky, Structural basis for paramyxovirus-mediated membrane fusion. Mol. Cell 3, 309-319 (1999).
Barrett C T, Dutch R E. Viral Membrane Fusion and the Transmembrane Domain. Viruses. 2020; 12(7). Epub 2020 Jul. 2. doi: 10.3390/v12070693. PubMed PMID: 32604992; PMCID: PMC7412173.
Battles, V. Más, E. Olmedillas, O. Cano, M. Vázquez, L. Rodriguez, J. A. Melero, J. S. McLellan, Structure and immunogenicity of pre-fusion-stabilized human metapneumovirus F glycoprotein. Nat. Commun. 8, 1528 (2017).
Benton D J, Gamblin S J, Rosenthal P B, Skehel J J. Structural transitions in influenza haemagglutinin at membrane fusion pH. Nature. 2020; 583(7814):150-3. Epub 2020 May 29. doi: 10.1038/s41586-020-2333-6. PubMed PMID: 32461688; PMCID: PMC7116728.
Bolger A M, Lohse M, Usadel B. Trimmomatic: a flexible trimmer for Illumina sequence data. Bioinformatics. 2014; 30(15):2114-20. Epub 2014 Apr. 4. doi: 10.1093/bioinformatics/btu170. PubMed PMID: 24695404; PMCID: PMC4103590.
Boonyaratanakornkit J, Singh S, Weidle C, Rodarte J, Bakthavatsalam R, Perkins J, Stewart-Jones G B E, Kwong P D, McGuire A T, Pancera M, Taylor J J. Protective antibodies against human parainfluenza virus type 3 infection. MAbs. 2021; 13(1):1912884. Epub 2021 Apr. 21. doi: 10.1080/19420862.2021.1912884. PubMed PMID: 33876699; PMCID: PMC8078717.
Bose, B. D. Welch, C. A. Kors, P. Yuan, T. S. Jardetzky, R. A. Lamb, Structure and mutagenesis of the parainfluenza virus 5 hemagglutinin-neuraminidase stalk domain reveals a four-helix bundle and the role of the stalk in fusion promotion. J. Virol. 85, 12855-12866 (2011).
Bose, T. S. Jardetzky, R. A. Lamba, Timing is everything: Fine-tuned molecular machines orchestrate paramyxovirus entry. Virology 479, 518-531 (2015).
Bottom-Tanzer S F, Rybkina K, Bell I N, Alabi C A, Mathieu C, Lu M, Biswas S, Vasquez M, Porotto M, Melero J A, Mas V, Moscona A. Inhibiting Human Parainfluenza Virus Infection by Preactivating the Cell Entry Mechanism. MBio. 2019; 10(1). doi: 10.1128/mBio.02900-18. PubMed PMID: 30782664; PMCID: PMC6381285.
Bovier F T, Rybkina K, Biswas S, Harder O, Marcink T C, Niewiesk S, Moscona A, Alabi C A, Porotto M. Inhibition of Measles Viral Fusion Is Enhanced by Targeting Multiple Domains of the Fusion Protein. ACS Nano. 2021. Epub 2021 Jul. 23. doi: 10.1021/acsnano.1c02057. PubMed PMID: 34291895.
Brindley M A, Suter R, Schestak I, Kiss G, Wright E R, Plemper R K. A stabilized headless measles virus attachment protein stalk efficiently triggers membrane fusion. J Virol. 2013; 87(21):11693-703. doi: 10.1128/JVI.01945-13. PubMed PMID: 23966411; PMCID: PMC3807326.
Caban M, Rodarte J V, Bibby M, Gray M D, Taylor J J, Pancera M, Boonyaratanakornkit J. Cross-protective antibodies against common endemic respiratory viruses. Nat Commun. 2023; 14(1):798. Epub 2023 Feb. 14. doi: 10.1038/s41467-023-36459-3. PubMed PMID: 36781872; PMCID: PMC9923667 Hutchinson Cancer Center directed to the 3x1 and MxR antibodies. The remaining authors declare no competing interests.
Castano-Diez D, Kudryashev M, Arheit M, Stahlberg H. Dynamo: a flexible, user-friendly development tool for subtomogram averaging of cryo-EM data in high-performance computing environments. J Struct Biol. 2012; 178(2):139-51. Epub 2012 Jan. 17. doi: 10.1016/j.jsb.2011.12.017. PubMed PMID: 22245546.
Castano-Diez D, Kudryashev M, Stahlberg H. Dynamo Catalogue: Geometrical tools and data management for particle picking in subtomogram averaging of cryo-electron tomograms. J Struct Biol. 2017; 197(2):135-44. doi: 10.1016/j.jsb.2016.06.005. PubMed PMID: 27288866.
Chaiwatpongsakorn S, Epand R F, Collins P L, Epand R M, Peeples M E. Soluble respiratory syncytial virus fusion protein in the fully cleaved, pretriggered state is triggered by exposure to low-molarity buffer. J Virol. 2011; 85(8):3968-77. Epub 2011 Feb. 11. doi: JVI.01813-10 [pii]
Chang A, Dutch R E. Paramyxovirus fusion and entry: multiple paths to a common end. Viruses. 2012; 4(4):613-36. Epub 2012 May 17. doi: 10.3390/v4040613. PubMed PMID: 22590688; PMCID: 3347325.
Chang A, Masante C, Buchholz U J, Dutch R E. Human metapneumovirus (HMPV) binding and infection are mediated by interactions between the HMPV fusion protein and heparan sulfate. Journal of virology. 2012; 86(6):3230-43. Epub 2012 Jan. 13. doi: 10.1128/JVI.06706-11. PubMed PMID: 22238303; PMCID: 3302303.
Chang, R. E. Dutch, Paramyxovirus fusion and entry: Multiple paths to a common end. Viruses 4, 613-636 (2012).
Chen Q, Huang X, Wei R, Zhang L, Yin C. Characterization of influenza virus PR8 strain cultured in embryonated eggs by cryo-electron tomography. Biochem Biophys Res Commun. 2019; 516(1):57-62. doi: 10.1016/j.bbrc.2019.05.161. PubMed PMID: 31196621.
Connolly S A, Leser G P, Jardetzky T S, Lamb R A. Bimolecular complementation of paramyxovirus fusion and hemagglutinin-neuraminidase proteins enhances fusion: implications for the mechanism of fusion triggering. Journal of virology. 2009; 83(21):10857-68. Epub 2009 Aug. 28. doi: 10.1128/JVI.01191-09. PubMed PMID: 19710150; PMCID: 2772755.
Connolly S A, Leser G P, Yin H S, Jardetzky T S, Lamb R A. Refolding of a paramyxovirus F protein from prefusion to postfusion conformations observed by liposome binding and electron microscopy. Proc Natl Acad Sci USA. 2006; 103(47):17903-8. Epub 2006 Nov. 8. PubMed PMID: 17093041.
Connolly, G. P. Leser, T. S. Jardetzky, R. A. Lamb, Bimolecular complementation of paramyxovirus fusion and hemagglutinin-neuraminidase proteins enhances fusion: Implications for the mechanism of fusion triggering. J. Virol. 83, 10857-10868 (2009).
Corey L, Gilbert P B, Juraska M, Montefiori D C, Morris L, Karuna S T, Edupuganti S, Mgodi N M, deCamp AC, Rudnicki E, Huang Y, Gonzales P, Cabello R, Orrell C, Lama JR, Laher F, Lazarus E M, Sanchez J, Frank I, Hinojosa J, Sobieszczyk M E, Marshall K E, Mukwekwerere PG, Makhema J, Baden L R, Mullins J I, Williamson C, Hural J, McElrath M J, Bentley C, Takuva S, Gomez Lorenzo M M, Burns D N, Espy N, Randhawa A K, Kochar N, Piwowar-Manning E, Donnell D J, Sista N, Andrew P, Kublin J G, Gray G, Ledgerwood J E, Mascola J R, Cohen M S, Hvtn H, Teams H H S. Two randomized trials of neutralizing antibodies to prevent HIV-1 acquisition. N Engl J Med. 2021; 384(11):1003-14. Epub 2021 Mar. 18. doi: 10.1056/NEJMoa2031738. PubMed PMID: 33730454; PMCID: PMC8189692.
Cox R M, Plemper R K. Structure and organization of paramyxovirus particles. Curr Opin Virol. 2017; 24:105-14. doi: 10.1016/j.coviro.2017.05.004. PubMed PMID: 28601688; PMCID: PMC5529233.
Crennell, T. Takimoto, A. Portner, G. Taylor, Crystal structure of the multifunctional paramyxovirus hemagglutinin-neuraminidase. Nat. Struct. Biol. 7, 1068-1074 (2000). [
Danecek P, Bonfield J K, Liddle J, Marshall J, Ohan V, Pollard M O, Whitwham A, Keane T, McCarthy S A, Davies R M, Li H. Twelve years of SAMtools and BCFtools. Gigascience. 2021; 10(2). Epub 2021 Feb. 17. doi: 10.1093/gigascience/giab008. PubMed PMID: 33590861; PMCID: PMC7931819.
Danecek, J. K. Bonfield, J. Liddle, J. Marshall, V. Ohan, M. O. Pollard, A. Whitwham, T. Keane, S. McCarthy, R. M. Davies, H. Li, Twelve years of SAMtools and BCFtools. Gigascience 10, giab008 (2021).
Das D K, Govindan R, Nikic-Spiegel I, Krammer F, Lemke E A, Munro J B. Direct Visualization of the Conformational Dynamics of Single Influenza Hemagglutinin Trimers. Cell. 2018; 174(4):926-37 e12. doi: 10.1016/j.cell.2018.05.050. PubMed PMID: 29961575; PMCID: PMC6086748.
Deng, Z. Wang, A. M. Mirza, R. M. Iorio, Localization of a domain on the paramyxovirus attachment protein required for the promotion of cellular fusion by its homologous fusion protein spike. Virology 209, 457-469 (1995).
Dirr, I. M. El-Deeb, L. M. G. Chavas, P. Guillon, M. von Itzstein, The impact of the butterfly effect on human parainfluenza virus haemagglutinin-neuraminidase inhibitor design. Sci. Rep. 7, 4507 (2017).
Dodonova S O, Prinz S, Bilanchone V, Sandmeyer S, Briggs J A G. Structure of the Ty3/Gypsy retrotransposon capsid and the evolution of retroviruses. Proc Natl Acad Sci USA. 2019; 116(20):10048-57. doi: 10.1073/pnas.1900931116. PubMed PMID: 31036670; PMCID: PMC6525542.
Domachowske J, Madhi S A, Simoes E A F, Atanasova V, Cabanas F, Furuno K, Garcia-Garcia M L, Grantina I, Nguyen K A, Brooks D, Chang Y, Leach A, Takas T, Yuan Y, Griffin M P, Mankad V S, Villafana T, Group M S. Safety of nirsevimab for RSV in infants with heart or lung disease or prematurity. N Engl J Med. 2022; 386(9):892-4. Epub 2022 Mar. 3. doi: 10.1056/NEJMc2112186. PubMed PMID: 35235733.
Duro, S. Varma, Role of structural fluctuations in allosteric stimulation of paramyxovirus hemagglutinin-neuraminidase. Structure 27, 1601-1611.e2 (2019).
Edupuganti S, Mgodi N, Karuna S T, Andrew P, Rudnicki E, Kochar N, deCamp A, De La Grecca R, Anderson M, Karg C, Tindale I, Greene E, Broder G B, Lucas J, Hural J, Gallardo-Cartagena J A, Gonzales P, Frank I, Sobieszczyk M, Gomez Lorenzo M M, Burns D, Anderson P L, Miner M D, Ledgerwood J, Mascola J R, Gilbert P B, Cohen M S, Corey L, group HHs. Feasibility and successful enrollment in a proof-of-concept HIV prevention trial of VRC01, a broadly neutralizing HIV-1 monoclonal antibody. J Acquir Immune Defic Syndr. 2021; 87(1):671-9. Epub 2021 Feb. 16. doi: 10.1097/QAI.0000000000002639. PubMed PMID: 33587505; PMCID: PMC8397466.
Farzan S, Palermo L M, Yokoyama C C, Orefice G, Fornabaio M, Sarkar A, Kellogg G E, Greengard O, Porotto M, Moscona A. Premature activation of the paramyxovirus fusion protein before target cell attachment: corruption of the viral fusion machinery. J Biol Chem. 2011. Epub 2011 Jul. 30. doi: M111.256248 [pii]
Fernandez P, Trenholme A, Abarca K, Griffin M P, Hultquist M, Harris B, Losonsky G A, Motavizumab Study G. A phase 2, randomized, double-blind safety and pharmacokinetic assessment of respiratory syncytial virus (RSV) prophylaxis with motavizumab and palivizumab administered in the same season. BMC Pediatr. 2010; 10:38. Epub 2010 Jun. 8. doi: 10.1186/1471-2431-10-38. PubMed PMID: 20525274; PMCID: PMC2898783.
Ferrin, UCSF Chimera—A visualization system for exploratory research and analysis. J. Comput. Chem. 25, 1605-1612 (2004). [PubMed][Google Scholar]
Ginsburg A S, Srikantiah P. Respiratory syncytial virus: promising progress against a leading cause of pneumonia. Lancet Glob Health. 2021; 9(12):e1644-e5. Epub 2021 Nov. 15. doi: 10.1016/52214-109X(21)00455-1. PubMed PMID: 34774184; PMCID: PMC8585487.
Go E P, Hua D, Desaire H. Glycosylation and disulfide bond analysis of transiently and stably expressed clade C HIV-1 gp140 trimers in 293T cells identifies disulfide heterogeneity present in both proteins and differences in O-linked glycosylation. J Proteome Res. 2014; 13(9):4012-27. Epub 2014 Jul. 16. doi: 10.1021/pr5003643. PubMed PMID: 25026075; PMCID: PMC4156237.
Goddard T D, Huang C C, Meng E C, Pettersen E F, Couch G S, Morris J H, Ferrin T E. UCSF ChimeraX: Meeting modern challenges in visualization and analysis. Protein Sci. 2018; 27(1):14-25. Epub 2017 Jul. 16. doi: 10.1002/pro.3235. PubMed PMID: 28710774; PMCID: PMC5734306.
Gorman, G.-Y. Chuang, Y.-T. Lai, C.-H. Shen, J. C. Boyington, A. Druz, H. Geng, M. K. Louder, K. M. Kee, R. Rawi, R. Verardi, Y. Yang, B. Zhang, N. A. Doria-Rose, B. Lin, P. L. Moore, L. Morris, L. Shapiro, J. R. Mascola, P. D. Kwong, Structure of super-potent antibody CAP256-VRC26.25 in complex with HIV-1 envelope reveals a combined mode of trimer-apex recognition. Cell Rep. 31, 107488 (2020).
Greengard O, Poltoratskaia N, Leikina E, Zimmerberg J, Moscona A. The anti-influenza virus agent 4-GU-DANA (zanamivir) inhibits cell fusion mediated by human parainfluenza virus and influenza virus HA. J Virol. 2000; 74(23):11108-14. PubMed PMID: 11070006; PMCID: PMC113191.
Greninger A L, Rybkina K, Lin M J, Drew-Bear J, Marcink T C, Shean R C, Makhsous N, Boeckh M, Harder O, Bovier F, Burstein S R, Niewiesk S, Rima B K, Porotto M, Moscona A. Human parainfluenza virus evolution during lung infection of immunocompromised humans promotes viral persistence. J Clin Invest. 2021. Epub 2021 Oct. 6. doi: 10.1172/JCI150506. PubMed PMID: 34609969.
Greninger A L, Rybkina K, Lin M J, Drew-Bear J, Marcink T C, Shean R C, Makhsous N, Boeckh M, Harder O, Bovier F, Burstein S R, Niewiesk S, Rima B K, Porotto M, Moscona A. Human parainfluenza virus evolution during lung infection of immunocompromised humans promotes viral persistence. J Clin Invest. 2021. Epub 2021 Oct. 6. doi: 10.1172/JCI150506. PubMed PMID: 34609969.
Greninger A L, Zerr D M, Qin X, Adler A L, Sampoleo R, Kuypers J M, Englund J A, Jerome K R. Rapid Metagenomic Next-Generation Sequencing during an Investigation of Hospital-Acquired Human Parainfluenza Virus 3 Infections. J Clin Microbiol. 2017; 55(1):177-82. doi: 10.1128/JCM.01881-16. PubMed PMID: 27795347; PMCID: PMC5228228.
Gui L, Jurgens E M, Ebner J L, Porotto M, Moscona A, Lee K K. Electron tomography imaging of surface glycoproteins on human parainfluenza virus 3: association of receptor binding and fusion proteins before receptor engagement. MBio. 2015; 6(1):e02393-14. doi: 10.1128/mBio.02393-14. PubMed PMID: 25691596; PMCID: PMC4337575.
Han A, Czajkowski L, Rosas L A, Cervantes-Medina A, Xiao Y, Gouzoulis M, Lumbard K, Hunsberger S, Reed S, Athota R, Baus H A, Lwin A, Sadoff J, Taubenberger J K, Memoli M J. Safety and efficacy of CR6261 in an influenza A H1N1 healthy human challenge model. Clin Infect Dis. 2021; 73(11):e4260-e8. Epub 2020 Nov. 20. doi: 10.1093/cid/ciaa1725. PubMed PMID: 33211860; PMCID: PMC8664469.
Hashiguchi, Y. Fukuda, R. Matsuoka, D. Kuroda, M. Kubota, Y. Shirogane, S. Watanabe, K. Tsumoto, D. Kohda, R. K. Plemper, Y. Yanagi, Structures of the prefusion form of measles virus fusion protein in complex with inhibitors. Proc. Natl. Acad. Sci. U.S.A. 115, 2496-2501 (2018).
Iketani S, Shean R C, Ferren M, Makhsous N, Aquino D B, des Georges A, Rima B, Mathieu C, Porotto M, Moscona A, Greninger A L. Viral Entry Properties Required for Fitness in Humans Are Lost through Rapid Genomic Change during Viral Isolation. MBio. 2018; 9(4). doi: 10.1128/mBio.00898-18. PubMed PMID: 29970463; PMCID: PMC6030562.
Iorio R M, Melanson V R, Mahon P J. Glycoprotein interactions in paramyxovirus fusion. Future virology. 2009; 4(4):335-51. PubMed PMID: 20161127.
Jumper, R. Evans, A. Pritzel, T. Green, M. Figurnov, O. Ronneberger, K. Tunyasuvunakool, R. Bates, A. Židek, A. Potapenko, A. Bridgland, C. Meyer, S. A. A. Kohl, A. J. Ballard, A. Cowie, B. Romera-Paredes, S. Nikolov, R. Jain, J. Adler, T. Back, S. Petersen, D. Reiman, E. Clancy, M. Zielinski, M. Steinegger, M. Pacholska, T. Berghammer, S. Bodenstein, D. Silver, O. Vinyals, A. W. Senior, K. Kavukcuoglu, P. Kohli, D. Hassabis, Highly accurate protein structure prediction with AlphaFold. Nature 596, 583-589 (2021).
Jurgens E M, Mathieu C, Palermo L M, Hardie D, Horvat B, Moscona A, Porotto M. Measles fusion machinery is dysregulated in neuropathogenic variants. MBio. 2015; 6(1). doi: 10.1128/mBio.02528-14. PubMed PMID: 25670774; PMCID: PMC4337580.
Ke Z, Strauss J D, Hampton C M, Brindley M A, Dillard R S, Leon F, Lamb K M, Plemper R K, Wright E R. Promotion of virus assembly and organization by the measles virus matrix protein. Nat Commun. 2018; 9(1):1736. doi: 10.1038/s41467-018-04058-2. PubMed PMID: 29712906; PMCID: PMC5928126.
Ke, J. Oton, K. Qu, M. Cortese, V. Zila, L. McKeane, T. Nakane, J. Zivanov, C. J. Neufeldt, B. Cerikan, J. M. Lu, J. Peukes, X. Xiong, H.-G. Krausslich, S. H. W. Scheres, R. Bartenschlager, J. A. G. Briggs, Structures and distributions of SARS-CoV-2 spike proteins on intact virions. Nature 588, 498-502 (2020).
Kim H N, Yoon S Y, Lim C S, Lee C K, Yoon J. Phylogenetic analysis of human parainfluenza type 3 virus strains responsible for the outbreak during the COVID-19 pandemic in Seoul, South Korea. J Clin Virol. 2022; 153:105213. Epub 2022 Jun. 21. doi: 10.1016/j.jcv.2022.105213. PubMed PMID: 35724578.
Kim, J. E. Donald, G. Grigoryan, G. P. Leser, A. Y. Fadeev, R. A. Lamb, W. F. DeGrado, Capture and imaging of a prehairpin fusion intermediate of the paramyxovirus PIV5. Proc. Natl. Acad. Sci. U.S.A. 108, 20992-20997 (2011).
Kirchdoerfer, C. A. Cottrell, N. Wang, J. Pallesen, H. M. Yassine, H. L. Turner, K. S. Corbett, B. S. Graham, J. S. McLellan, A. B. Ward, Pre-fusion structure of a human coronavirus spike protein. Nature 531, 118-121 (2016).
Kiss G, Chen X, Brindley M A, Campbell P, Afonso C L, Ke Z, Holl J M, Guerrero-Ferreira R C, Byrd-Leotis L A, Steel J, Steinhauer D A, Plemper R K, Kelly D F, Spearman P W, Wright E R. Capturing enveloped viruses on affinity grids for downstream cryo-electron microscopy applications. Microsc Microanal. 2014; 20(1):164-74. doi: 10.1017/S1431927613013937. PubMed PMID: 24279992; PMCID: PMC4073796.
Koboldt D C, Zhang Q, Larson D E, Shen D, McLellan M D, Lin L, Miller C A, Mardis E R, Ding L, Wilson R K. VarScan 2: somatic mutation and copy number alteration discovery in cancer by exome sequencing. Genome Res. 2012; 22(3):568-76. Epub 2012 Feb. 4. doi: 10.1101/gr.129684.111. PubMed PMID: 22300766; PMCID: PMC3290792.
Kremer J R, Mastronarde D N, McIntosh J R. Computer visualization of three-dimensional image data using IMOD. J Struct Biol. 1996; 116(1):71-6. doi: 10.1006/jsbi.1996.0013. PubMed PMID: 8742726.
Kucukelbir A, Sigworth F J, Tagare H D. Quantifying the local resolution of cryo-EM density maps. Nat Methods. 2014; 11(1):63-5. Epub 2013 Nov. 12. doi: 10.1038/nmeth.2727. PubMed PMID: 24213166; PMCID: PMC3903095.
Lawrence M C, Borg N A, Streltsov V A, Pilling P A, Epa V C, Varghese I N, McKimm-Breschkin J L, Colman P M. Structure of the Haemagglutinin-neuraminidase from Human Parainfluenza Virus Type III. J Mol Biol. 2004; 335(5):1343-57. PubMed PMID: 14729348.
Lee K K. Architecture of a nascent viral fusion pore. EMBO J. 2010; 29(7):1299-311. doi: 10.1038/emboj.2010.13. PubMed PMID: 20168302; PMCID: PMC2857459.
Lee, G. Ozorowski, A. B. Ward, Cryo-EM structure of a native, fully glycosylated, cleaved HIV-1 envelope trimer. Science 351, 1043-1048 (2016). [PMC free article][PubMed][Google Scholar]
Lee, M. L. Fusco, A. J. Hessell, W. B. Oswald, D. R. Burton, E. O. Saphire, Structure of the Ebola virus glycoprotein bound to an antibody from a human survivor. Nature 454, 177-182 (2008).
Lee, R. Andrabi, C. Y. Su, A. Yasmeen, J.-P. Julien, L. Kong, N. C. Wu, R. McBride, D. Sok, M. Pauthner, C. A. Cottrell, T. Nieusma, C. Blattner, J. C. Paulson, P. J. Klasse, I. A. Wilson, D. R. Burton, A. B. Ward, A broadly neutralizing antibody targets the dynamic HIV envelope trimer apex via a long, rigidified, and anionic β-hairpin structure. Immunity 46, 690-702 (2017).
Li Y, Reeves R M, Wang X, Bassat Q, Brooks W A, Cohen C, Moore D P, Nunes M, Rath B, Campbell H, Nair H, Network RSVGE, investigators R. Global patterns in monthly activity of influenza virus, respiratory syncytial virus, parainfluenza virus, and metapneumovirus: a systematic analysis. Lancet Glob Health. 2019; 7(8):e1031-e45. Epub 2019 Jul. 16. doi: 10.1016/52214-109X(19)30264-5. PubMed PMID: 31303294.
Li, W. Li, M. Lu, J. Bess Jr., C. W. Chao, J. Gorman, D. S. Terry, B. Zhang, T. Zhou, S. C. Blanchard, P. D. Kwong, J. D. Lifson, W. Mothes, J. Liu, Subnanometer structures of HIV-1 envelope trimers on aldrithiol-2-inactivated virus particles. Nat. Struct. Mol. Biol. 27, 726-734 (2020).
Liljeroos, M. A. Krzyzaniak, A. Helenius, S. J. Butcher, Architecture of respiratory syncytial virus revealed by electron cryotomography. Proc. Natl. Acad. Sci. U.S.A. 110, 11133-11138 (2013).
Liu, P. Acharya, M. A. Dolan, P. Zhang, C. Guzzo, J. Lu, A. Kwon, D. Gururani, H. Miao, T. Bylund, G.-Y. Chuang, A. Druz, T. Zhou, W. J. Rice, C. Wigge, B. Carragher, C. S. Potter, P. D. Kwong, P. Lusso, Quaternary contact in the initial interaction of CD4 with the HIV-1 envelope trimer. Nat. Struct. Mol. Biol. 24, 370-378 (2017).
Lucic V, Forster F, Baumeister W. Structural studies by electron tomography: from cells to molecules. Annu Rev Biochem. 2005; 74:833-65. Epub 2005 Jun. 15. doi: 10.1146/annurev.biochem.73.011303.074112. PubMed PMID: 15952904.
Lucic V, Yang T, Schweikert G, Forster F, Baumeister W. Morphological characterization of molecular complexes present in the synaptic cleft. Structure. 2005; 13(3):423-34. Epub 2005 Mar. 16. doi: S0969-2126(05)00072-9 [pii]
Mahon P J, Mirza A M, Musich T A, Iorio R M. Engineered intermonomeric disulfide bonds in the globular domain of Newcastle disease virus hemagglutinin-neuraminidase protein: implications for the mechanism of fusion promotion. J Virol. 2008; 82(21):10386-96. Epub 2008 Aug. 30. doi: JVI.00581-08 [pii]
Mangala Prasad, P. Leaman, K. N. Lovendahl, J. T. Croft, M. A. Benhaim, E. A. Hodge, M. B. Zwick, K. K. Lee, Cryo-ET of Env on intact HIV virions reveals structural variation and positioning on the Gag lattice. Cell 185, 641-53.e17 (2022).
Marcink T C, Kicmal T, Armbruster E, Zhang Z, Zipursky G, Golub K L, Idris M, Khao J, Drew-Bear J, McGill G, Gallagher T, Porotto M, des Georges A, Moscona A. Intermediates in SARS-CoV-2 spike-mediated cell entry. Sci Adv. 2022; 8(33):eabo3153. Epub 2022 Aug. 20. doi: 10.1126/sciadv.abo3153. PubMed PMID: 35984891; PMCID: PMC9390989.
Marcink T C, Porotto M, Moscona A. Parainfluenza virus entry at the onset of infection. Adv Virus Res. 2021; 111:1-29. Epub 2021 Oct. 20. doi: 10.1016/bs.aivir.2021.07.001. PubMed PMID: 34663496.
Marcink T C, Wang T, des Georges A, Porotto M, Moscona A. Human parainfluenza virus fusion complex glycoproteins imaged in action on authentic viral surfaces. PLoS Pathog. 2020; 16(9):e1008883. Epub 2020 Sep. 22. doi: 10.1371/journal.ppat.1008883. PubMed PMID: 32956394; PMCID: PMC7529294.
Marcink T C, Yariv E, Rybkina K, Mas V, Bovier F T, des Georges A, Greninger A L, Alabi C A, Porotto M, Ben-Tal N, Moscona A. Hijacking the Fusion Complex of Human Parainfluenza Virus as an Antiviral Strategy. MBio. 2020; 11(1). Epub 2020 Feb. 13. doi: 10.1128/mBio.03203-19. PubMed PMID: 32047132.
Marcink T C, Zipursky G, Cheng W, Steams K, Stenglein S, Golub K, Cohen F, Bovier F, Pfalmer D, Greninger A L, Porotto M, des Georges A, Moscona A. Subnanometer structure of an enveloped virus fusion complex on viral surface reveals new entry mechanisms. Sci Adv. 2023; 9(6):eade2727. Epub 2023 Feb. 11. doi: 10.1126/sciadv.ade2727. PubMed PMID: 36763666; PMCID: PMC9917000.
Mastronarde D N, Held S R. Automated tilt series alignment and tomographic reconstruction in IMOD. J Struct Biol. 2017; 197(2):102-13. doi: 10.1016/j.jsb.2016.07.011. PubMed PMID: 27444392; PMCID: PMC5247408.
Mathieu C, Augusto M T, Niewiesk S, Horvat B, Palermo L M, Sanna G, Madeddu S, Huey D, Castanho M A, Porotto M, Santos N C, Moscona A. Broad spectrum antiviral activity for paramyxoviruses is modulated by biophysical properties of fusion inhibitory peptides. Sci Rep. 2017; 7:43610. doi: 10.1038/srep43610. PubMed PMID: 28344321; PMCID: PMC5361215.
Mattei S, Tan A, Glass B, Muller B, Krausslich H G, Briggs J A G. High-resolution structures of HIV-1 Gag cleavage mutants determine structural switch for virus maturation. Proc Natl Acad Sci USA. 2018; 115(40):E9401-E10. doi: 10.1073/pnas.1811237115. PubMed PMID: 30217893; PMCID: PMC6176557.
Maurer U E, Sodeik B, Grunewald K. Native 3D intermediates of membrane fusion in herpes simplex virus 1 entry. Proc Natl Acad Sci USA. 2008; 105(30):10559-64. Epub 2008 Jul. 26. doi: 0801674105 [pii]
McLellan, M. Chen, M. G. Joyce, M. Sastry, G. B. E. Stewart-Jones, Y. Yang, B. Zhang, L. Chen, S. Srivatsan, A. Zheng, T. Zhou, K. W. Graepel, A. Kumar, S. Moin, J. C. Boyington, G.-Y. Chuang, C. Soto, U. Baxa, A. Q. Bakker, H. Spits, T. Beaumont, Z. Zheng, N. Xia, S.-Y. Ko, J.-P. Todd, S. Rao, B. S. Graham, P. D. Kwong, Structure-based design of a fusion glycoprotein vaccine for respiratory syncytial virus. Science 342, 592-598 (2013).
McLellan, M. Chen, S. Leung, K. W. Graepel, X. du, Y. Yang, T. Zhou, U. Baxa, E. Yasuda, T. Beaumont, A. Kumar, K. Modjarrad, Z. Zheng, M. Zhao, N. Xia, P. D. Kwong, B. S. Graham, Structure of RSV fusion glycoprotein trimer bound to a prefusion-specific neutralizing antibody. Science 340, 1113-1117 (2013).
Metskas L A, Briggs J A G. Fluorescence-Based Detection of Membrane Fusion State on a Cryo-EM Grid using Correlated Cryo-Fluorescence and Cryo-Electron Microscopy. Microsc Microanal. 2019; 25(4):942-9. doi: 10.1017/S1431927619000606. PubMed PMID: 31084637; PMCID: PMC6624127.
Mgodi N M, Takuva S, Edupuganti S, Karuna S, Andrew P, Lazarus E, Garnett P, Shava E, Mukwekwerere P G, Kochar N, Marshall K, Rudnicki E, Juraska M, Anderson M, Karg C, Tindale I, Greene E, Luthuli N, Baepanye K, Hural J, Gomez Lorenzo M M, Burns D, Miner M D, Ledgerwood J, Mascola J R, Donnell D, Cohen M S, Corey L, Team H H. A phase 2b study to evaluate the safety and efficacy of VRC01 broadly neutralizing monoclonal antibody in reducing acquisition of HIV-1 infection in women in sub-saharan Africa: Baseline findings. J Acquir Immune Defic Syndr. 2021; 87(1):680-7. Epub 2021 Feb. 16. doi: 10.1097/QAI.0000000000002649. PubMed PMID: 33587510; PMCID: PMC8436719.
Milligan, Davis, Yu, Ilinykh, Huang, Halfmann, Cross, Borisevich, Agans, Geisbert, Chennareddy, Goff, Piper, Hui, Shaffer, Buck, Heinrich, Branco, Crozier, Holbrook, Kuhn, Kawaoka, Glass, Bukreyev, Geisbert, Worwa, Ahmed, Saphire. Asymmetric and non-stoichiometric glycoprotein recognition by two distinct antibodies results in broad protection against ebolaviruses. Cell 185, 995-1007.e18 (2022).
Moosmann P, Rusconi S. Alpha complementation of LacZ in mammalian cells. Nucleic Acids Res. 1996; 24(6):1171-2. PubMed PMID: 8604354.
Moscona A, Galinski M S. Characterization of human parainfluenza virus type 3 persistent infection in cell culture. J Virol. 1990; 64(7):3212-8. Epub 1990 Jul. 1. doi: 10.1128/JVI.64.7.3212-3218.1990. PubMed PMID: 2161938; PMCID: PMC249533.
Moscona A, Peluso R W. Fusion properties of cells persistently infected with human parainfluenza virus type 3: Participation of hemagglutinin-neuraminidase in membrane fusion. J Virol. 1991; 65:2773-7.
Moscona, R. W. Peluso, Fusion properties of cells persistently infected with human parainfluenza virus type 3: Participation of hemagglutinin-neuraminidase in membrane fusion. J. Virol. 65, 2773-2777 (1991). [PMC free article][PubMed][Google Scholar]
Murrell M, Porotto M, Weber T, Greengard O, Moscona A. Mutations in human parainfluenza virus type 3 HN causing increased receptor binding activity and resistance to the transition state sialic acid analog 4-GU-DANA (zanamivir). J Virol. 2003; 77:309-17.
Navaratnarajah C K, Oezguen N, Rupp L, Kay L, Leonard V H, Braun W, Cattaneo R. The heads of the measles virus attachment protein move to transmit the fusion-triggering signal. Nat Struct Mol Biol. 2011; 18(2):128-34. Epub 2011 Jan. 11. doi: nsmb.1967 [pii]
Navaratnarajah, N. Oezguen, L. Rupp, L. Kay, V. H. J. Leonard, W. Braun, R. Cattaneo, The heads of the measles virus attachment protein move to transmit the fusion-triggering signal. Nat. Struct. Mol. Biol. 18, 128-134 (2011). [PMC free article][PubMed][Google Scholar]
Ngwuta, et al., Prefusion F-specific antibodies determine the magnitude of RSV neutralizing activity in human sera. Sci. Transl. Med. 7, 309ra162 (2015). [PMC free article][PubMed][Google Scholar]
Palermo L, Porotto M, Yokoyama C, Palmer S, Mungall B, Greengard O, Niewiesk S, Moscona A. Human parainfluenza virus infection of the airway epithelium: the viral hemagglutinin-neuraminidase regulates fusion protein activation and modulates infectivity. J Virol. 2009; 83(13):6900-8.
Palermo L M, Porotto M, Greengard O, Moscona A. Fusion promotion by a paramyxovirus hemagglutinin-neuraminidase protein: pH modulation of receptor avidity of binding sites I and II. J Virol. 2007; 81(17):9152-61. PubMed PMID: 17567695.
Palermo L M, Uppal M, Skrabanek L, Zumbo P, Germer S, Toussaint N C, Rima B K, Huey D, Niewiesk S, Porotto M, Moscona A. Features of Circulating Parainfluenza Virus Required for Growth in Human Airway. MBio. 2016; 7(2):e00235. doi: 10.1128/mBio.00235-16. PubMed PMID: 26980833; PMCID: PMC4807361.
Palmer S G, DeVito I, Jenkins S G, Niewiesk S, Porotto M, Moscona A. Circulating clinical strains of human parainfluenza virus reveal viral entry requirements for in vivo infection. J Virol. 2014; 88(22):13495-502. doi: 10.1128/JVI.01965-14. PubMed PMID: 25210187; PMCID: PMC4249073.
Palmer S G, Porotto M, Palermo L M, Cunha L F, Greengard O, Moscona A. Adaptation of human parainfluenza virus to airway epithelium reveals fusion properties required for growth in hosttissue. MBio. 2012; 3(3):e00137-12. Epub 2012 Jun. 7. doi: 10.1128/mBio.00137-12. PubMed PMID: 22669629.
Palmer S G, Porotto M, Palermo L M, Cunha L F, Greengard O, Moscona A. Adaptation of
Palmer, I. DeVito, S. G. Jenkins, S. Niewiesk, M. Porotto, A. Moscona, Circulating clinical strains of human parainfluenza virus reveal viral entry requirements for in vivo infection. J. Virol. 88, 13495-13502 (2014). [PMC free article][PubMed][Google Scholar]
Paterson R G, Hiebert S W, Lamb R A. Expression at the cell surface of biologically active fusion and hemagglutinin/neuraminidase proteins of the paramyxovirus simian virus 5 from cloned cDNA. Proc Natl Acad Sci USA. 1985; 82:7520-4.
Pettersen E F, Goddard T D, Huang C C, Couch G S, Greenblatt D M, Meng E C, Ferrin T E. UCSF Chimera—a visualization system for exploratory research and analysis. J Comput Chem. 2004; 25(13):1605-12. doi: 10.1002/jcc.20084. PubMed PMID: 15264254.
Pettersen E F, Goddard T D, Huang C C, Meng E C, Couch G S, Croll T I, Morris J H, Ferrin T E. UCSF ChimeraX: Structure visualization for researchers, educators, and developers. Protein Sci. 2021; 30(1):70-82. Epub 2020 Sep. 4. doi: 10.1002/pro.3943. PubMed PMID: 32881101; PMCID: PMC7737788.
Plattet P, Plemper R K. Envelope protein dynamics in paramyxovirus entry. MBio. 2013; 4(4). doi: 10.1128/mBio.00413-13. PubMed PMID: 23820396; PMCID: PMC3705453.
Porotto M, Carta P, Deng Y, Kellogg G, Whitt M, Lu M, Mungall B, Moscona A. Molecular determinants of antiviral potency of paramyxovirus entry inhibitors. J Virol. 2007; 81(19):10567-74.
Porotto M, Devito I, Palmer S G, Jurgens E M, Yee J L, Yokoyama C C, Pessi A, Moscona A. Spring-loaded model revisited: Paramyxovirus fusion requires engagement of a receptor binding protein beyond initial triggering of the fusion protein. J Virol. 2011; 85(24):12867-80. Epub 2011 Oct. 7. doi: JVI.05873-11 [pii]
Porotto M, Ferren M, Chen Y W, Siu Y, Makhsous N, Rima B, Briese T, Greninger A L, Snoeck H W, Moscona A. Authentic Modeling of Human Respiratory Virus Infection in Human Pluripotent Stem Cell-Derived Lung Organoids. MBio. 2019; 10(3). doi: 10.1128/mBio.00723-19. PubMed PMID: 31064833; PMCID: PMC6509192.
Porotto M, Fornabaio M, Greengard O, Murrell M T, Kellogg G E, Moscona A. Paramyxovirus receptor-binding molecules: engagement of one site on the hemagglutinin-neuraminidase protein modulates activity at the second site. J Virol. 2006; 80(3):1204-13. PubMed PMID: 16414997.
Porotto M, Fornabaio M, Kellogg G E, Moscona A. A second receptor binding site on human parainfluenza virus type 3 hemagglutinin-neuraminidase contributes to activation of the fusion mechanism. Journal of virology. 2007; 81(7):3216-28. Epub 2007 Jan. 19. doi: 10.1128/JVI.02617-06. PubMed PMID: 17229690; PMCID: 1866072.
Porotto M, Greengard O, Poltoratskaia N, Horga M A, Moscona A. Human parainfluenza virus type 3 HN-receptor interaction: effect of 4-guanidino-Neu5Ac2en on a neuraminidase-deficient variant. J Virol. 2001; 75(16):7481-8. doi: 10.1128/JVI.75.16.7481-7488.2001. PubMed PMID: 11462020; PMCID: PMC114983.
Porotto M, Murrell M, Greengard O, Doctor L, Moscona A. Influence of the human parainfluenza virus 3 attachment protein's neuraminidase activity on its capacity to activate the fusion protein. J Virol. 2005; 79(4):2383-92.
Porotto M, Murrell M, Greengard O, Moscona A. Triggering of human parainfluenza virus 3 fusion protein(F) by the hemagglutinin-neuraminidase (HN): an HN mutation diminishing the rate of F activation and fusion. J Virol. 2003; 77(6):3647-54.
Porotto M, Palmer S G, Palermo L M, Moscona A. Mechanism of fusion triggering by human parainfluenza virus type III: communication between viral glycoproteins during entry. J Biol Chem. 2012; 287(1):778-93. Epub 2011 Nov. 24. doi: 10.1074/jbc.M111.298059. PubMed PMID: 22110138; PMCID: 3249132.
Porotto M, Rockx B, Yokoyama C, Talekar A, DeVito I, Palermo L, Liu J, Cortese R, Lu M, Feldmann H, Pessi A, Moscona A. Inhibition of Nipah Virus Infection In Vivo: Targeting an Early Stage of Paramyxovirus Fusion Activation during Viral Entry. PLoS Pathog. 2010; 6 (10) e1001168 doi:10.1371/journal.ppat.1001168.
Porotto M, Salah Z, Devito I, Talekar A, Palmer S G, Xu R, Wilson I A, Moscona A. The second receptor binding site of the globular head of the Newcastle disease virus (NDV) hemagglutinin-neuraminidase activates the stalk of multiple paramyxovirus receptor binding proteins to trigger fusion. J Virol. 2012; 86(10):5730-41. Epub 2012 Mar. 23. doi: 10.1128/JVI.06793-11. PubMed PMID: 22438532.
Porotto M, Salah Z W, Gui L, Devito I, Jurgens E M, Lu H, Yokoyama C C, Palermo L M, Lee K K, Moscona A. Regulation of paramyxovirus fusion activation: the hemagglutinin-neuraminidase protein stabilizes the fusion protein in a pretriggered state. Journal of virology. 2012; 86(23):12838-48. Epub 2012 Sep. 21. doi: 10.1128/JVI.01965-12. PubMed PMID: 22993149; PMCID: 3497673.
Porotto M, Yi F, Moscona A, LaVan D A. Synthetic protocells interact with viral nanomachinery and inactivate pathogenic human virus. PLoS One. 2011; 6(3):e16874. Epub 2011 Mar. 11. doi: 10.1371/journal.pone.0016874. PubMed PMID: 21390296; PMCID: 3046955.
Porotto M, Yokoyama C, Orefice G, Kim H-S, Moscona A. Kinetic dependence of paramyxovirus entry inhibition. J Virol. 2009; 83(13):6947-51.
Qu K, Glass B, Dolezal M, Schur F K M, Murciano B, Rein A, Rumlova M, Ruml T, Krausslich H G, Briggs J A G. Structure and architecture of immature and mature murine leukemia virus capsids. Proc Natl Acad Sci USA. 2018; 115(50):E11751-E60. doi: 10.1073/pnas.1811580115. PubMed PMID: 30478053; PMCID: PMC6294937.
Rodrigues Toste de Carvalho A L, Liu H Y, Chen Y W, Porotto M, Moscona A, Snoeck H W. The in vitro multilineage differentiation and maturation of lung and airway cells from human pluripotent stem cell-derived lung progenitors in 3D. Nat Protoc. 2021; 16(4):1802-29. Epub 2021 Mar. 3. doi: 10.1038/s41596-020-00476-z. PubMed PMID: 33649566.
Rossey, M. S. A. Gilman, S. C. Kabeche, K. Sedeyn, D. Wrapp, M. Kanekiyo, M. Chen, V. Mas, J. Spitaels, J. A. Melero, B. S. Graham, B. Schepens, J. S. McLellan, X. Saelens, Potent single-domain antibodies that arrest respiratory syncytial virus fusion protein in its prefusion state. Nat. Commun. 8, 14158 (2017). [PMC free article][PubMed][Google Scholar]
Russell C J, Kantor K L, Jardetzky T S, Lamb R A. A dual-functional paramyxovirus F protein regulatory switch segment: activation and membrane fusion. J Cell Biol. 2003; 163(2):363-74. PubMed PMID: 14581458.
San-Juan-Vergara H, Sampayo-Escobar V, Reyes N, Cha B, Pacheco-Lugo L, Wong T, Peeples M E, Collins P L, Castano M E, Mohapatra S S. Cholesterol-rich Microdomains as Docking Platforms for Respiratory Syncytial Virus in Normal Human Bronchial Epithelial Cells. J Virol. 2011. Epub 2011 Nov. 18. doi: JVI.06274-11 [pii]10.1128/JVI.06274-11. PubMed PMID: 22090136.
Scheid, P. Choppin, Identification of biological activities of paramyxovirus glycoproteins. Activation of cell fusion, hemolysis, and infectivity by proteolytic cleavage of an inactive precursor protein of Sendai virus. Virology 57, 475-490 (1974). [PubMed][Google Scholar]
Scheres S H. A Bayesian view on cryo-EM structure determination. J Mol Biol. 2012; 415(2):406-18. Epub 2011 Nov. 22. doi: 10.1016/j.jmb.2011.11.010. PubMed PMID: 22100448; PMCID: PMC3314964.
Scheres S H. RELION: implementation of a Bayesian approach to cryo-EM structure determination. J Struct Biol. 2012; 180(3):519-30. Epub 2012 Sep. 25. doi: 10.1016/j.jsb.2012.09.006. PubMed PMID: 23000701; PMCID: PMC3690530.
Schildgen V, van den Hoogen B, Fouchier R, Tripp R A, Alvarez R, Manoha C, Williams J, Schildgen O. Human Metapneumovirus: lessons learned over the first decade. Clin Microbiol Rev. 2011; 24(4):734-54. Epub 2011 Oct. 7. doi: 10.1128/CMR.00015-11
Schowalter R M, Chang A, Robach J G, Buchholz U J, Dutch R E. Low-pH triggering of human metapneumovirus fusion: essential residues and importance in entry. J Virol. 2009; 83(3):1511-22. Epub 2008 Nov. 28. doi: JVI.01381-08 [pii]10.1128/JVI.01381-08. PubMed PMID: 19036821; PMCID: 2620907.
Shi T, Arnott A, Semogas I, Falsey A R, Openshaw P, Wedzicha J A, Campbell H, Nair H, Investigators R. The Etiological Role of Common Respiratory Viruses in Acute Respiratory Infections in Older Adults: A Systematic Review and Meta-analysis. J Infect Dis. 2020; 222(Supplement_7):S563-S9. Epub 2019 Mar. 9. doi: 10.1093/infdis/jiy662. PubMed PMID: 30849176; PMCID: PMC7107439.
Shi T, McLean K, Campbell H, Nair H. Aetiological role of common respiratory viruses in acute lower respiratory infections in children under five years: A systematic review and meta-analysis. J Glob Health. 2015; 5(1):010408. Epub 2015 Oct. 9. doi: 10.7189/jogh.05.010408. PubMed PMID: 26445672; PMCID: PMC4593292. Falsey A R, Hennessey P A, Formica M A, Cox C, Walsh E E. Respiratory syncytial virus infection in elderly and high-risk adults. The New England journal of medicine. 2005; 352(17):1749-59. PubMed PMID: 15858184.
Stewart-Jones G B E, Chuang G Y, Xu K, Zhou T, Acharya P, Tsybovsky Y, Ou L, Zhang B, Fernandez-Rodriguez B, Gilardi V, Silacci-Fregni C, Beltramello M, Baxa U, Druz A, Kong W P, Thomas P V, Yang Y, Foulds K E, Todd J P, Wei H, Salazar A M, Scorpio D G, Carragher B, Potter C S, Corti D, Mascola J R, Lanzavecchia A, Kwong P D. Structure-based design of a quadrivalent fusion glycoprotein vaccine for human parainfluenza virus types 1-4. Proc Natl Acad Sci USA. 2018; 115(48):12265-70. doi: 10.1073/pnas.1811980115. PubMed PMID: 30420505; PMCID: PMC6275507.
Stobart, C. A. Rostad, Z. Ke, R. S. Dillard, C. M. Hampton, J. D. Strauss, H. Yi, A. L. Hotard, J. Meng, R. J. Pickles, K. Sakamoto, S. Lee, M. G. Currier, S. M. Moin, B. S. Graham, M. S. Boukhvalova, B. E. Gilbert, J. C. G. Blanco, P. A. Piedra, E. R. Wright, M. L. Moore, A live RSV vaccine with engineered thermostability is immunogenic in cotton rats despite high attenuation. Nat. Commun. 7, 13916 (2016). [PMC free article][PubMed][Google Scholar]
Talekar A, DeVito I, Salah Z, Palmer S G, Chattopadhyay A, Rose J K, Xu R, Wilson I A, Moscona A, Porotto M. Identification of a region in the stalk domain of the nipah virus receptor binding protein that is critical for fusion activation. J Virol. 2013; 87(20):10980-96. doi: 10.1128/JVI.01646-13. PubMed PMID: 23903846; PMCID: PMC3807285.
Talekar A, Moscona A, Porotto M. Measles virus fusion machinery activated by sialic acid binding globular domain. J Virol. 2013; 87(24):13619-27. doi: 10.1128/JVI.02256-13. PubMed PMID: 24109225; PMCID: PMC3838214.
Tan Y Z, Baldwin P R, Davis J H, Williamson J R, Potter C S, Carragher B, Lyumkis D. Addressing preferred specimen orientation in single-particle cryo-EM through tilting. Nat Methods. 2017; 14(8):793-6. Epub 2017 Jul. 4. doi: 10.1038/nmeth.4347. PubMed PMID: 28671674; PMCID: PMC5533649.
Tanabayashi, R. W. Compans, Functional interaction of paramyxovirus glycoproteins: Identification of a domain in Sendai virus HN which promotes cell fusion. J. Virol. 70, 6112-6118 (1996).
Tegunov D, Cramer P. Real-time cryo-electron microscopy data preprocessing with Warp. Nat Methods. 2019; 16(11):1146-52. Epub 2019 Oct. 9. doi: 10.1038/s41592-019-0580-y. PubMed PMID: 31591575; PMCID: PMC6858868.
Tegunov D, Xue L, Dienemann C, Cramer P, Mahamid J. Multi-particle cryo-EM refinement with M visualizes ribosome-antibiotic complex at 3.5 A in cells. Nat Methods. 2021; 18(2):186-93. Epub 2021 Feb. 6. doi: 10.1038/s41592-020-01054-7. PubMed PMID: 33542511.
Tian, M. B. Battles, S. M. Moin, M. Chen, K. Modjarrad, A. Kumar, M. Kanekiyo, K. W. Graepel, N. M. Taher, A. L. Hotard, M. L. Moore, M. Zhao, Z.-Z. Zheng, N.-S. Xia, J. S. McLellan, B. S. Graham, Structural basis of respiratory syncytial virus subtype-dependent neutralization by an antibody targeting the fusion glycoprotein. Nat. Commun. 8, 1877 (2017).
Turoňová, M. Sikora, C. Schürmann, W. J. H. Hagen, S. Welsch, F. E. C. Blanc, S. von Bulow, M. Gecht, K. Bagola, C. Hörner, G. van Zandbergen, J. Landry, N. T. D. de Azevedo, S. Mosalaganti, A. Schwarz, R. Covino, M. D. Mühlebach, G. Hummer, J. K. Locker, M. Beck, In situ structural analysis of SARS-CoV-2 spike reveals flexibility mediated by three hinges. Science 370, 203-208 (2020).
Varadi, S. Anyango, M. Deshpande, S. Nair, C. Natassia, G. Yordanova, D. Yuan, O. Stroe, G. Wood, A. Laydon, A. Židek, T. Green, K. Tunyasuvunakool, S. Petersen, J. Jumper, E. Clancy, R. Green, A. Vora, M. Lutfi, M. Figurnov, A. Cowie, N. Hobbs, P. Kohli, G. Kleywegt, E. Birney, D. Hassabis, S. Velankar, AlphaFold protein structure database: Massively expanding the structural coverage of protein-sequence space with high-accuracy models. Nucleic Acids Res. 50, D439-D444 (2022).
Wang, M. Amaya, A. Addetia, H. V. Dang, G. Reggiano, L. Yan, A. C. Hickey, F. DiMaio, C. C. Broder, D. Veesler, Architecture and antigenicity of the Nipah virus attachment glycoprotein. Science 375, 1373-1378 (2022).
Wrapp, N. Wang, K. S. Corbett, J. A. Goldsmith, C.-L. Hsieh, O. Abiona, B. S. Graham, J. S. McLellan, Cryo-EM structure of the 2019-nCoV spike in the prefusion conformation. Science 367, 1260-1263 (2020).
Xu R, Palmer S G, Porotto M, Palermo L M, Niewiesk S, Wilson I A, Moscona A. Interaction between the hemagglutinin-neuraminidase and fusion glycoproteins of human parainfluenza virus type III regulates viral growth in vivo. MBio. 2013; 4(5):e00803-13. doi: 10.1128/mBio.00803-13. PubMed PMID: 24149514; PMCID: PMC3812707.
Yao, Y. Song, Y. Chen, N. Wu, J. Xu, C. Sun, J. Zhang, T. Weng, Z. Zhang, Z. Wu, L. Cheng, D. Shi, X. Lu, J. Lei, M. Crispin, Y. Shi, L. Li, S. Li, Molecular architecture of the SARS-CoV-2 virus. Cell 183, 730-738.e13 (2020).
Yin H S, Paterson R G, Wen X, Lamb R A, Jardetzky T S. Structure of the uncleaved ectodomain of the paramyxovirus (hPIV3) fusion protein. Proc Natl Acad Sci USA. 2005; 102(26):9288-93. PubMed PMID: 15964978.
Yin, X. Wen, R. G. Paterson, R. A. Lamb, T. S. Jardetzky, Structure of the parainfluenza virus 5 F protein in its metastable, prefusion conformation. Nature 439, 38-44 (2006).
Zaitsevet, M. von Itzstein, D. Groves, M. Kiefel, T. Takimoto, A. Portner, G. Taylor, Second sialic acid binding site in newcastle disease virus hemagglutinin-neuraminidase: Implications for fusion. J. Virol. 78, 3733-3741 (2004).
Zhang L, Bukreyev A, Thompson C I, Watson B, Peeples M E, Collins P L, Pickles R J. Infection of ciliated cells by human parainfluenza virus type 3 in an in vitro model of human airway epithelium. J Virol. 2005; 79(2):1113-24. PubMed PMID: 15613339.
Zhang S, Go E P, Ding H, Anang S, Kappes J C, Desaire H, Sodroski J G. Analysis of Glycosylation and Disulfide Bonding of Wild-Type SARS-CoV-2 Spike Glycoprotein. J Virol. 2022; 96(3):e0162621. Epub 2021 Nov. 25. doi: 10.1128/JVI.01626-21. PubMed PMID: 34817202; PMCID: PMC8827021.

We claim:

1. A method of screening for a therapeutic agent that inhibits membrane fusion by a Paramyxoviridae virus, the method comprising:
administering a small molecule or peptide to a receptor binding protein of the Paramyxoviridae virus or a fragment thereof, wherein the fragment of the receptor binding protein comprises globular head domain or a fragment thereof, wherein the globular head domain caps the fusion (F) protein of the Paramyxoviridae virus at its apex;
and/or
administering a small molecule or peptide to a fragment of a fusion (F) protein of the Paramyxoviridae virus comprising the apex of the F protein,
wherein binding of the small molecule or peptide to the receptor binding protein of the Paramyxoviridae virus or a fragment thereof or to the apex of the F protein of the Paramyxoviridae virus or a fragment thereof indicates the small molecule or peptide is likely a therapeutic agent that inhibits membrane fusion by the Paramyxoviridae virus.

2. The method of claim 1, wherein the globular head domain of the receptor binding protein caps the F protein at its apex in its pre-fusion configuration.

3. The method of claim 1, wherein the fragment of the receptor binding protein comprises a loop and/or a beta sheet.

4. The method of claim 1, wherein the fragment of the receptor binding protein comprises loop and the loop comprises an amino acid sequence set forth in: KGLNSVOK (SEQ ID NO. 1), LSLTVELK (SEQ ID NO. 2), LSDGENPK (SEQ ID NO. 3), LSLGGDII (SEQ ID NO. 4), LRQDLQTN (SEQ ID NO. 5), or KVSTSLGE (SEQ ID NO. 6).

5. The method of claim 1, wherein the apex of the F protein comprises an amino acid sequence selected from the group consisting of: LFLEAAGLQ (SEQ ID NO. 7), NELIPSMNQ (SEQ ID NO. 8), LVPTIDKI (SEQ ID NO. 9), TNLVPSIDQ (SEQ ID NO. 10), QDHINSV (SEQ ID NO. 11), and ISNIE (SEQ ID NO. 12).

6. The method of claim 1, wherein the Paramyxoviridae virus is selected from the group consisting of: measles virus, Nipah virus, Hendra virus, Newcastle disease virus, and parainfluenza virus.

7. The method of claim 6, wherein the parainfluenza virus is human parainfluenza virus 3 or parainfluenza virus 5.

8. A method of generating a neutralizing antibody against a Paramyxoviridae virus, the method comprising:
immunizing an animal with a polypeptide comprising an amino acid sequence encoding at least a portion of a globular head domain of a receptor binding protein of the Paramyxoviridae virus, wherein the globular head domain caps the fusion (F) protein of the Paramyxoviridae virus at its apex; or
immunizing an animal with a polypeptide comprising an amino acid sequence encoding a fragment of fusion (F) protein of the Paramyxoviridae virus, wherein the fragment of F protein comprises its apex.

9. The method of claim 8, wherein the globular head domain of the receptor binding protein caps the F protein in its pre-fusion configuration.

10. The method of claim 8, wherein the at least one portion of a globular head domain of the receptor binding protein comprises a loop and/or a beta sheet.

11. The method of claim 8, wherein the polypeptide comprises an amino acid sequence set forth in SEQ ID NOs 1-6.

12. A method of inhibiting membrane fusion of a Paramyxoviridae virus to a target cell, the method comprising:
administering to the target cell a therapeutic peptide or protein comprising a globular head domain of a receptor binding protein of the Paramyxoviridae virus or a fragment thereof, wherein the globular head domain caps the fusion (F) protein of the Paramyxoviridae virus at its apex; or
administering to the target cell an antibody, wherein the antibody comprises an antigen binding site comprising an amino acid sequence encoding a globular head domain of a receptor binding protein of the Paramyxoviridae virus or a fragment thereof, wherein the globular head domain caps the fusion (F) protein of the Paramyxoviridae virus at its apex.

13. The method of claim 12, wherein the fragment of the receptor binding protein comprises a loop and/or a beta sheet.

14. A method of stabilizing a fusion (F) protein of a Paramyxoviridae virus in a pre-fusion configuration, the method comprising administering to the F protein a therapeutic peptide or protein comprising an amino acid sequence of 5-30 residues in length encoding a globular head domain of a receptor binding protein of the Paramyxoviridae virus or a fragment thereof, wherein:
the globular head domain caps the F protein of the Paramyxoviridae virus at its apex; and
the fragment of the receptor binding protein comprises a loop and/or a beta sheet.
